(19) United States
(12) Patent Application Publication
(10) Pub. No.: US 2026/0083459 A1
(43) Pub. Date: Mar. 26, 2026

(54) PATIENT-SPECIFIC SURGICAL GUIDES, SYSTEMS, AND METHODS OF USE

(71) Applicant: Paragon 28, Inc., Englewood, CO (US)

(72) Inventors: Adam JENSEN, Golden, CO (US); Laura Zagrocki BRINKER, Lone Tree, CO (US); Albert DACOSTA, Lone Tree, CO (US); Jakob WOLYNSKI, Arvada, CO (US)

(73) Assignee: Paragon 28, Inc., Englewood, CO (US)

(21) Appl. No.: 19/407,574

(22) Filed: Dec. 3, 2025

Related U.S. Application Data
(63) Continuation of application No. PCT/US2024/032604, filed on Jun. 5, 2024.
(60) Provisional application No. 63/506,249, filed on Jun. 5, 2023.
(60) Provisional application No. 63/615,509, filed on Dec. 28, 2023.

(51) Int. Cl. A61B 17/15

(52) U.S. Cl. CPC A61B 17/15

(57) ABSTRACT

A surgical system includes a first guide component that is couplable with a first metatarsal, and a second guide component configured to releasably couple with a medial cuneiform. The surgical system also includes a cut guide and a correction guide. A surgical method is disclosed that includes the steps of coupling a first guide with a first metatarsal of a patient using at least one first stabilization wire; removing the first guide from the first metatarsal; coupling a second guide with the medial cuneiform using at least one second stabilization wire; removing the second guide from the medial cuneiform; engaging a cut guide with the first metatarsal via the at least one first stabilization wire; disengaging the cut guide from the first metatarsal; engaging the cut guide with the medial cuneiform via the at least one second stabilization wire; and disengaging the cut guide from the medial cuneiform.

### CROSS RELATED APPLICATIONS

This application is a bypass continuation of International Patent Application PCT/US2024/032604, filed Jun. 5, 2024, and entitled “Patient-Specific Surgical Guides, Systems, and Methods of Use,” which claims benefit of U.S. Provisional Application No. 63/506,249 filed on Jun. 5, 2023, and entitled “Patient-Specific Surgical Guides, Systems, and Methods of Use,” and U.S. Provisional Application No. 63/615,509 filed on Dec. 28, 2023, and entitled “Patient-Specific Surgical Guides, Systems, and Methods of Use,” the disclosures of which are hereby incorporated herein by reference in their entirety.

### TECHNICAL FIELD

The present disclosure relates to surgical instruments, guides, and methods of use to be implemented in surgical procedures. The present disclosure relates to podiatric and orthopedic surgical instruments, guides, and methodology to be implemented in various procedures of the foot and/or ankle, for example arthrodesis. More specifically, but not exclusively, the present disclosure relates to surgical instruments, patient-specific guides to be implemented in conjunction with instruments (as well as other components, for example implants, devices, systems, assemblies, etc.) and methods of use for performing arthrodesis procedures of the Lapidus joint.

### BACKGROUND OF THE INVENTION

Many currently available surgical instruments and guides, as well as methodology, do not completely address the needs of patients. Additionally, many currently available surgical instruments, guides, and methodology fail to account for properties of joint anatomy and accordingly can decrease favorability of the outcome for the patient.

### SUMMARY OF THE INVENTION

The present disclosure is directed toward surgical guides for implementation in conjunction with implants, instruments, and methods directed to arthroplasty procedures.

A first aspect of the present disclosure is a surgical system. The surgical system includes a first guide component configured to releasably couple with a first metatarsal of a patient, and a second guide component configured to releasably couple with a medial cuneiform of the patient. The surgical system also includes a cut guide and a correction guide.

According to the first aspect of the present disclosure, the first guide component includes one or more features specific to the anatomy of the patient.

According to the first aspect of the present disclosure, the first guide component includes at least one bone-interfacing surface.

According to the first aspect of the present disclosure, the at least one bone-interfacing surface includes at least one curvature that is complementary to at least one curvature of the first metatarsal of the patient.

According to the first aspect of the present disclosure, the first guide component includes a wire guide, wherein the wire guide includes a pair of openings extending therethrough from a top surface to a bottom surface and establishing fluid communication therebetween.

According to the first aspect of the present disclosure, the wire guide is integral with the first guide component.

According to the first aspect of the present disclosure, the first guide component includes a top surface positioned opposite a body from a bottom surface, and a medial portion opposite the body from a lateral portion, wherein the top and bottom surfaces extend at least partially from the medial side to the lateral side of the body.

According to the first aspect of the present disclosure, at least one of the medial portion and the lateral portion includes an engagement feature extending upward from the top surface.

According to the first aspect of the present disclosure, the first guide component includes one or more features specific to the anatomy of the patient.

According to the first aspect of the present disclosure, the second guide component includes at least one bone-interfacing surface.

According to the first aspect of the present disclosure, the at least one bone-interfacing surface includes at least one curvature that is complementary to at least one curvature of the medial cuneiform of the patient.

According to the first aspect of the present disclosure, the second guide component includes a wire guide, wherein the wire guide includes a pair of openings extending therethrough from a top surface to a bottom surface and establishing fluid communication therebetween.

According to the first aspect of the present disclosure, the wire guide is integral with the first guide component.

According to the first aspect of the present disclosure, the second guide component includes a top surface positioned opposite a body from a bottom surface, and a medial portion opposite the body from a lateral portion, wherein the top and bottom surfaces extend at least partially from the medial side to the lateral side of the body.

According to the first aspect of the present disclosure, at least one of the medial portion and the lateral portion includes an engagement feature extending upward from the top surface.

According to the first aspect of the present disclosure, the system includes at least one first stabilization wire configured to couple the second guide component with the first metatarsal of the patient and at least one second stabilization wire configured to releasably couple the second guide component with the medial cuneiform of the patient.

According to the first aspect of the present disclosure, the cut guide is configured to engage with the first metatarsal of the patient via the at least one first stabilization wire.

According to the first aspect of the present disclosure, the cut guide is configured to engage with the medial cuneiform of the patient via the at least one second stabilization wire.

A second aspect of the present disclosure is directed to a surgical method. The surgical method includes coupling a first guide with a first metatarsal of a patient using at least one first stabilization wire, removing the first guide from the first metatarsal, coupling a second guide with a medial cuneiform of a patient using at least one second stabilization wire, and removing the second guide from the medial cuneiform. The surgical method also includes engaging a cut guide with the first metatarsal of the patient via the at least one first stabilization wire, disengaging the cut guide from the first metatarsal of the patient, engaging the cut guide with the medial cuneiform of the patient via the at least one second stabilization wire, and disengaging the cut guide from the medial cuneiform of the patient.

According to the second aspect of the present disclosure, the surgical method includes performing at least one resection cut to the first metatarsal, wherein the at least one resection cut is guided by a slot disposed in the cut guide, and performing at least one resection cut to the medial cuneiform, wherein the at least one resection cut is guided by the slot positioned in the cut guide.

A third aspect of the present disclosure is directed to a surgical system. The surgical system includes a first guide, a second guide, and a correction guide.

According to the third aspect of the present disclosure, the first guide is configured to guide a cut to a proximal end of a first metatarsal of a patient.

According to the third aspect of the present disclosure, the first guide is a patient-specific guide.

According to the third aspect of the present disclosure, the second guide is configured to guide a cut to a distal end of a medial cuneiform of a patient.

According to the third aspect of the present disclosure, the second guide is a patient-specific guide.

According to the third aspect of the present disclosure, the correction guide is configured to releasably couple with the first metatarsal and medial cuneiform of the patient.

According to the third aspect of the present disclosure, the correction guide is modular and comprises a proximal portion and a distal portion that are releasably couplable with one another.

According to the third aspect of the present disclosure, the first guide includes a cut slot positioned at a proximal portion thereof, and at least one opening configured to receive a k-wire at least partially therethrough so as to facilitate coupling of the first guide with the first metatarsal.

According to the third aspect of the present disclosure, the first guide further comprises at least one engagement feature.

According to the third aspect of the present disclosure, the at least one opening extends at least partially through the at least one engagement feature.

According to the third aspect of the present disclosure, the at least one engagement feature includes a medial engagement feature and a lateral engagement feature, and the at least one opening includes a first opening extending at least partially through the medial engagement feature and a second opening extending at least partially through the lateral engagement feature.

According to the third aspect of the present disclosure, the second guide includes a cut slot positioned at a distal portion thereof, and at least one opening configured to receive a k-wire at least partially therethrough so as to facilitate coupling of the first guide with the medial cuneiform.

According to the third aspect of the present disclosure, the second guide further comprises at least one engagement feature.

According to the third aspect of the present disclosure, the at least one opening extends at least partially through the at least one engagement feature.

A fourth aspect of the present disclosure is directed to a surgical system. The system includes a first guide, which includes a cut slot positioned as a proximal portion thereof, a first engagement feature, a second engagement feature, a first opening extending at least partially through the first engagement feature, and a second opening extending at least partially through the second engagement feature. The system also includes a second guide which includes a cut slot positioned as a distal portion thereof, a first engagement feature, a second engagement feature, a first opening extending at least partially through the first engagement feature, and a second opening extending at least partially through the second engagement feature. The system also includes a correction guide, which includes a proximal portion and a distal portion, with the proximal and distal portions releasably couplable with one another.

According to the fourth aspect of the present disclosure, the first and second guides are patient-specific guides.

According to the fourth aspect of the present disclosure, the correction guide is patient-specific.

According to the fourth aspect of the present disclosure, the first guide is configured to be releasably couplable with a first metatarsal of a patient, and wherein the second guide is configured to be releasably couplable with a medial cuneiform of a patient.

A fifth aspect of the present disclosure is directed to a method of correcting a bunion. The method includes engaging a first guide with a first metatarsal of a patient, releasably coupling the first guide with the first metatarsal of the patient using one or more stabilization wires, and performing a resection cut to the proximal portion of the first metatarsal through a cut slot positioned at a proximal end of the first guide. The method also includes engaging a second guide with a medial cuneiform of the patient, releasably coupling the second guide with the medial cuneiform of the patient using one or more stabilization wires, and performing a resection cut to the distal portion of the medial cuneiform through a cut slot positioned at a distal end of the second guide. The method also includes engaging a correction guide with the first metatarsal and medial cuneiform to bias the first metatarsal and medial cuneiform into a corrected position.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top, front perspective view of an exemplary system for use in performing a surgical procedure, in accordance with the present disclosure;

FIG. 2 is a rear perspective of the exemplary system of FIG. 1 for use in performing a surgical procedure, in accordance with the present disclosure;

FIG. 3 is side view of components of the exemplary system of FIG. 1 for use in performing a surgical procedure, in accordance with the present disclosure;

FIG. 4 is a top view of an exemplary portion of the exemplary system of FIG. 1 for use in performing a surgical procedure, in accordance with the present disclosure;

FIG. 5 is an alternate top view of an exemplary portion of the exemplary system of FIG. 1 for use in performing a surgical procedure, in accordance with the present disclosure;

FIG. 6 is a top perspective view of an exemplary component of the exemplary system of FIG. 1 for use in performing a surgical procedure, in accordance with the present disclosure;

FIG. 7 is a bottom perspective view of the exemplary component of FIG. 6 of the exemplary system of FIG. 1 for use in performing a surgical procedure, in accordance with the present disclosure;

FIG. 8 is a top perspective view of an exemplary component of the exemplary system of FIG. 1 for use in performing a surgical procedure, in accordance with the present disclosure;

FIG. 9 is a bottom perspective view of the exemplary component of FIG. 8 of the exemplary system of FIG. 1 for use in performing a surgical procedure, in accordance with the present disclosure;

FIG. 10 is a top, front perspective view of an exemplary portion of an exemplary system for use in performing a surgical procedure, in accordance with the present disclosure;

FIG. 11 is a top view of an exemplary component of the exemplary system of FIG. 10 for use in performing a surgical procedure, in accordance with the present disclosure;

FIG. 12 is a rear view of the exemplary component of FIG. 11 of the exemplary system of FIG. 10 for use in performing a surgical procedure, in accordance with the present disclosure;

FIG. 13 is a side view of the exemplary component of FIG. 11 of the exemplary system of FIG. 10 for use in performing a surgical procedure, in accordance with the present disclosure;

FIG. 14 is a top, front perspective view of an exemplary portion of the exemplary system of FIG. 10 for use in performing a surgical procedure, in accordance with the present disclosure;

FIG. 15 is a top view of an exemplary component of FIG. 14 of the exemplary system of FIG. 10 for use in performing a surgical procedure, in accordance with the present disclosure;

FIG. 16 is a side perspective view of the exemplary component of FIG. 14 of the exemplary system of FIG. 10 for use in performing a surgical procedure, in accordance with the present disclosure;

FIG. 17 is a side view of the exemplary component of FIG. 14 of the exemplary system of FIG. 10 for use in performing a surgical procedure, in accordance with the present disclosure;

FIG. 18 is a top, front perspective view of an exemplary portion including exemplary components of an exemplary system for use in performing a surgical procedure, in accordance with the present disclosure;

FIG. 19 is a top view of the exemplary components of the exemplary portion of FIG. 18 of an exemplary system for use in performing a surgical procedure, in accordance with the present disclosure;

FIG. 20 is a side view of the exemplary portion including exemplary components of FIG. 18 of an exemplary system for use in performing a surgical procedure, in accordance with the present disclosure;

FIG. 21 is a rear perspective view of the exemplary components of the exemplary portion of FIG. 18 of an exemplary system for use in performing a surgical procedure, in accordance with the present disclosure;

FIG. 22 is a side perspective view of an exemplary component of the exemplary portion of an exemplary system for use in performing a surgical procedure, in accordance with the present disclosure;

FIG. 23 is a bottom perspective view of the exemplary component of FIG. 22 of the exemplary portion of FIG. 18 of an exemplary system for use in performing a surgical procedure, in accordance with the present disclosure;

FIG. 24 is a side view of the exemplary component of FIG. 22 of the exemplary portion of FIG. 18 of an exemplary system for use in performing a surgical procedure, in accordance with the present disclosure;

FIG. 25 is a top, front perspective view of an exemplary portion including exemplary components of the exemplary system of FIG. 18 for use in performing a surgical procedure, in accordance with the present disclosure;

FIG. 26 is a top perspective view of the exemplary portion of FIG. 25 of an exemplary system for use in performing a surgical procedure, in accordance with the present disclosure;

FIG. 27 is a side perspective view of the exemplary portion of FIG. 25 of an exemplary system for use in performing a surgical procedure, in accordance with the present disclosure;

FIG. 28 is a top perspective view of an exemplary component of the exemplary portion of FIG. 25 of an exemplary system for use in performing a surgical procedure, in accordance with the present disclosure;

FIG. 29 is a bottom perspective view of the exemplary component of FIG. 28 of the exemplary portion of FIG. 25 of an exemplary system for use in performing a surgical procedure, in accordance with the present disclosure;

FIG. 30 is a side view of the exemplary component of FIG. 28 of the exemplary portion of FIG. 25 of an exemplary system for use in performing a surgical procedure, in accordance with the present disclosure;

FIG. 31 is a top perspective view of components of the exemplary portions of FIGS. 18 and 25 of an exemplary system for use in performing a surgical procedure, in accordance with the present disclosure;

FIG. 32 is a top view of components of an exemplary system for implementation in performing a surgical procedure, in accordance with the present disclosure;

FIG. 33 is an alternate top view of the components of FIG. 32 of an exemplary system for use in performing a surgical procedure, in accordance with the present disclosure;

FIG. 34 is a top view of a component of FIG. 32 of an exemplary system for use in performing a surgical procedure, in accordance with the present disclosure;

FIG. 35 is a side view of the component of FIG. 32 of an exemplary system for use in performing a surgical procedure, in accordance with the present disclosure;

FIG. 36 is an alternate side view of the component of FIG. 32 of an exemplary system for use in performing a surgical procedure, in accordance with the present disclosure;

FIG. 37 is a front view of the component of FIG. 32 of an exemplary system for use in performing a surgical procedure, in accordance with the present disclosure;

FIG. 38 is a rear view of the component of FIG. 32 of an exemplary system for use in performing a surgical procedure, in accordance with the present disclosure;

FIG. 39 is a bottom view of the component of FIG. 32 of an exemplary system for use in performing a surgical procedure, in accordance with the present disclosure;

FIG. 40 is a top view of the components of an exemplary system for use in performing a surgical procedure, in accordance with the present disclosure;

FIG. 41 is an alternate top view of the components of an exemplary system for use in performing a surgical procedure, in accordance with the present disclosure;

FIG. 42 is a top perspective view of the components of an exemplary system for use in performing a surgical procedure, in accordance with the present disclosure;

FIG. 43 is a front, elevated, perspective view of the components of an exemplary system for use in performing a surgical procedure, in accordance with the present disclosure;

FIG. 44 is a side perspective view of the components of FIG. 43 of an exemplary system for use in performing a surgical procedure, in accordance with the present disclosure;

FIG. 45 is a rear, elevated, perspective view of the components of FIG. 43 of an exemplary system for use in performing a surgical procedure, in accordance with the present disclosure;

FIG. 46 is a top view of the components of FIG. 43 of an exemplary system for use in performing a surgical procedure, in accordance with the present disclosure;

FIG. 47 is an alternate rear, elevated, perspective view of the components of FIG. 43 of an exemplary system for use in performing a surgical procedure, in accordance with the present disclosure;

FIG. 48 is a rear view of the components of FIG. 43 of an exemplary system for use in performing a surgical procedure, in accordance with the present disclosure;

FIG. 49 is a front, elevated, perspective view of the components of FIG. 43 of an exemplary system for use in performing a surgical procedure, in accordance with the present disclosure;

FIG. 50 is a rear, elevated, perspective view of the components of FIG. 43 of an exemplary system for use in performing a surgical procedure, in accordance with the present disclosure;

FIG. 51 is a top view of the components of FIG. 43 of an exemplary system for use in performing a surgical procedure, in accordance with the present disclosure;

FIG. 52 is a bottom view of the components of FIG. 43 of an exemplary system for use in performing a surgical procedure, in accordance with the present disclosure;

FIG. 53 is a front view of the components of FIG. 43 of an exemplary system for use in performing a surgical procedure, in accordance with the present disclosure;

FIG. 54 is a rear view of the components of FIG. 43 of an exemplary system for use in performing a surgical procedure, in accordance with the present disclosure;

FIG. 55 is a side view of the components of FIG. 43 of an exemplary system for use in performing a surgical procedure, in accordance with the present disclosure;

FIG. 56 is an alternate side view of the components of FIG. 43 of an exemplary system for use in performing a surgical procedure, in accordance with the present disclosure;

FIG. 57 is a front, elevated, perspective view of an exemplary component of an exemplary system for use in performing a surgical procedure, in accordance with the present disclosure;

FIG. 58 is a rear, elevated, perspective view of the component of FIG. 57 of an exemplary system for use in performing a surgical procedure, in accordance with the present disclosure;

FIG. 59 is a front view of the component of FIG. 57 of an exemplary system for use in performing a surgical procedure, in accordance with the present disclosure;

FIG. 60 is a side view of the component of FIG. 57 of an exemplary system for use in performing a surgical procedure, in accordance with the present disclosure;

FIG. 61 is a rear view of the component of FIG. 57 of an exemplary system for use in performing a surgical procedure, in accordance with the present disclosure;

FIG. 62 is a front, elevated, perspective view of the exemplary component of FIG. 57 of an exemplary system for use in performing a surgical procedure, in accordance with the present disclosure;

FIG. 63 is a rear, elevated, perspective view of the component of FIG. 57 of an exemplary system for use in performing a surgical procedure, in accordance with the present disclosure;

FIG. 64 is a top view of the exemplary component of FIG. 57 of an exemplary system for use in performing a surgical procedure, in accordance with the present disclosure;

FIG. 65 is a bottom view of the component of FIG. 57 of an exemplary system for use in performing a surgical procedure, in accordance with the present disclosure;

FIG. 66 is a front view of the component of FIG. 57 of an exemplary system for use in performing a surgical procedure, in accordance with the present disclosure;

FIG. 67 is a rear view of the component of FIG. 57 of an exemplary system for use in performing a surgical procedure, in accordance with the present disclosure;

FIG. 68 is a side view of the component of FIG. 57 of an exemplary system for use in performing a surgical procedure, in accordance with the present disclosure;

FIG. 69 is an alternate side view of the component of FIG. 57 of an exemplary system for use in performing a surgical procedure, in accordance with the present disclosure;

FIG. 70 is a front, elevated, perspective view of the components of an exemplary system for use in performing a surgical procedure, in accordance with the present disclosure;

FIG. 71 is a rear, elevated, perspective view of the components of FIG. 70 of an exemplary system for use in performing a surgical procedure, in accordance with the present disclosure;

FIG. 72 is an alternate perspective view of the components of FIG. 70 of an exemplary system for use in performing a surgical procedure, in accordance with the present disclosure;

FIG. 73 is a top view of the components of FIG. 70 of an exemplary system for use in performing a surgical procedure, in accordance with the present disclosure;

FIG. 74 is an exploded, perspective view of the components of FIG. 70 of an exemplary system for use in performing a surgical procedure, in accordance with the present disclosure;

FIG. 75 is a front, elevated, perspective view of the components of FIG. 70 of an exemplary system for use in performing a surgical procedure, in accordance with the present disclosure;

FIG. 76 is a rear, elevated, perspective view of the components of FIG. 70 of an exemplary system for use in performing a surgical procedure, in accordance with the present disclosure;

FIG. 77 is a top view of the components of FIG. 70 of an exemplary system for use in performing a surgical procedure, in accordance with the present disclosure;

FIG. 78 is a bottom view of the components of FIG. 70 of an exemplary system for use in performing a surgical procedure, in accordance with the present disclosure;

FIG. 79 is a front view of the components of FIG. 70 of an exemplary system for use in performing a surgical procedure, in accordance with the present disclosure;

FIG. 80 is a rear view of the components of FIG. 70 of an exemplary system for use in performing a surgical procedure, in accordance with the present disclosure;

FIG. 81 is a side view of the components of FIG. 70 of an exemplary system for use in performing a surgical procedure, in accordance with the present disclosure;

FIG. 82 is an alternate side view of the components of FIG. 70 of an exemplary system for use in performing a surgical procedure, in accordance with the present disclosure;

FIG. 83 is a perspective view of a step of a surgical procedure using an exemplary system for use in performing a surgical procedure, in accordance with the present disclosure;

FIG. 84 is a perspective view of a step of a surgical procedure using an exemplary system for use in performing a surgical procedure, in accordance with the present disclosure;

FIG. 85 is a perspective view of a step of a surgical procedure using an exemplary system for use in performing a surgical procedure, in accordance with the present disclosure;

FIG. 86 is a perspective view of a step of a surgical procedure using an exemplary system for use in performing a surgical procedure, in accordance with the present disclosure;

FIG. 87 is a perspective view of a step of a surgical procedure using an exemplary system for use in performing a surgical procedure, in accordance with the present disclosure;

FIG. 88 is a perspective view of a step of a surgical procedure using an exemplary system for use in performing a surgical procedure, in accordance with the present disclosure;

FIG. 89 is a perspective view of a step of a surgical procedure using an exemplary system for use in performing a surgical procedure, in accordance with the present disclosure;

FIG. 90 is a perspective view of a step of a surgical procedure using an exemplary system for use in performing a surgical procedure, in accordance with the present disclosure;

FIG. 91 is a perspective view of a step of a surgical procedure using an exemplary system for use in performing a surgical procedure, in accordance with the present disclosure;

FIG. 92 is a perspective view of a step of a surgical procedure using an exemplary system for use in performing a surgical procedure, in accordance with the present disclosure;

FIG. 93 is a perspective view of a step of a surgical procedure using an exemplary system for use in performing a surgical procedure, in accordance with the present disclosure;

FIG. 94 is a perspective view of a step of a surgical procedure using an exemplary system for use in performing a surgical procedure, in accordance with the present disclosure;

FIG. 95 is a perspective view of a step of a surgical procedure using an exemplary system for use in performing a surgical procedure, in accordance with the present disclosure;

FIG. 96 is a perspective view of a step of a surgical procedure using an exemplary system for use in performing a surgical procedure, in accordance with the present disclosure;

FIG. 97 is a perspective view of a step of a surgical procedure using an exemplary system for use in performing a surgical procedure, in accordance with the present disclosure;

FIG. 98 is a perspective view of a step of a surgical procedure using an exemplary system for use in performing a surgical procedure, in accordance with the present disclosure;

FIG. 99 is a perspective view of a step of a surgical procedure using an exemplary system for use in performing a surgical procedure, in accordance with the present disclosure;

FIG. 100 is a perspective view of a step of a surgical procedure using an exemplary system for use in performing a surgical procedure, in accordance with the present disclosure;

FIG. 101 is a perspective view of a step of a surgical procedure using an exemplary system for use in performing a surgical procedure, in accordance with the present disclosure;

FIG. 102 is a perspective view of a step of a surgical procedure using an exemplary system for use in performing a surgical procedure, in accordance with the present disclosure;

FIG. 103 is a perspective view of a step of a surgical procedure using an exemplary system for use in performing a surgical procedure, in accordance with the present disclosure;

FIG. 104 is a perspective view of a step of a surgical procedure using an exemplary system for use in performing a surgical procedure, in accordance with the present disclosure;

FIG. 105 is a perspective view of a step of a surgical procedure using an exemplary system for use in performing a surgical procedure, in accordance with the present disclosure;

FIG. 106 is a perspective view of a step of a surgical procedure using an exemplary system for use in performing a surgical procedure, in accordance with the present disclosure;

FIG. 107 is a perspective view of a step of a surgical procedure using an exemplary system for use in performing a surgical procedure, in accordance with the present disclosure; and

FIG. 108 is a perspective view of a step of a surgical procedure using an exemplary system for use in performing a surgical procedure, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in International PCT Application No. PCT/US2018/20046, filed on Feb. 27, 2018, and entitled Intramedullary Nail Alignment Guides, Fixation Guides, Devices, Systems, and Methods of Use; International PCT Application No. PCT/US2018/64368, filed on Dec. 17, 2018, and entitled Alignment Guides, Cut Guides, Systems and Methods of Use and Assembly; International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or U.S. Pat. No. D9042010 filed on Apr. 9, 2019 and entitled Cut Guide; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in United Stated Provisional Application No. 63/173,043, filed Apr. 9, 2021 and entitled Surgical Instruments, Guides, and Methods of Use; and/or International PCT Application No. PCT/US2018/20046, filed on Feb. 27, 2018, and entitled Intramedullary Nail Alignment Guides, Fixation Guides, Devices, Systems, and Methods of Use; and/or International PCT Application No. PCT/US2018/64368, filed on Dec. 17, 2018, and entitled Alignment Guides, Cut Guides, Systems and Methods of Use and Assembly; and/or International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or U.S. Pat. No. D904,2010 filed on Apr. 9, 2019 and entitled Cut Guide; and/or U.S. Provisional Patent Application No. 63/262,845 filed on Oct. 21, 2021 and entitled Surgical Instruments, Guides, and/Methods of Use; and/or U.S. Provisional Patent Application No. 63/304,144 filed on Jan. 28, 2022 and entitled Surgical Instruments, Guides, and Methods of Use; and/or U.S. Provisional Patent Application No. 63/497,509 filed on Apr. 21, 2023 and entitled Three-Dimensional Model Generation and Surgical Planning Based Thereon, which are all hereby incorporated herein by reference in their entireties.

Procedures to address deformities such as bunions and anatomical structures of and around the Lapidus joint frequently require the positioning/repositioning and/or rotation/derotation of the first metatarsal. Referred to herein as the “Lapidus” joint, this joint may also be known and referred to as the metatarsocuneiform joint. It is common for a procedure of the Lapidus joint (e.g., fusion/arthrodesis) to require that the first metatarsal be manipulated by applying one or more forces to the first metatarsal. In some procedures, this manipulation is necessary before any cutting and/or preparation and subsequent fusion of the Lapidus joint can take place. In evaluating a Lapidus joint deformity, two different criteria are typically analyzed for correction. One of these criteria is the intramedullary angle formed between the longitudinal axes of the first metatarsal and the second metatarsal. Bunion deformities and other conditions of the Lapidus joint often include the first metatarsal shifting medially from a normal anatomical position, thus increasing the IM angle between the first and second metatarsals from what can be considered an anatomically correct range of angle measures. Rotation of the first metatarsal is also analyzed, as bunion deformities and other conditions of the Lapidus joint commonly include a first metatarsal that has rotated substantially in the frontal plane in a substantially clockwise direction (when viewed from an anterior to posterior direction). Commonly, a Lapidus joint procedure such as those mentioned previously requires manipulation of the first metatarsal so as to a) correct (e.g., decrease) the IM angle between the first and second metatarsals by applying a substantially lateral force to the first metatarsal; and/or b) derotate the first metatarsal which as rotated from a normal anatomical position by applying a rotational force in a substantially counterclockwise direction when the first metatarsal is viewed in an anterior to posterior direction.

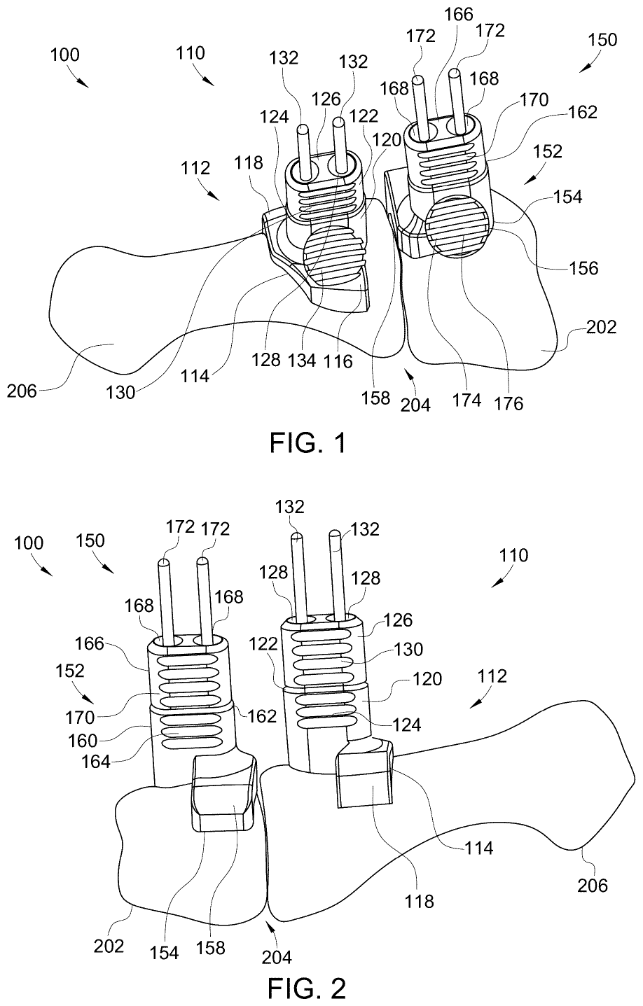

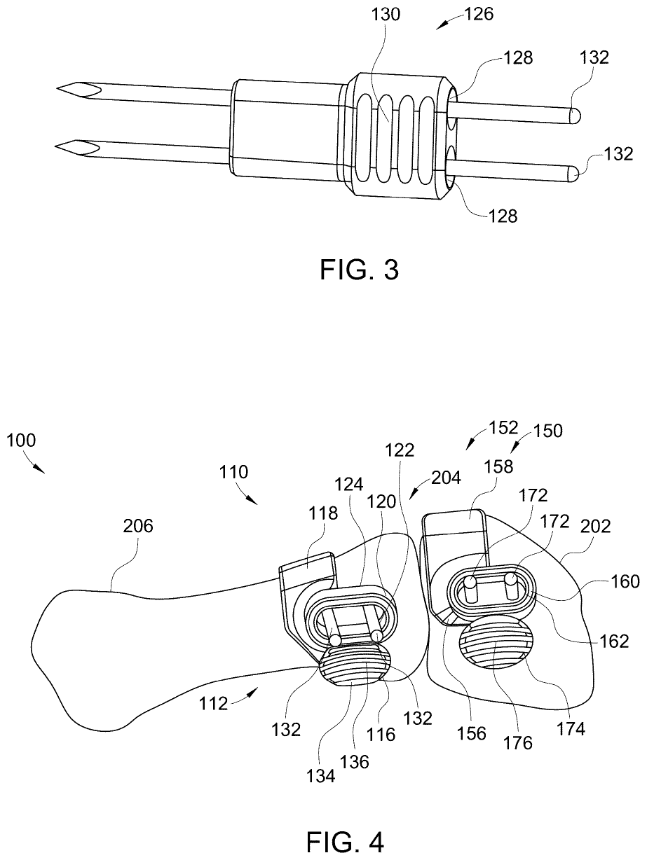

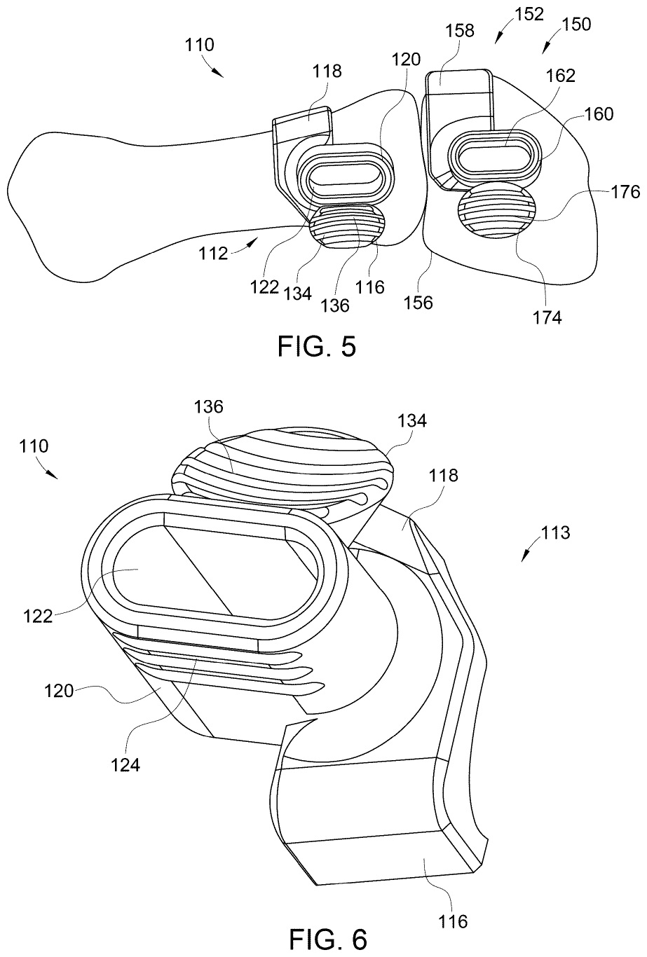

Referring to the drawings, wherein like reference numerals are used to indicate like or analogous components throughout the several views, and with particular reference to FIGS. 1-9, there is illustrated an exemplary embodiment of a system 100 for performing at least a portion of a Lapidus joint procedure. In some aspects, the system 100 may be implemented in conjunction with other components common to orthopedic procedures, for example drills, reciprocating/sagittal saws, and other powered instruments which, for the sake of brevity, are not shown herein. The system 100 as shown is a patient-specific system and, accordingly, one or more components or features thereof may correspond to a specific portion of a specific anatomy of a specific patient. For example, a component in the system 100 may include a surface that is complementary in concavity to a convexity of a portion of a bone of a patient with which the portion of the component may interface. In some aspects, one or more components of the system 100 may be generated based on analysis of an input that includes patient data, for example patient imaging data including, for example, a CT or x-ray data.

The system 100 includes a metatarsal guide 110 configured to interface and releasably couple with a first metatarsal 206 of a patient, as well as a cuneiform guide 150 configured to interface and releasably couple with a medial cuneiform 202 of a patient as shown in at least FIGS. 1-2. As shown, the first metatarsal guide 110 is configured to interface with a proximal portion (e.g., the base) of the first metatarsal 206, whereas the cuneiform guide 150 is configured to interface with a distal portion of the medial cuneiform 202. In some aspects, one or both of the guides 110, 150 may include one or more features which, when the guide is placed in a desired position on the appropriate anatomy, extend into or near a joint space 204 of the metatarsocuneiform joint (e.g., the Lapidus joint). Each of the guides 110, 150 are shown to be releasably couplable with the first metatarsal 206 and the medial cuneiform 202, respectively. In some aspects, the guides 110, 150 may include features to facilitate releasable coupling therebetween, for example a dovetail or other similar feature.

The metatarsal guide 110 is shown to include a body 112, wherein the body includes a medial portion 116 substantially opposite the guide 110 from a lateral portion 118. As shown, the body 112 includes a lower surface 114 (at least a portion of which is configured to interface with a surface of the first metatarsal 206) extending from the medial portion 116 to the lateral portion 118. The lower surface 114 may be a partial surface, for example including one or more concavities and/or recesses therein. In some aspects, the lower surface 114 may include one or more curvatures, concavities, convexities, or changes in elevation configured to accommodate a surface of the first metatarsal 206 and/or various features of the metatarsal guide 110 configured to facilitate placement and coupling of the metatarsal guide 110 with the first metatarsal 206.

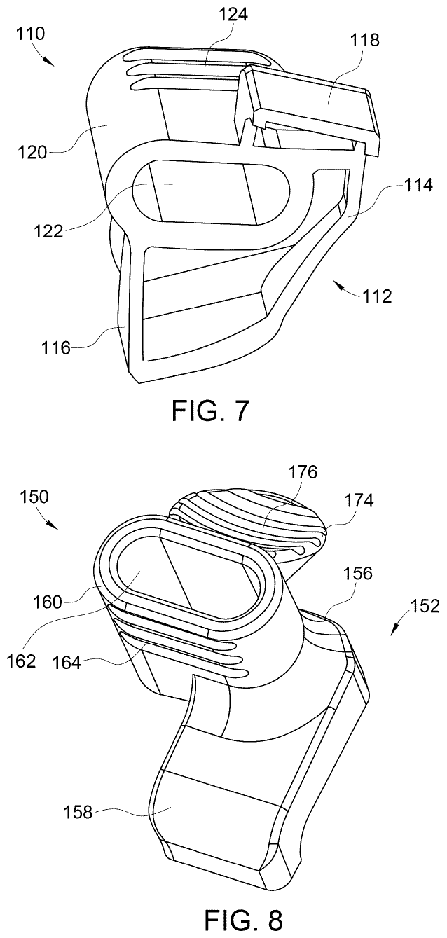

In another example, as shown in FIGS. 6-7, the medial portion 116 includes a curvature which includes a radiused or otherwise arcuate portion extending between a first plane of the lower surface 114 that is substantially parallel with a portion of the surface of the first metatarsal 206 and a second plane of the lower surface 114 that is substantially perpendicular relative to the first plane. In the previous example, the radiused or arcuate portion of the lower surface 114 may be contoured to a curvature of the first metatarsal 206 of the patient. As shown, the lateral portion 118 includes a similarly curved geometry in order provide structural support for the contouring of the metatarsal guide 110. This feature may generally take the form of a “hook” shape or other similar geometry and extend around the top portion of the first metatarsal 206 to contact a “flat” anatomical feature found on the lateral portion of the anatomy of the first metatarsal 206. The medial portion 116 may include a similar feature to that of the lateral portion 118 which, in conjunction with the lower surface 114, provides structural support for a contouring feature of the lower surface 114 that is configured to contact one or more portions of the medial anatomy of the first metatarsal. The structural features of the lower surface 114 in conjunction with the medial and/or lateral portions 116, 118 of the metatarsal guide 110 may be configured to facilitate placement of the metatarsal guide in a desired position by referencing known anatomical features of the topology of the first metatarsal 206.

As shown in FIGS. 1-9, the medial portion 116 of the metatarsal guide 110 includes an engagement feature 134 which, as shown, includes for example, a substantially circular feature extending from and positioned above (e.g., superior relative to) the medial portion 116. The engagement feature is shown to include a texture 136, shown in an exemplary embodiment, for example, as a plurality of ridges, which may be gripped or otherwise held by a physician when placing the metatarsal guide 110 in a desired position on the first metatarsal 206 (or when performing other operations involving the metatarsal guide 110). In some aspects, the engagement feature 134 may have an alternate position about the metatarsal guide 110, for example, extending from and/or above the lateral portion 118. Further, the metatarsal guide 110 may include multiple engagement features 134, for example, extending from or above both the medial and lateral portions 116, 118.

As shown in FIGS. 1-9, a shaft 120 is shown to extend upward from a central portion of the body 112 disposed substantially between the medial and lateral portions 116, 118. As shown, the shaft 120 includes a substantially oval or elliptically shaped cross-sectional geometry and is centered about a central axis extending substantially perpendicular to at least a portion of the surface of the first metatarsal 206. A texture 124 is disposed on at least a portion of the outer surface of the shaft 120, shown in an exemplary embodiment, for example, as a plurality of ridges, so as to facilitate gripping of the shaft 120 by a physician or other medical personnel. The shaft 120 is shown to define a cavity 122 therein, where the cavity 122 establishes fluid communication between an upper portion of the shaft 120 and a lower portion of the shaft 120 (which abuts the lower surface 114) configured to contact or be positioned adjacent the surface of the first metatarsal 206.

The system 100 further includes a wire guide 126 configured to facilitate the releasable coupling of the metatarsal guide 110 with the first metatarsal 206. The wire guide 126 is shown to include a substantially oval or elliptical prism-shaped geometry (and accordingly a similarly shaped cross-sectional geometry). At least a portion of the wire guide 126 (as shown, the lower portion) is configured to have a complementary geometry to that of the cavity 122 (with a lesser lateral dimension) such that the portion of the wire guide 126 may be received by the cavity 122 therein. Further, at least a portion of the wire guide 126 is shown to have a greater lateral dimension than that of the cavity 122 (which may function as a depth stop feature) so as to limit the depth of insertion of the wire guide 126 within the cavity 122. The wire guide 126 is further shown to include at least one bore (which, as shown in at least FIGS. 1-3 includes a pair of bores and will be referred to hereinafter as such) 128 extending from a top surface of the wire guide 126 through a body portion thereof to a bottom surface of the wire guide 126. As shown in at least FIGS. 1-3, the bores 128 are centered about respective central axes and have a substantially cylindrical geometry. Further, the bores 128 are configured such that the central axes thereof are positioned substantially parallel to one another (although in some aspects, the bores 128 and central axes thereof may be angled so as to achieve converging, diverging, or other non-parallel, oblique trajectories). As shown, the bores 128 are each configured to receive one of the stabilization wires 132 therein and therethrough and accordingly may be sized (e.g., diametrically, length-wise, etc.) to accommodate one or more sizes of the stabilization wires 132.

The metatarsal guide 110 may be implemented in conjunction with other components of the system 100 to couple the metatarsal guide 110 with the first metatarsal 206 of the patient. The metatarsal guide 110 may first be positioned and placed by a physician such that at least a portion of the lower surface 114 interfaces with the surface of the first metatarsal 206 of the patient. The metatarsal guide 110 may include one or more features, for example, the bottom surface 114 and/or the medial and/or lateral portions 116, 118, that facilitate placement of the metatarsal guide 110 in a desired position (and may provide haptic feedback to a physician confirming proper placement/positioning of the guide 110). For example, the lower surface 114 and the medial portion 116 may collectively include a curvature configured to interface with a medial portion of the surface of the first metatarsal 206. Further, the lower surface and/or the lateral portion 118 of the metatarsal guide 110 may include an extension or other feature configured to wrap around a portion of the first metatarsal 206 so as to interface with and reference an anatomical feature of the first metatarsal 206.

Once the metatarsal guide 110 has been placed in a desired position, the wire guide 126 may be inserted such that at least a portion of the guide is received by and positioned therein the cavity 122 of the metatarsal guide 110. In placing the wire guide 126 at least partially within the cavity 122, a physician may grip the metatarsal guide 110 via the texture 124 provided thereon, and/or may also grip the wire guide 126 by a texture 130 (shown in FIGS. 1-3 as a plurality of ridges) disposed on an outer surface thereof. Various features of the metatarsal guide 110, for example, those of the lower surface 114 in conjunction with the medial and lateral portions 116, 118 as described previously herein may provide haptic feedback to a physician in that the one or more features retain the metatarsal guide 110 in a desired position when the feature(s) contact the corresponding complementary anatomical features to which they are contoured and reference. In some aspects, a physician may elect to place at least a portion of the wire guide 126 within the cavity 122 prior to positioning the metatarsal guide 110 on the first metatarsal 206 of the patient. Once the metatarsal guide 110 is placed in a desired position and the wire guide 126 has been inserted at least partially within the cavity 122 of the metatarsal guide 110, one of the stabilization wires 132 may be placed at least partially within one of the bores 128 such that the pointed tip of the stabilization wire 132 is positioned adjacent the surface of the first metatarsal 206. A physician may then couple the stabilization wire 132 with the first metatarsal 206 using one or more instruments common to orthopedic surgical procedures, for example, a drill or other powered driver. In coupling the stabilization wire 132 with the first metatarsal 206, the metatarsal guide 110 and the wire guide 126 are also releasably coupled with the first metatarsal 206. Once a first stabilization wire 132 has been placed, a second stabilization wire 132 may also be placed according to the same procedure. Following placement of the pair of stabilization wires 132, the wire guide 126 and/or the metatarsal guide 110 may be decoupled from the first metatarsal 206 with the stabilization wires 132 remaining coupled with the first metatarsal 206.

The cuneiform guide 150 is shown to include a body 152, wherein the body includes a medial portion 156 substantially opposite the guide 150 from a lateral portion 158. As shown, the body 152 includes a lower surface 154 (at least a portion of which is configured to interface with a surface of the medial cuneiform 202) extending from the medial portion 156 to the lateral portion 158. The lower surface 154 may be a partial surface, for example including one or more concavities and/or recesses therein. In some aspects, the lower surface 154 may include one or more curvatures, concavities, convexities, or changes in elevation configured to accommodate a surface of the medial cuneiform 202 and/or various features of the cuneiform guide 150 that facilitate placement and coupling of the cuneiform guide 150 with the medial cuneiform 202.

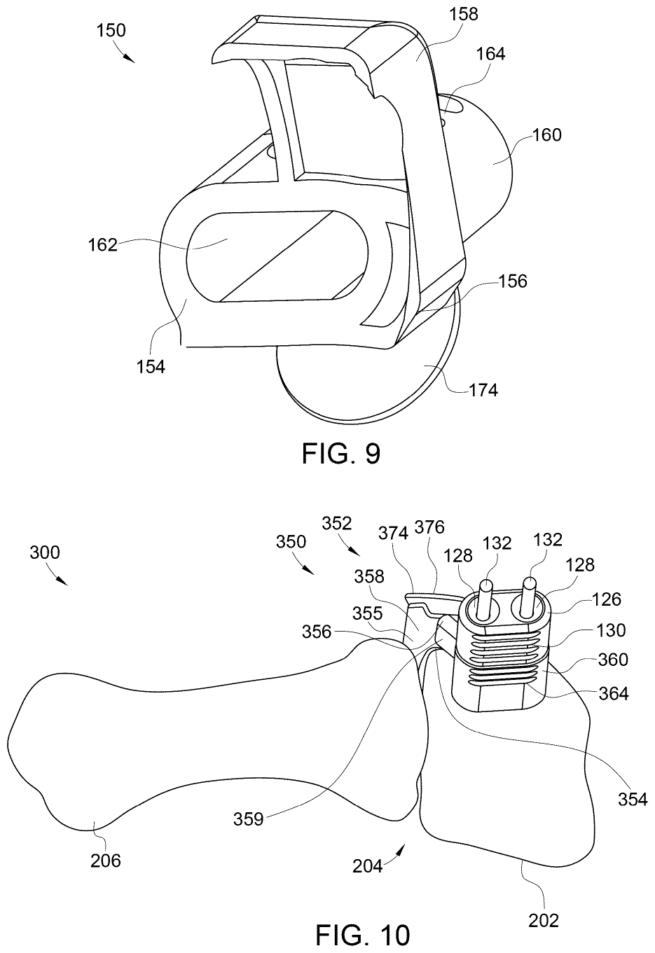

As shown in FIGS. 8-9, the medial portion 156 includes a curvature which includes a radiused or otherwise arcuate portion extending between a first plane of the lower surface 154 that is substantially parallel with a portion of the surface of the medial cuneiform 202 and a second plane of the lower surface 154 that is substantially perpendicular relative to the first plane. In the previous example, the radiused or arcuate portion of the lower surface 154 may be contoured to a curvature of the medial cuneiform 202 of the patient. As shown, the lateral portion 158 includes a similarly curved geometry in order provide structural support for the contouring of the cuneiform guide 150. This feature may generally take the form of a “hook” shape or other similar geometry and extend around the top portion of the medial cuneiform 202 to contact an anatomical feature found on the lateral portion of the anatomy of the medial cuneiform 202. The medial portion 156 may include a similar feature to that of the lateral portion 158 which, in conjunction with the lower surface 154, provides structural support for a contouring feature of the lower surface 154 that is configured to contact one or more portions of the medial anatomy of the first metatarsal 206. The structural features of the lower surface 154 in conjunction with the medial and/or lateral portions 156, 158 of the cuneiform guide 150 may be configured to facilitate placement of the metatarsal guide 110 in a desired position by referencing known anatomical features of the topology of the medial cuneiform 202.

As shown in FIGS. 1-9, the medial portion 156 of the cuneiform guide 150 includes an engagement feature 174 which, as shown, includes a substantially circular feature extending from and positioned above (e.g., superior relative to) the medial portion 156. The engagement feature is shown to include a texture 176, shown in an exemplary embodiment as a plurality of ridges, which may be gripped or otherwise contacted by a physician when placing the cuneiform guide 150 in a desired position on the medial cuneiform 202 (or when performing other operations involving the cuneiform guide 150). In some aspects, the engagement feature 174 may have an alternate position about the cuneiform guide 150, for example extending from and/or above the lateral portion 158. Further, the cuneiform guide 150 may include multiple engagement features 174, for example extending from/above both the medial and lateral portions 156, 158.

As shown in FIGS. 1-9, a shaft 160 is shown to extend upward from a central portion of the body 152 disposed substantially between the medial and lateral portions 156, 158. As shown, the shaft 160 includes a substantially oval or elliptically shaped cross-sectional geometry and is centered about a central axis extending substantially perpendicular to at least a portion of the surface of the medial cuneiform 202. A texture 164 is disposed on at least a portion of the outer surface of the shaft 160, shown in an exemplary embodiment as, for example, a plurality of ridges, so as to facilitate gripping of the shaft 160 by a physician or other medical personnel. The shaft 160 is shown to define a cavity 162 therein, where the cavity 162 establishes fluid communication between an upper portion of the shaft 160 and a lower portion of the shaft 160 (which abuts the lower surface 154) configured to contact or be positioned adjacent the surface of the medial cuneiform 202.

The system 100 further includes a wire guide 166 configured to facilitate the releasable coupling of the cuneiform guide 150 with the medial cuneiform 202. As shown in FIGS. 1-2, the wire guide 166 may be the same as or similar (e.g., size, dimensions, etc.) as the wire guide 126 shown to be releasably coupled with the metatarsal guide 110. The wire guide 166 includes a substantially oval or elliptical prism-shaped geometry (and accordingly a similarly shaped cross-sectional geometry). At least a portion of the wire guide 166 (as shown, the lower portion) is configured to have a complementary geometry to that of the cavity 162 (with a lesser lateral dimension) such that the portion of the wire guide 166 may be received by the cavity 162 therein. Further, at least a portion of the wire guide 166 is shown to have a greater lateral dimension than that of the cavity 162 (which may function as a depth stop feature) so as to limit the depth of insertion of the wire guide 166 within the cavity 162. The wire guide 166 is further shown to include at least one bore (which, as shown in at least FIGS. 1-3 includes a pair of bores and will be referred to hereinafter as such) 168 extending from a top surface of the wire guide 166 through a body portion thereof to a bottom surface of the wire guide 166. As shown in at least FIGS. 1-3, the bores 168 are centered about respective central axes and have a substantially cylindrical geometry. Further, the bores 168 are configured such that the central axes thereof are positioned substantially parallel to one another (although in some aspects, the bores 168 and central axes thereof may be angled so as to achieve converging, diverging, or other non-parallel, oblique trajectories). As shown, the bores 168 are each configured to receive one of the stabilization wires 172 therein and therethrough and accordingly may be sized (e.g., diametrically, length-wise, etc.) to accommodate one or more sizes of the stabilization wires 172. In some aspects, the system 100 may include a single wire guide 126 or 166, with the wire guide releasably coupled and decoupled with both the metatarsal guide 110 and the cuneiform guide 150 in performing a surgical method or procedure.

The cuneiform guide 150 may be implemented in conjunction with other components of the system 100 to couple the cuneiform guide 150 with the medial cuneiform 202 of the patient. The cuneiform guide 150 may first be positioned and placed by a physician such that at least a portion of the lower surface 154 interfaces with the surface of the medial cuneiform 202 of the patient. The cuneiform guide 150 may include one or more features, for example the bottom surface 154 and/or the medial and/or lateral portions 156, 158, that facilitate placement of the cuneiform guide 150 in a desired position (and may provide haptic feedback to a physician confirming proper placement/positioning of the guide 150). For example, the lower surface 154 and the medial portion 156 may collectively include a curvature configured to interface with a medial portion of the surface of the medial cuneiform 202. Further, the lower surface 154 and/or the lateral portion 158 of the cuneiform guide 150 may include an extension or other feature configured to wrap around a portion of the medial cuneiform 202 so as to interface with and reference an anatomical feature of the medial cuneiform 202.

Once the cuneiform guide 150 has been placed in a desired position, the wire guide 166 (or, in the instance of a single wire guide 126 or 166 being implemented in conjunction with the system 100, the wire guide 126) may be inserted such that at least a portion of the guide 150 is received by and positioned therein the cavity 162 of the cuneiform guide 150. In placing the wire guide 166 at least partially within the cavity 162, a physician may grip the cuneiform guide 150 via the texture 164 provided thereon, and/or may also grip the wire guide 166 by a texture 170 (shown in FIGS. 1-3 as a plurality of ridges) disposed on an outer surface thereof. Various features of the cuneiform guide 150, for example those of the lower surface 154 in conjunction with the medial and lateral portions 156, 158 as described previously herein may provide haptic feedback to a physician in that the one or more features retain the cuneiform guide 150 in a desired position when the feature(s) contact the corresponding complementary anatomical features to which they are contoured and referenced. In some aspects, a physician may elect to place at least a portion of the wire guide 166 within the cavity 162 prior to positioning the cuneiform guide 150 on the first metatarsal 206 of the patient. Once the cuneiform guide 150 is placed in a desired position and the wire guide 166 has been inserted at least partially within the cavity 162 of the cuneiform guide 150, one of the stabilization wires 172 may be placed at least partially within one of the bores 168 such that the pointed tip of the stabilization wire 172 is positioned adjacent the surface of the medial cuneiform 202. A physician may then couple the stabilization wire 172 with the medial cuneiform 202 using one or more instruments common to orthopedic surgical procedures, for example, a drill or other powered driver. In coupling the stabilization wire 172 with the medial cuneiform 202, the cuneiform guide 150 and the wire guide 166 are also releasably coupled with the first metatarsal 206. Once a first stabilization wire 172 has been placed, a second stabilization wire 172 may also be placed according to the same procedure. Following placement of the pair of stabilization wires 172, the wire guide 166 and/or the cuneiform guide 150 may be decoupled from the medial cuneiform 202 with the stabilization wires 172 remaining coupled with the medial cuneiform 202.

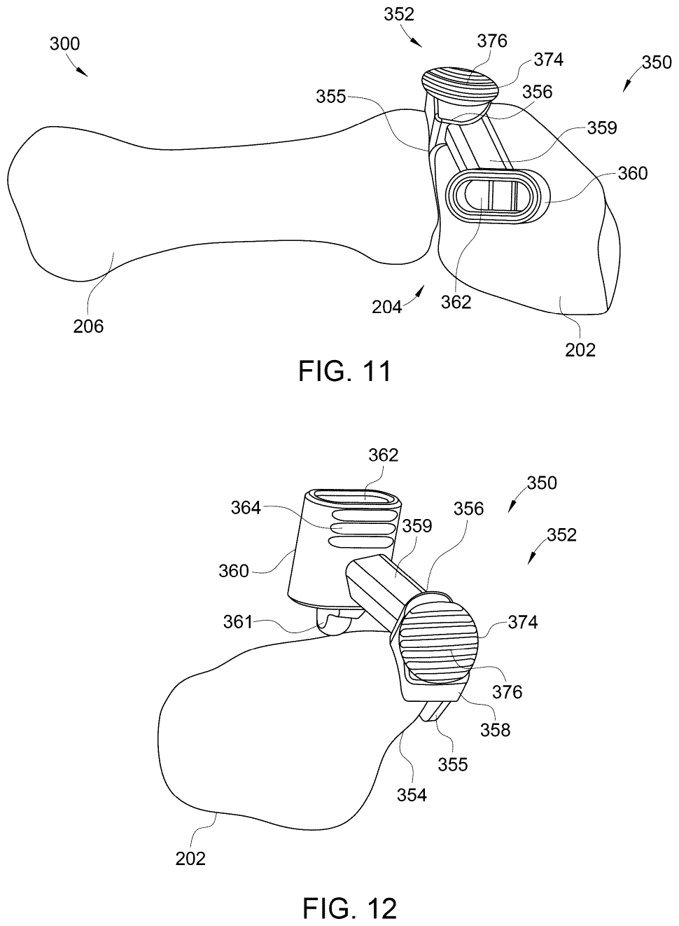

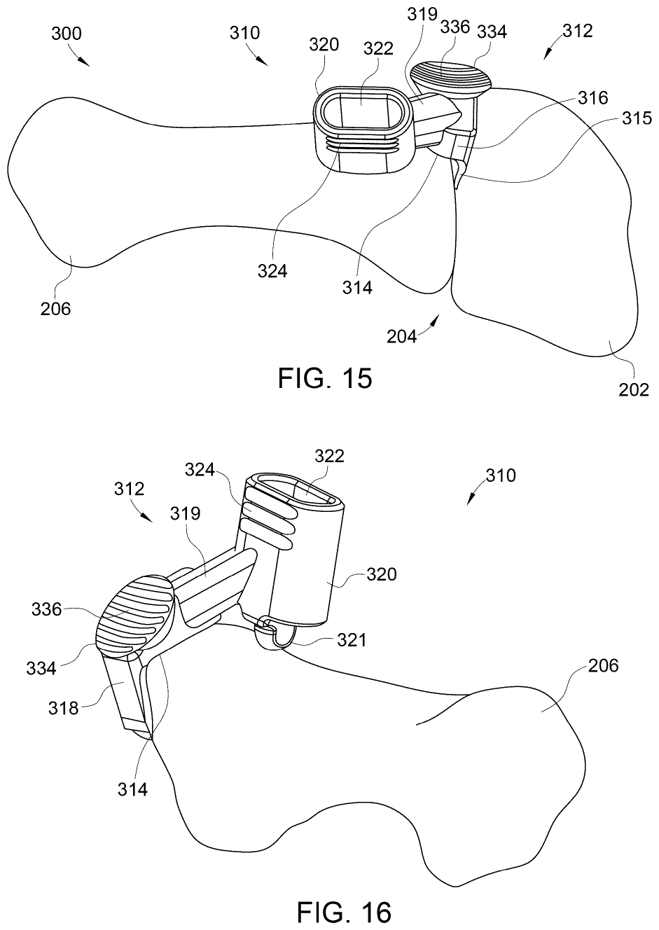

Referring now to FIGS. 10-17, there is illustrated an exemplary embodiment of a system 300 for performing at least a portion of a Lapidus joint procedure. In some aspects, the system 300 may be implemented in conjunction with other components common to orthopedic procedures, for example, drills, reciprocating/sagittal saws, and other powered instruments which, for the sake of brevity, are not shown herein. The system 300 as shown is a patient-specific system and, accordingly, one or more components or features thereof may correspond to a specific portion of a specific anatomy of a specific patient. For example, a component in the system 300 may include a surface that is complementary in concavity to a convexity of a portion of a bone of a patient with which the portion of the component may interface. In some aspects, one or more components of the system 300 may be generated based on analysis of an input that includes patient data, for example, patient imaging data including, for example, a CT or X-Ray data.

The system 300 includes a metatarsal guide 310 configured to interface and releasably couple with a first metatarsal 206 of a patient, as well as a cuneiform guide 350 configured to interface and releasably couple with a medial cuneiform 202 of a patient as shown in at least FIGS. 10 and 14. As shown, the first metatarsal guide 310 is configured to interface with a proximal portion (e.g., the base) of the first metatarsal 206, whereas the cuneiform guide 350 is configured to interface with a distal portion of the medial cuneiform 202. In some aspects, one or both of the guides 310, 350 may include one or more features which, when the guide 310, 350 is placed in a desired position on the appropriate anatomy, extend into or near a joint space 204 of the metatarsocuneiform joint (e.g., the Lapidus joint). Each of the guides 310, 350 are shown to be releasably couplable with the first metatarsal 206 and the medial cuneiform 202, respectively. In some aspects, the guides 310, 350 may include features to facilitate releasable coupling therebetween, for example, a dovetail or other similar features. In some aspects, the guides 310, 350 may be configured to be releasably coupled with the first metatarsal 206 and the medial cuneiform 202, respectively, simultaneously, while in some aspects the guides 310, 350 may be configured to be coupled and decoupled from their respective anatomy in a sequential fashion.

The metatarsal guide 310 is shown to include a body 312, wherein the body 312 includes a medial portion 316 substantially opposite the guide 310 from a lateral portion 318. As shown, the body 312 includes a lower surface 314 (at least a portion of which is configured to interface with a surface of the first metatarsal 206) extending from the medial portion 316 to the lateral portion 318. The lower surface 314 may be a partial surface, for example including one or more concavities and/or recesses therein. In some aspects, the lower surface 314 may include one or more curvatures, concavities, convexities, or changes in elevation configured to accommodate a surface of the first metatarsal 206 and/or various features of the metatarsal guide 310 configured to facilitate the placement and coupling of the metatarsal guide 310 with the first metatarsal 206. In some aspects, the lower surface 314 may include a single plane (or multiple planes) configured to contact a specific portion or surface of the first metatarsal 206.

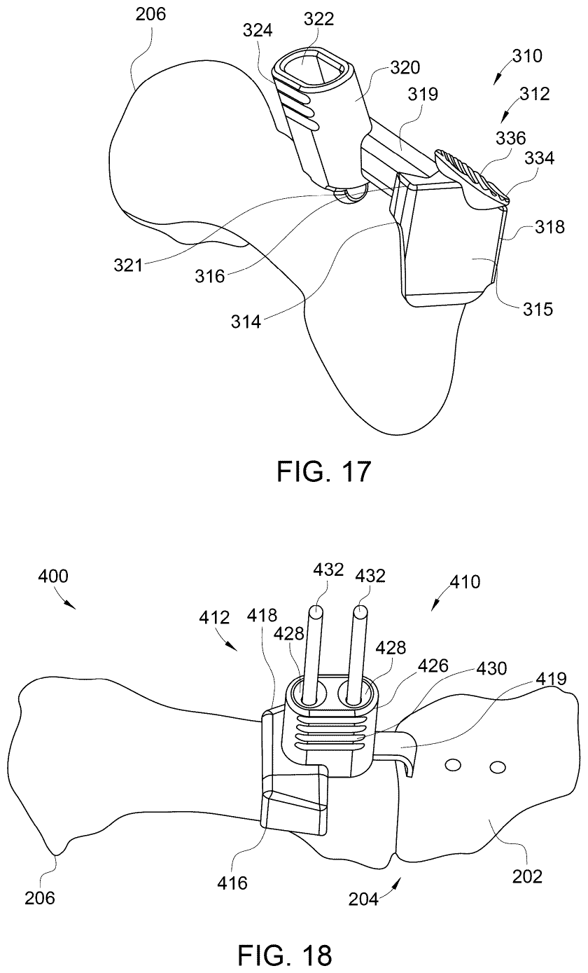

As shown in FIGS. 16-17, the lateral portion 318 includes a positioning feature which may include an angulation (e.g., two planes arranged in an oblique or orthogonal configuration) or curvature (e.g., including a radiused or otherwise arcuate portion) disposed and/or extending between a first plane of the lower surface 314 (which may be substantially parallel with a portion of the surface of the first metatarsal 206, or positioned at an oblique angle relative to the portion of the surface) and a second plane of the lower surface 314 (which may be oblique or substantially perpendicular relative to the first plane). In the previous example, the radiused or arcuate portion of the lower surface 314 may be contoured to a curvature of the first metatarsal 206 of the patient. The lateral portion 318 may include a curved geometry in order to provide structural support for the contouring of the metatarsal guide 310. This feature may generally take the form of a “hook” shape or other similar geometry and extend around the top portion of the first metatarsal 206 to contact a “flat” anatomical feature found on the lateral portion of the anatomy of the first metatarsal 206. The medial portion 316 may include a similar feature to that of the lateral portion 318 which, in conjunction with the lower surface 314, provides structural support for a contouring feature of the lower surface 314 that is configured to contact one or more portions of the medial anatomy of the first metatarsal 206. The structural features of the lower surface 314 in conjunction with the medial and/or lateral portions 316, 318 of the metatarsal guide 310 may be configured to facilitate placement of the metatarsal guide 310 in a desired position by referencing known anatomical features of the topology of the first metatarsal 206.

As shown in FIGS. 14-17, the lateral portion 318 of the metatarsal guide 310 includes an engagement feature 334 which, as shown, includes a substantially circular feature extending from and positioned above (e.g., superior relative to) the lateral portion 318. The engagement feature is shown to include a texture 336, shown in an exemplary embodiment, for example, as a plurality of ridges, which may be gripped or otherwise held by a physician when placing the metatarsal guide 310 in a desired position on the first metatarsal 206 (or when performing other operations involving the metatarsal guide 310). In some aspects, the engagement feature 334 may have an alternate position about the metatarsal guide 310, for example extending from and/or above the medial portion 316. Further, the metatarsal guide 310 may include multiple engagement features 334, for example extending from/above both the medial and lateral portions 316, 318. The metatarsal guide 310 is further shown to include a paddle 315 extending in a downward direction from the lower surface 314 and extending from the medial portion 316 to the lateral portion 318. As shown in FIG. 17, the paddle 315 is configured to be positioned at least partially within the joint space 204 and abut at least a proximal surface or point thereon of the first metatarsal 206.

An arm 319 is shown to extend (both laterally and upward, in an anatomically medial-dorsal direction when the metatarsal guide 310 is coupled with the first metatarsal 206) from the body 312, with the arm 319 including a shaft 320 at its terminal end. The shaft 320 is shown to extend upward from the arm 319 and includes a footprint that is offset from that of the body 312. As shown, the shaft 320 includes a substantially oval or elliptically shaped cross-sectional geometry and is centered about a central axis extending substantially perpendicular to at least a portion of the surface of the first metatarsal 206. A texture 324 is disposed on at least a portion of the outer surface of the shaft 320, shown in an exemplary embodiment, for example, as a plurality of ridges, so as to facilitate gripping of the shaft 320 by a physician or other medical personnel. The shaft 320 is shown to define a cavity 322 therein, where the cavity 322 establishes fluid communication between an upper portion of the shaft 320 and a lower portion of the shaft 320 configured to be positioned adjacent the surface of the first metatarsal 206. In some aspects, the shaft 320 may include a crossing member positioned at the bottom portion of the shaft 320, with the crossing member extending from a first edge of the shaft 320, across the cavity 322, and to a second edge of the shaft 320. The shaft 320 further includes an engagement feature 321 extending downward from a bottom-most portion of the shaft 320 which, as shown, includes an at least partially rounded geometry. The engagement feature 321 may be configured to contact a portion of the surface of the first metatarsal 206 so as to provide reference for placement of the guide 310 as well as additional stability for the metatarsal guide 310 when placed in a desired position (e.g., establishing a third point of contact for the metatarsal guide 310 so as to establish a stabilization plane including the engagement feature 321 as well as points of contact between the surface of the first metatarsal 206 and the paddle 315 and the lower surface 314).

The system 300 further includes the wire guide 126 configured to facilitate the releasable coupling of the metatarsal guide 310 with the first metatarsal 206. The wire guide 126 is shown to include a substantially oval or elliptical prism-shaped geometry (and accordingly a similarly-shaped cross-sectional geometry). At least a portion of the wire guide 126 (as shown, the lower portion) is configured to have a complementary geometry to that of the cavity 322 (with a lesser lateral dimension) such that the portion of the wire guide 126 may be received by the cavity 322 therein. Further, at least a portion of the wire guide 126 is shown to have a greater lateral dimension than that of the cavity 322 (which may function as a depth stop feature) so as to limit the depth of insertion of the wire guide 126 within the cavity 322. The wire guide 126 is further shown to include at least one bore (which, as shown in at least FIG. 14 includes a pair of bores and will be referred to hereinafter as such) 128 extending from a top surface of the wire guide 126 through a body portion thereof to a bottom surface of the wire guide 126. As shown in at least FIGS. 10 and 14, the bores 128 are centered about respective central axes and have a substantially cylindrical geometry. Further, the bores 128 are configured such that the central axes thereof are positioned substantially parallel to one another (although in some aspects, the bores 128 and central axes thereof may be angled so as to achieve converging, diverging, or other non-parallel, oblique trajectories). As shown, the bores 128 are each configured to receive one of the stabilization wires 132 therein and therethrough and accordingly may be sized (e.g., diametrically, length-wise, etc.) to accommodate one or more sizes of the stabilization wires 132.

The metatarsal guide 310 may be implemented in conjunction with other components of the system 300 to couple the metatarsal guide 310 with the first metatarsal 206 of the patient. The metatarsal guide 310 may first be positioned and placed by a physician such that at least a portion of the lower surface 314 interfaces with the surface of the first metatarsal 206 of the patient. The metatarsal guide 310 may further be positioned such that one or more additional features, for example the paddle 315 and the engagement feature 321, facilitate placement of the metatarsal guide 310 in a desired position (and may provide haptic feedback to a physician confirming proper placement/positioning of the guide). Further, the lower surface 314 and the lateral portion 318 may collectively include a curvature configured to interface with a portion of the surface of the first metatarsal 206. In some aspects, the lower surface and/or the medial portion 316 of the metatarsal guide 310 may include an extension or other feature configured to wrap around a portion of the first metatarsal 206 so as to interface with and reference an anatomical feature of the first metatarsal 206.

Once the metatarsal guide 310 has been placed in a desired position, the wire guide 126 may be inserted such that at least a portion of the guide 310 is received by and positioned therein the cavity 322 of the metatarsal guide 310. In placing the wire guide 126 at least partially within the cavity 322, a physician may grip the metatarsal guide 310 via the texture 324 provided thereon, and/or may also grip the wire guide 126 by the texture 130 (shown in FIG. 14 as a plurality of ridges) disposed on an outer surface thereof. Various features of the metatarsal guide 310, for example those of the lower surface 314 in conjunction with the medial and lateral portions 316, 318, as well as the paddle 315 and the engagement feature 321 may provide haptic feedback to a physician in that the one or more features retain the metatarsal guide 310 in a desired position when the feature(s) contact the corresponding complementary anatomical features to which they are contoured and referenced. In some aspects, a physician may elect to place at least a portion of the wire guide 126 within the cavity 322 prior to positioning the metatarsal guide 310 on the first metatarsal 206 of the patient. Once the metatarsal guide 310 is placed in a desired position and the wire guide 126 has been inserted at least partially within the cavity 322 of the metatarsal guide 310, one of the stabilization wires 132 may be placed at least partially within one of the bores 128 such that the pointed tip of the stabilization wire 132 is positioned adjacent the surface of the first metatarsal 206. A physician may then couple the stabilization wire 132 with the first metatarsal 206 using one or more instruments common to orthopedic surgical procedures, for example, a drill or other powered driver. In coupling the stabilization wire 132 with the first metatarsal 206, the metatarsal guide 310 and the wire guide 126 are also releasably coupled with the first metatarsal 206. Once a first stabilization wire 132 has been placed, a second stabilization wire 132 may also be placed according to the same procedure. Following the placement of the pair of stabilization wires 132, the wire guide 126 and/or the metatarsal guide 310 may be decoupled from the first metatarsal 206 with the stabilization wires 132 remaining coupled with the first metatarsal 206.

The cuneiform guide 350 is shown to include a body 352, wherein the body includes a medial portion 356 substantially opposite the guide 350 from a lateral portion 358. As shown, the body 352 includes a lower surface 354 (at least a portion of which is configured to interface with a surface of the medial cuneiform 202) extending from the medial portion 356 to the lateral portion 358. The lower surface 354 may be a partial surface, for example including one or more concavities and/or recesses therein. In some aspects, the lower surface 354 may include one or more curvatures, concavities, convexities, or changes in elevation configured to accommodate a surface of the medial cuneiform 202 and/or various features of the cuneiform guide 350 configured to facilitate placement and coupling of the cuneiform guide 350 with the medial cuneiform 202. In some aspects, the lower surface 354 may include a single plane (or multiple planes) configured to contact a specific portion or surface of the medial cuneiform 202.

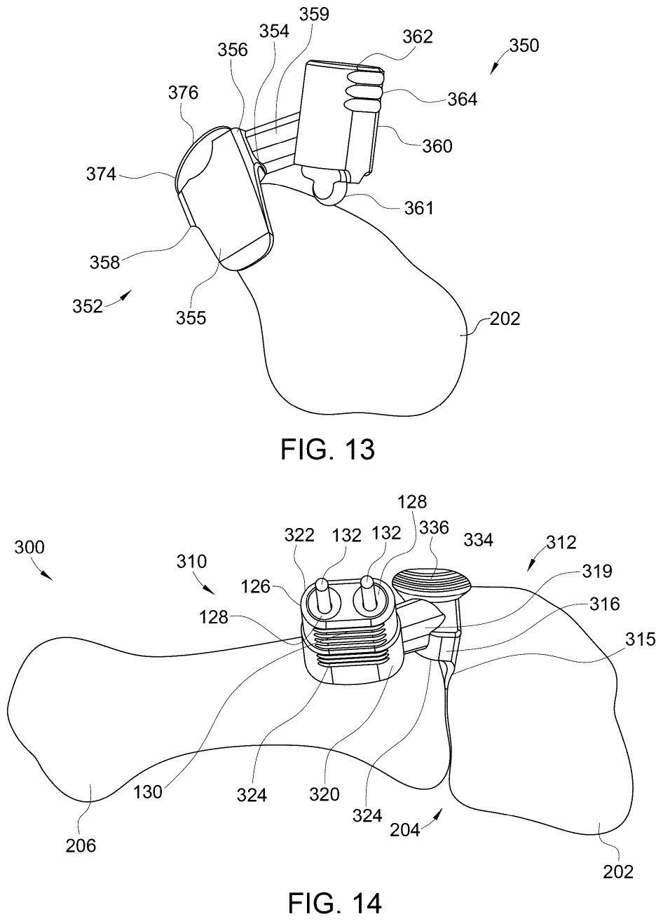

As shown in FIGS. 12-13, the lateral portion 358 includes a positioning feature which may include an angulation (e.g., two planes arranged in an oblique or orthogonal configuration) or curvature (e.g., including a radiused or otherwise arcuate portion) disposed and/or extending between a first plane of the lower surface 354 (which may be substantially parallel with a portion of the surface of the medial cuneiform 202, or positioned at an oblique angle relative to the portion of the surface) and a second plane of the lower surface 354 (which may be oblique or substantially perpendicular relative to the first plane). In the previous example, the radiused or arcuate portion of the lower surface 354 may be contoured to a curvature of the medial cuneiform 202 of the patient. The lateral portion 358 may include a curved geometry in order provide structural support for the contouring of the cuneiform guide 350. This feature may generally take the form of a “hook” shape or other similar geometry and extend around the top portion of the medial cuneiform 202 to contact a “flat” anatomical feature found on the lateral portion of the anatomy of the medial cuneiform 202. The medial portion 356 may include a similar feature to that of the lateral portion 358 which, in conjunction with the lower surface 354, provides structural support for a contouring feature of the lower surface 354 that is configured to contact one or more portions of the dorsal and/or medial anatomy of the first metatarsal 206. The structural features of the lower surface 354 in conjunction with the medial and/or lateral portions 356, 358 of the cuneiform guide 350 may be configured to facilitate placement of the metatarsal guide 310 in a desired position by referencing known anatomical features of the topology of the medial cuneiform 202.

As shown in FIGS. 10-13, the lateral portion 358 of the cuneiform guide 350 includes an engagement feature 374 which, as shown, includes a substantially circular feature extending from and positioned above (e.g., superior relative to) the lateral portion 358. The engagement feature is shown to include a texture 376, shown in an exemplary embodiment, for example, as a plurality of ridges, which may be gripped or otherwise held by a physician when placing the cuneiform guide 350 in a desired position on the medial cuneiform 202 (or when performing other operations involving the cuneiform guide 350). In some aspects, the engagement feature 374 may have an alternate position about the cuneiform guide 350, for example extending from and/or above the medial portion 356. Further, the cuneiform guide 350 may include multiple engagement features 374, for example extending from/above both the medial and lateral portions 356, 358. The cuneiform guide 350 is further shown to include a paddle 355 extending in a downward direction from the lower surface 354 and extending from the medial portion 356 to the lateral portion 358. As shown in FIG. 17, the paddle 355 is configured to be positioned at least partially within the joint space 204 and abut at least a proximal surface or point thereon of the medial cuneiform 202.

An arm 359 is shown to extend (both laterally and upward, in an anatomically medial-dorsal direction when the cuneiform guide 350 is coupled with the medial cuneiform 202) from the body 352, with the arm 359 including a shaft 360 at its terminal end. The shaft 360 is shown to extend upward from the arm 359 and includes a footprint that is offset from that of the body 352. As shown, the shaft 360 includes a substantially oval or elliptically shaped cross-sectional geometry and is centered about a central axis extending substantially perpendicular to at least a portion of the surface of the medial cuneiform 202. A texture 364 is disposed on at least a portion of the outer surface of the shaft 360, shown in an exemplary embodiment as, for example, a plurality of ridges, so as to facilitate gripping of the shaft 360 by a physician or other medical personnel. The shaft 360 is shown to define a cavity 362 therein, where the cavity 362 establishes fluid communication between an upper portion of the shaft 360 and a lower portion of the shaft 360 configured to be positioned adjacent the surface of the medial cuneiform 202. In some aspects, the shaft 360 may include a crossing member positioned at the bottom portion of the shaft 360, with the crossing member extending from a first edge of the shaft 360, across the cavity 362, and to a second edge of the shaft 360. The shaft 360 further includes an engagement feature 321 extending downward from a bottom-most portion of the shaft 360 which, as shown, includes an at least partially rounded geometry. The engagement feature 321 may be configured to contact a portion of the surface of the medial cuneiform 202 so as to provide a reference for placement of the guide as well as additional stability for the cuneiform guide 350 when placed in a desired position (e.g., establishing a third point of contact for the cuneiform guide 350 so as to establish a stabilization plane including the engagement feature 321 as well as points of contact between the surface of the medial cuneiform 202 and the paddle 355 and the lower surface 354).

The system 300 further includes the wire guide 126 (the same as or similar to that shown and described previously herein) configured to facilitate the releasable coupling of the cuneiform guide 350 with the medial cuneiform 202. The wire guide 126 is shown to include a substantially oval or elliptical prism-shaped geometry (and accordingly a similarly shaped cross-sectional geometry). At least a portion of the wire guide 126 (as shown, the lower portion) is configured to have a complementary geometry to that of the cavity 362 (with a lesser lateral dimension) such that the portion of the wire guide 126 may be received by the cavity 362 therein. Further, at least a portion of the wire guide 126 is shown to have a greater lateral dimension than that of the cavity 362 (which may function as a depth stop feature) so as to limit the depth of insertion of the wire guide 126 within the cavity 362. The wire guide 126 is further shown to include at least one bore (which, as shown in at least FIG. 10 includes a pair of bores and will be referred to hereinafter as such) 128 extending from a top surface of the wire guide 126 through a body portion thereof to a bottom surface of the wire guide 126. As shown in at least FIGS. 10 and 14, the bores 128 are centered about respective central axes and have a substantially cylindrical geometry. Further, the bores 128 are configured such that the central axes thereof are positioned substantially parallel to one another (although in some aspects, the bores 128 and central axes thereof may be angled so as to achieve converging, diverging, or other non-parallel, oblique trajectories). As shown, the bores 128 are each configured to receive one of the stabilization wires 132 therein and therethrough and accordingly may be sized (e.g., diametrically, length-wise, etc.) to accommodate one or more sizes of the stabilization wires 132.

The cuneiform guide 350 may be implemented in conjunction with other components of the system 300 to couple the cuneiform guide 350 with the medial cuneiform 202 of the patient. The cuneiform guide 350 may first be positioned and placed by a physician such that at least a portion of the lower surface 354 interfaces with the surface of the medial cuneiform 202 of the patient. The cuneiform guide 350 may further be positioned such that one or more additional features, for example the paddle 355 and the engagement feature 321, facilitate placement of the cuneiform guide 350 in a desired position (and may provide haptic feedback to a physician confirming proper placement/positioning of the guide). Further, the lower surface 354 and the lateral portion 358 may collectively include a curvature configured to interface with a portion of the surface of the medial cuneiform 202. In some aspects, the lower surface and/or the medial portion 356 of the cuneiform guide 350 may include an extension or other feature configured to wrap around a portion of the medial cuneiform 202 so as to interface with and reference an anatomical feature of the medial cuneiform 202.

Once the cuneiform guide 350 has been placed in a desired position, the wire guide 126 may be inserted such that at least a portion of the guide 350 is received by and positioned therein the cavity 362 of the cuneiform guide 350. In placing the wire guide 126 at least partially within the cavity 362, a physician may grip the cuneiform guide 350 via the texture 364 provided thereon, and/or may also grip the wire guide 126 by the texture 130 (shown in FIG. 10 as a plurality of ridges) disposed on an outer surface thereof. Various features of the cuneiform guide 350, for example those of the lower surface 354 in conjunction with the medial and lateral portions 356, 358, as well as the paddle 355 and the engagement feature 321 may provide haptic feedback to a physician in that the one or more features retain the cuneiform guide 350 in a desired position when the feature(s) contact the corresponding complementary anatomical features to which they are contoured and reference. In some aspects, a physician may elect to place at least a portion of the wire guide 126 within the cavity 362 prior to positioning the cuneiform guide 350 on the first metatarsal 206 of the patient. Once the cuneiform guide 350 is placed in a desired position and the wire guide 126 has been inserted at least partially within the cavity 362 of the cuneiform guide 350, one of the stabilization wires 132 may be placed at least partially within one of the bores 128 such that the pointed tip of the stabilization wire 132 is positioned adjacent the surface of the medial cuneiform 202. A physician may then couple the stabilization wire 132 with the medial cuneiform 202 using one or more instruments common to orthopedic surgical procedures, for example, a drill or other powered driver. In coupling the stabilization wire 132 with the medial cuneiform 202, the cuneiform guide 350 and the wire guide 126 are also releasably coupled with the first metatarsal. Once a first stabilization wire 132 has been placed, a second stabilization wire 132 may also be placed according to the same procedure. Following placement of the pair of stabilization wires 132, the wire guide 126 and/or the cuneiform guide 350 may be decoupled from the medial cuneiform 202 with the stabilization wires 132 remaining coupled with the medial cuneiform 202.

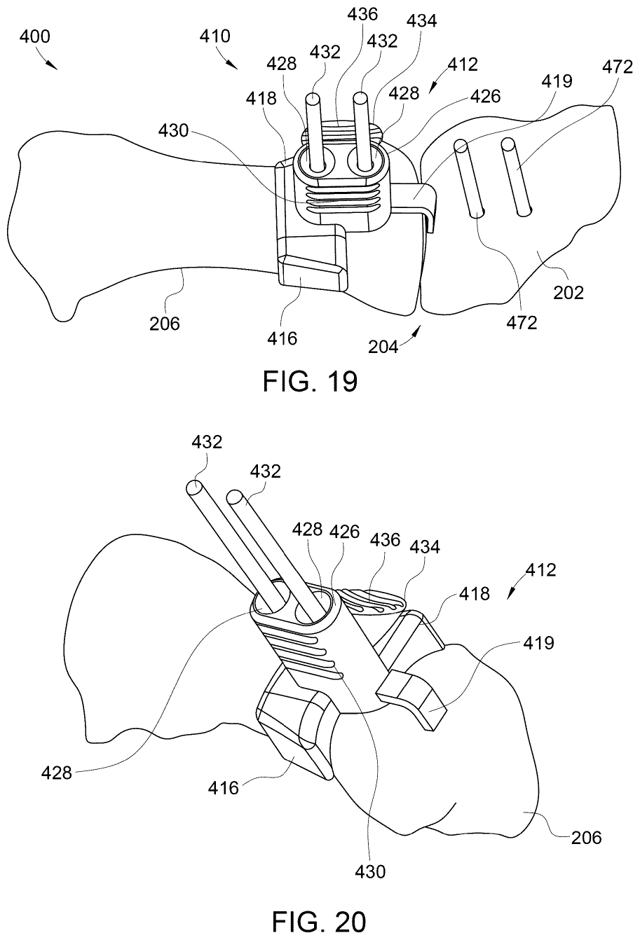

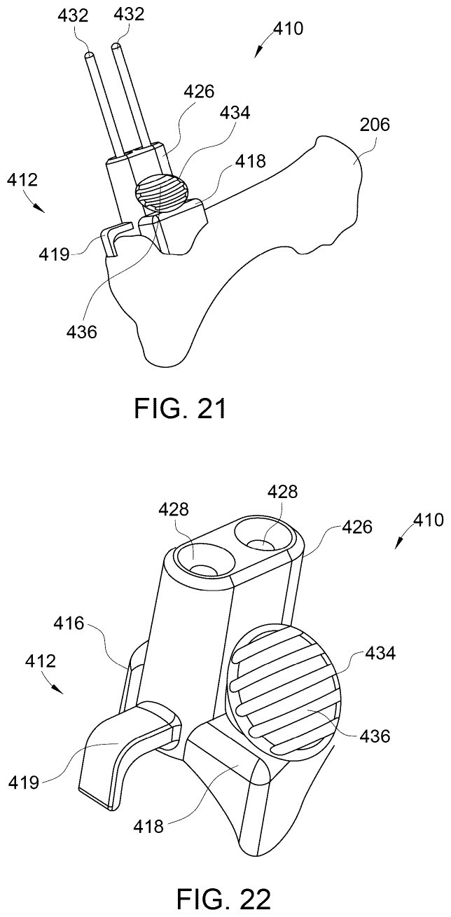

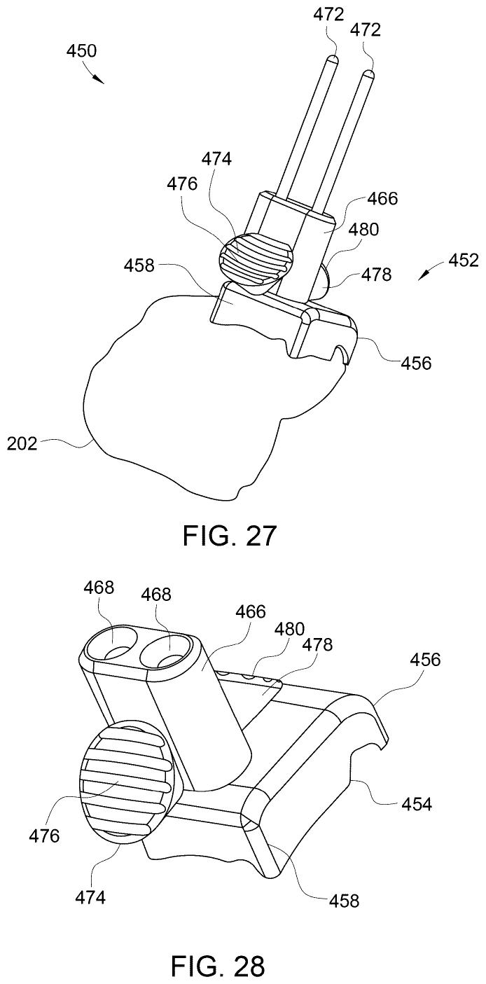

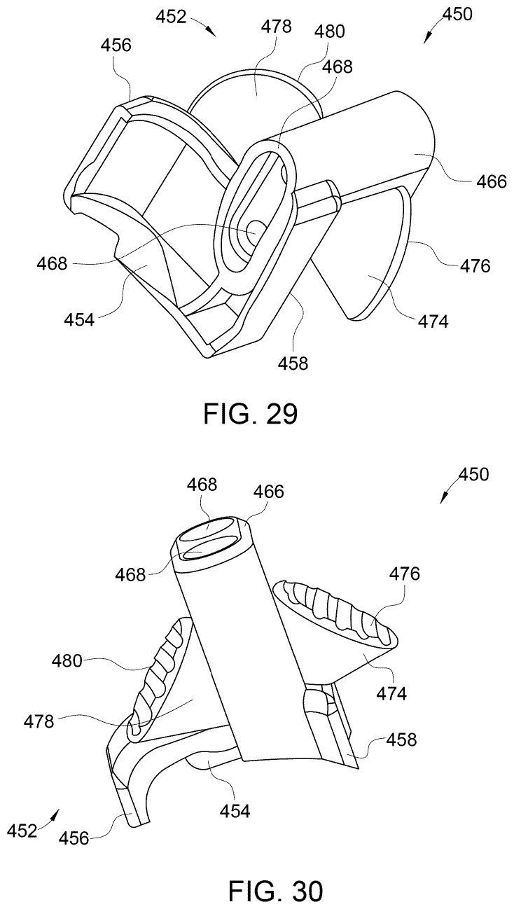

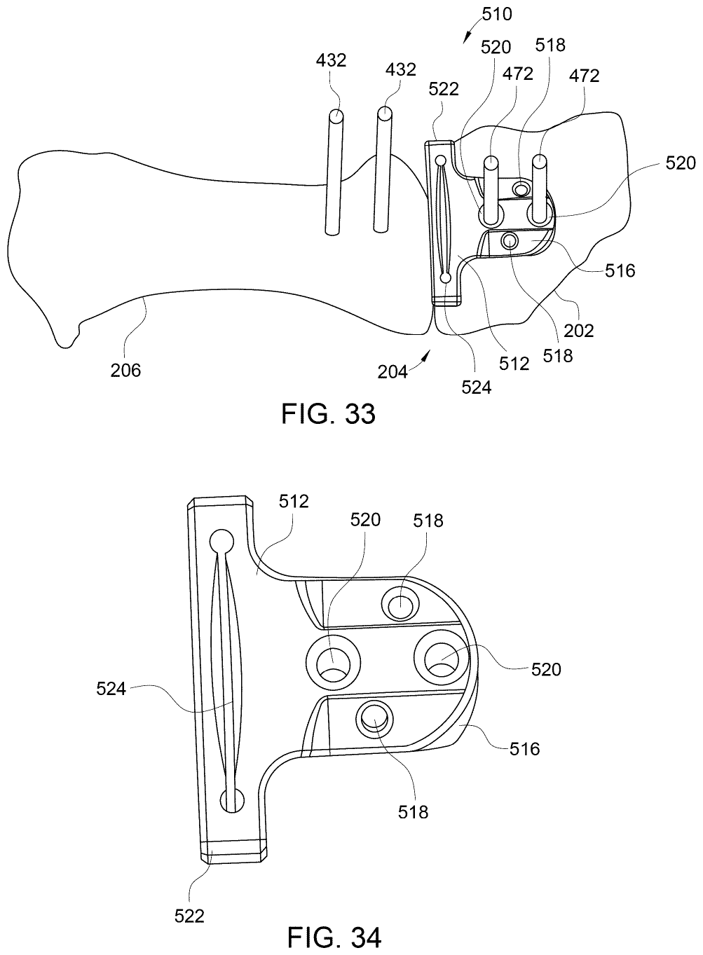

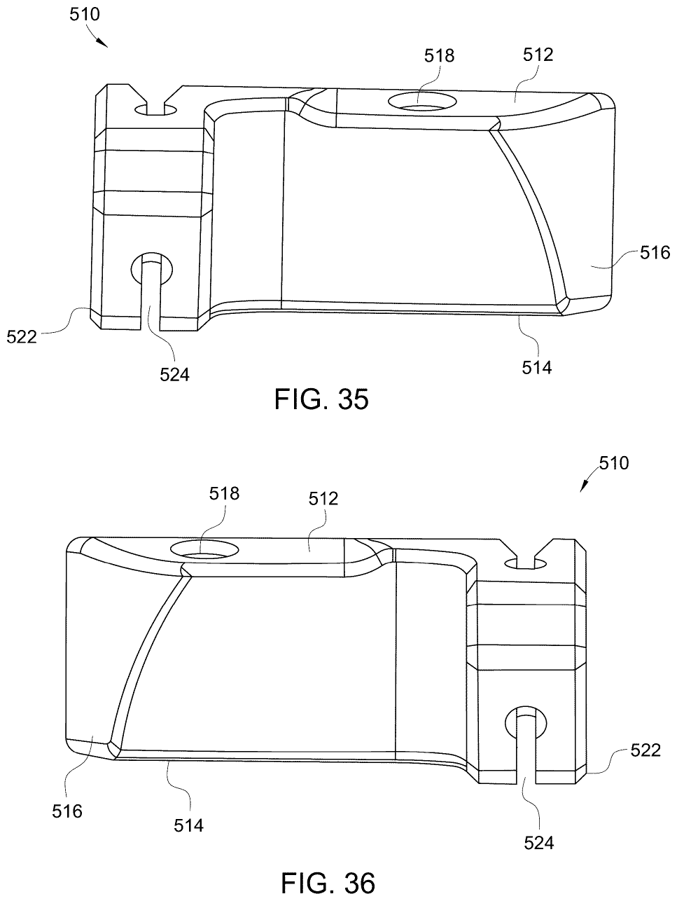

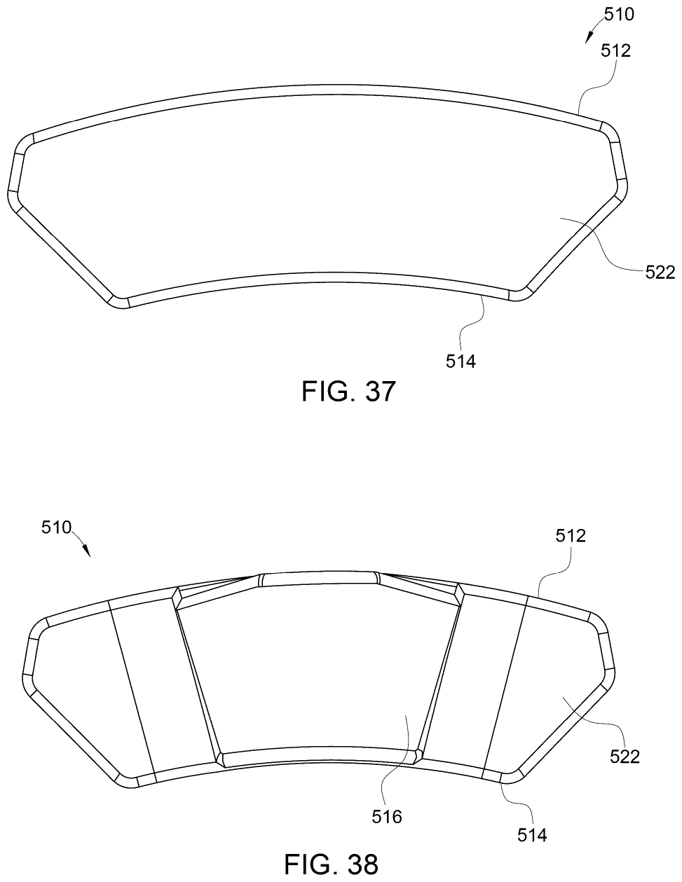

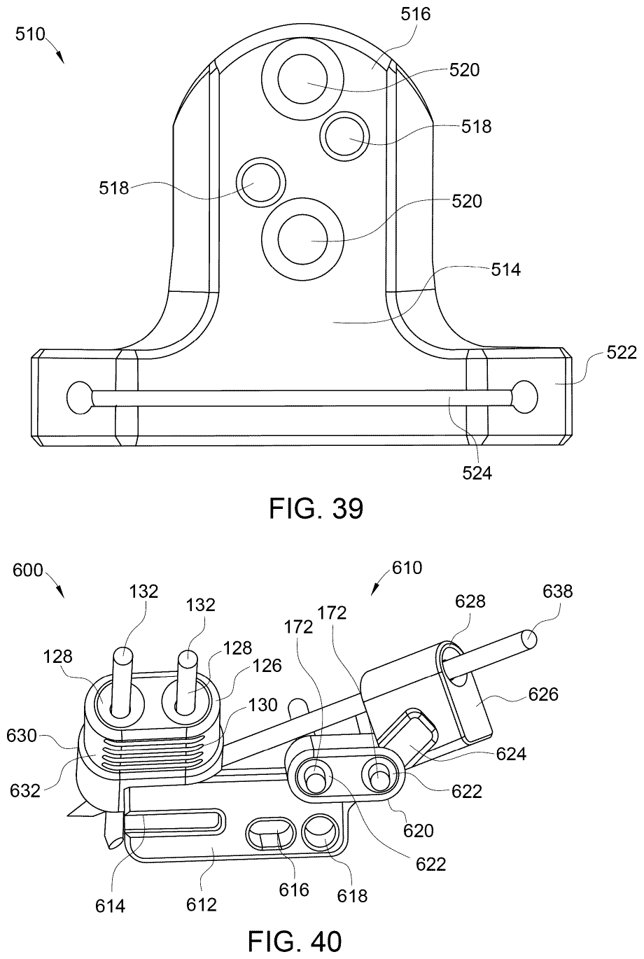

Referring now to FIGS. 18-39, there is illustrated an exemplary embodiment of a system 400 for performing at least a portion of a Lapidus joint procedure. In some aspects, the system 400 may be implemented in conjunction with other components common to orthopedic procedures, for example drills, reciprocating/sagittal saws, and other powered instruments which, for the sake of brevity, are not shown herein. The system 400 as shown is a patient-specific system and, accordingly, one or more components or features thereof may correspond to a specific portion of a specific anatomy of a specific patient. For example, a component in the system 400 may include a surface that is complementary in concavity to a convexity of a portion of a bone of a patient with which the portion of the component may interface. In some aspects, one or more components of the system 400 may be generated based on analysis of an input that includes patient data, for example patient imaging data including, for example, a CT or X-Ray data.

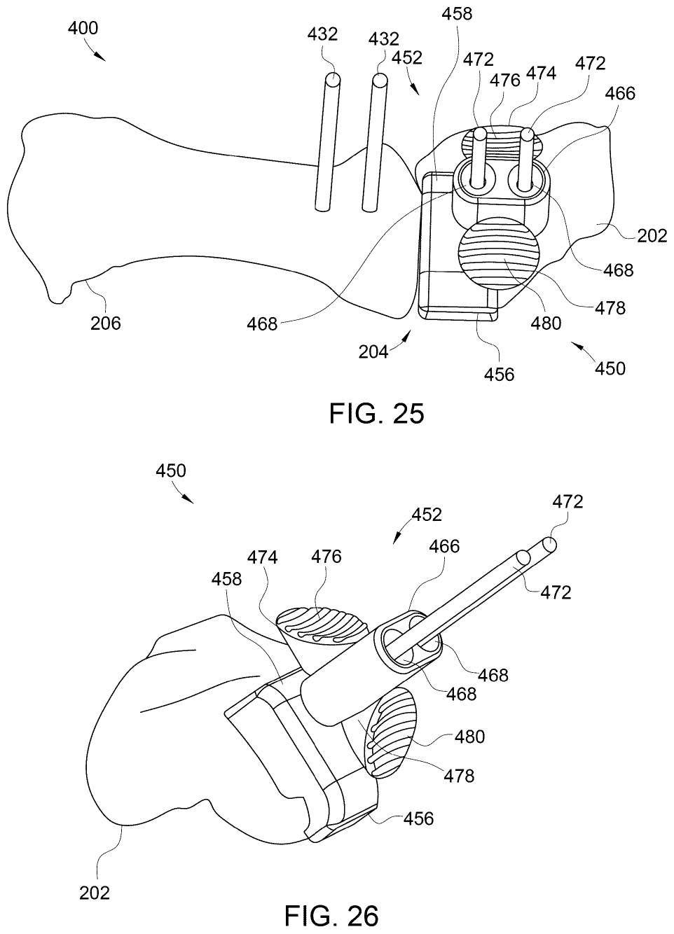

The system 400 includes a metatarsal guide 410 configured to interface and releasably couple with a first metatarsal 206 of a patient, as well as a cuneiform guide 450 configured to interface and releasably couple with a medial cuneiform 202 of a patient as shown in at least FIGS. 18 and 25. As shown, the first metatarsal guide 410 is configured to interface with a proximal portion (e.g., the base) of the first metatarsal 206, whereas the cuneiform guide 450 is configured to interface with a distal portion of the medial cuneiform 202. In some aspects, one or both of the guides 410, 450 may include one or more features which, when the guide is placed in a desired position on the appropriate anatomy, extend into or near a joint space 204 of the metatarsocuneiform joint (e.g., the Lapidus joint). Each of the guides 410, 450 are shown to be releasably couplable with the first metatarsal 206 and the medial cuneiform 202, respectively. In some aspects, the guides 410, 450 may include features to facilitate releasable coupling therebetween, for example, a dovetail or other similar features.

The metatarsal guide 410 is shown to include a body 412, wherein the body includes a medial portion 416 substantially opposite the guide 410 from a lateral portion 418. As shown, the body 412 includes a lower surface 414 (at least a portion of which is configured to interface with a surface of the first metatarsal 206) extending from the medial portion 416 to the lateral portion 418. The lower surface 414 may be a partial surface, for example including one or more concavities and/or recesses therein. In some aspects, the lower surface 414 may include one or more curvatures, concavities, convexities, or changes in elevation configured to accommodate a surface of the first metatarsal 206 and/or various features of the metatarsal guide 410 configured to facilitate placement and coupling of the metatarsal guide 410 with the first metatarsal 206.

As shown in FIGS. 18-24, the medial portion 416 includes a curvature which includes a radiused or otherwise arcuate portion extending between a first plane of the lower surface 414 that is substantially parallel with a portion of the surface of the first metatarsal 206 and a second plane of the lower surface 414 that is substantially perpendicular relative to the first plane. In the previous example, the radiused or arcuate portion of the lower surface 414 may be contoured to a curvature of the first metatarsal 206 of the patient. As shown, the lateral portion 418 includes a similarly curved geometry in order provide structural support for the contouring of the metatarsal guide 410. This feature may generally take the form of a “hook” shape or other similar geometry and extend around the top portion of the first metatarsal 206 to contact a “flat” anatomical feature found on the lateral portion of the anatomy of the first metatarsal 206. The medial portion 416 may include a similar feature to that of the lateral portion 418 which, in conjunction with the lower surface 414, provides structural support for a contouring feature of the lower surface 414 that is configured to contact one or more portions of the medial anatomy of the first metatarsal. The structural features of the lower surface 414 in conjunction with the medial and/or lateral portions 416, 418 of the metatarsal guide 410 may be configured to facilitate placement of the metatarsal guide in a desired position by referencing known anatomical features of the topology of the first metatarsal 206.

The lateral portion 418 of the metatarsal guide 410 includes an engagement feature 434 which, as shown, includes a substantially circular feature extending from and positioned above (e.g., superior relative to) the lateral portion 418. The engagement feature is shown to include a texture 436, shown in an exemplary embodiment, for example, as a plurality of ridges, which may be gripped or otherwise held by a physician when placing the metatarsal guide 410 in a desired position on the first metatarsal 206 (or when performing other operations involving the metatarsal guide 410). In some aspects, the engagement feature 434 may have an alternate position about the metatarsal guide 410, for example extending from and/or above the medial portion 416. Further, the metatarsal guide 410 may include multiple engagement features 434, for example extending from or above both the medial and lateral portions 416, 418.

The metatarsal guide 410 is shown to include a shaft 420 extending upward from a central portion of the body 412 disposed substantially between the medial and lateral portions 416, 418. In some aspects, at least a portion of the footprint of the shaft 420 may extend beyond that of the body 412. As shown, the shaft 420 includes a substantially oval or elliptically shaped cross-sectional geometry and is centered about a central axis extending substantially perpendicular to at least a portion of the surface of the first metatarsal 206. A texture 424 is disposed on at least a portion of the outer surface of the shaft 420, shown in an exemplary embodiment as, for example, a plurality of ridges, so as to facilitate gripping of the shaft 420 by a physician or other medical personnel. A lower portion of the shaft 420 (which abuts the lower surface 414) is configured to contact or be positioned adjacent the surface of the first metatarsal 206 when the metatarsal guide 410 is placed in a desired position. The shaft 420 includes an arm 419 extending laterally and downward therefrom such that, when the metatarsal guide 410 is placed in a desired position on the first metatarsal 206, the arm contacts at least a portion of the proximal-most aspect of the first metatarsal 206 and/or extends into the joint space 204.

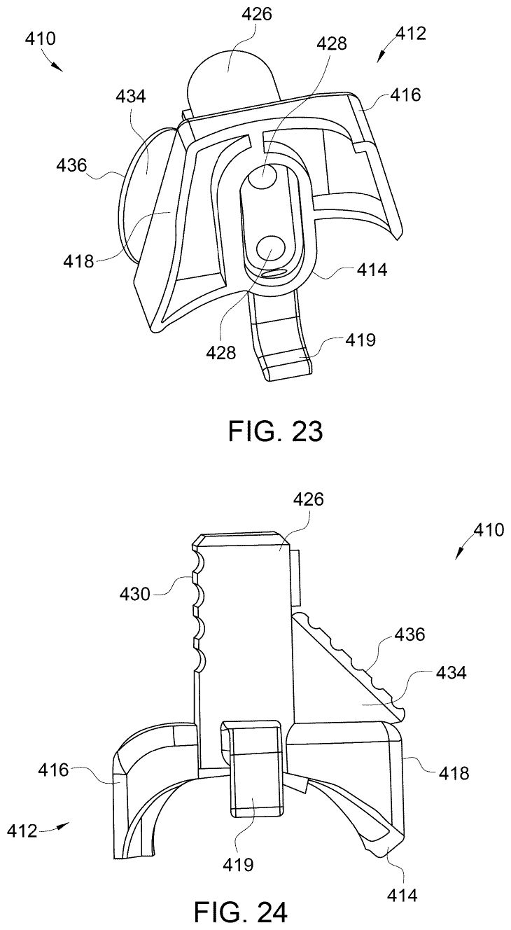

The system 400 further includes a wire guide 426 which is integral with the body 412 and shaft 420 and is configured to facilitate the releasable coupling of the metatarsal guide 410 with the first metatarsal 206. The wire guide 426 is shown to include a substantially oval or elliptical prism-shaped geometry (and accordingly a similarly shaped cross-sectional geometry), and in some aspects may include one or more features or dimensions the same as or similar to that of the wire guide 126 as shown and described previously herein. The wire guide 426 is further shown to include at least one bore (which, as shown in at least FIGS. 22-23 includes a pair of bores and will be referred to hereinafter as such) 428 extending from a top surface of the wire guide 426 through a body portion thereof to a bottom surface of the wire guide 426. As shown in at least FIGS. 22-23, the bores 428 are centered about respective central axes and have a substantially cylindrical geometry. Further, the bores 428 are configured such that the central axes thereof are positioned substantially parallel to one another (although in some aspects, the bores 428 and central axes thereof may be angled so as to achieve converging, diverging, or other non-parallel, oblique trajectories). As shown, the bores 428 are each configured to receive one of the stabilization wires 432 therein and therethrough and accordingly may be sized (e.g., diametrically, length-wise, etc.) to accommodate one or more sizes of the stabilization wires 432.

The metatarsal guide 410 may be implemented in conjunction with other components of the system 400 to couple the metatarsal guide 410 with the first metatarsal 206 of the patient. The metatarsal guide 410 may first be positioned and placed by a physician such that at least a portion of the lower surface 414 interfaces with the surface of the first metatarsal 206 of the patient. The metatarsal guide 410 may include one or more features, for example the bottom surface 414 and/or the medial and/or lateral portions 416, 418, that facilitate placement of the metatarsal guide 410 in a desired position (and may provide haptic feedback to a physician confirming proper placement/positioning of the guide 410). For example, the lower surface 414 and the medial portion 416 may collectively include a curvature configured to interface with a medial portion of the surface of the first metatarsal 206. Further, the lower surface and/or the lateral portion 418 of the metatarsal guide 410 mayp0 include an extension or other feature configured to wrap around a portion of the first metatarsal 206 so as to interface with and reference an anatomical feature of the first metatarsal 206.

In placing the metatarsal guide 410 in a desired position, a physician may grip the metatarsal guide 410 via the texture 424 provided thereon, and/or may also grip the wire guide 426 by a texture 430 (shown in FIGS. 22-23 as a plurality of ridges) disposed on an outer surface thereof. Various features of the metatarsal guide 410, for example, those of the lower surface 414 in conjunction with the medial and lateral portions 416, 418 as described previously herein may provide haptic feedback to a physician in that the one or more features retain the metatarsal guide 410 in a desired position when the feature(s) contact the corresponding complementary anatomical features to which they are contoured and referenced. Once the metatarsal guide 410 is placed in a desired position, one of the stabilization wires 432 may be placed at least partially within one of the bores 428 such that the pointed tip of the stabilization wire 432 is positioned adjacent the surface of the first metatarsal 206. A physician may then couple the stabilization wire 432 with the first metatarsal 206 using one or more instruments common to orthopedic surgical procedures, for example, a drill or other powered driver. In coupling the stabilization wire 432 with the first metatarsal 206, the metatarsal guide 410 is also releasably coupled with the first metatarsal 206. Once a first stabilization wire 432 has been placed, a second stabilization wire 432 may also be placed according to the same procedure. Following placement of the pair of stabilization wires 432, the metatarsal guide 410 may be decoupled from the first metatarsal 206 with the stabilization wires 432 remaining coupled with the first metatarsal 206.

Referring now to FIGS. 25-30, the cuneiform guide 450 is shown to include a body 452, wherein the body includes a medial portion 456 substantially opposite the guide 450 from a lateral portion 458. As shown, the body 452 includes a lower surface 454 (at least a portion of which is configured to interface with a surface of the medial cuneiform 202) extending from the medial portion 456 to the lateral portion 458. The lower surface 454 may be a partial surface, for example, including one or more concavities and/or recesses therein. In some aspects, the lower surface 454 may include one or more curvatures, concavities, convexities, or changes in elevation configured to accommodate a surface of the medial cuneiform 202 and/or various features of the cuneiform guide 450 configured to facilitate placement and coupling of the cuneiform guide 450 with the medial cuneiform 202.

As shown in FIGS. 28-30, the medial portion 456 includes a curvature which includes a radiused or otherwise arcuate portion extending between a first plane of the lower surface 454 that is substantially parallel with a portion of the surface of the medial cuneiform 202 and a second plane of the lower surface 454 that is substantially perpendicular relative to the first plane. In the previous example, the radiused or arcuate portion of the lower surface 454 may be contoured to a curvature of the medial cuneiform 202 of the patient. As shown, the lateral portion 458 includes a similarly curved geometry in order provide structural support for the contouring of the cuneiform guide 450. This feature may generally take the form of a “hook” shape or other similar geometry and extend around the top portion of the medial cuneiform 202 to contact an anatomical feature found on the lateral portion of the anatomy of the medial cuneiform 202. The medial portion 456 may include a similar feature to that of the lateral portion 458 which, in conjunction with the lower surface 454, provides structural support for a contouring feature of the lower surface 454 that is configured to contact one or more portions of the medial anatomy of the first metatarsal 206. The structural features of the lower surface 454 in conjunction with the medial and/or lateral portions 456, 458 of the cuneiform guide 450 may be configured to facilitate placement of the metatarsal guide 410 in a desired position by referencing known anatomical features of the topology of the medial cuneiform 202.

The medial and lateral portions 456, 458 of the cuneiform guide 450 each include an engagement feature 478, 474 which, as shown, includes a substantially circular feature extending from and positioned above (e.g., superior relative to) the respective medial and lateral portions 456, 458. The engagement features 478, 474 are each shown to include a texture 480, 476, shown in an exemplary embodiment as, for example, a plurality of ridges, which may be gripped or otherwise held by a physician when placing the cuneiform guide 450 in a desired position on the medial cuneiform 202 (or when performing other operations involving the cuneiform guide 450). In some aspects, the engagement features 478, 474 may have an alternate position about the cuneiform guide 450, for example extending from and/or above the lateral portion 458.

A shaft 460 is shown to extend upward from a central portion of the body 452 disposed substantially between the medial and lateral portions 456, 458. In some aspects, at least a portion of the shaft 460 includes a footprint that extends beyond that of the body 452. As shown, the shaft 460 includes a substantially oval or elliptically shaped cross-sectional geometry and is centered about a central axis extending substantially perpendicular to at least a portion of the surface of the medial cuneiform 202. A texture 464 is disposed on at least a portion of the outer surface of the shaft 460, shown in an exemplary embodiment as, for example, a plurality of ridges, so as to facilitate gripping of the shaft 460 by a physician or other medical personnel. The lower portion of the shaft 460 (which abuts the lower surface 454) configured to contact or be positioned adjacent the surface of the medial cuneiform 202.

The system 100 further includes a wire guide 466 which is integral with the body 452 and configured to facilitate the releasable coupling of the cuneiform guide 450 with the medial cuneiform 202. As shown in FIGS. 25-30, the wire guide 466 may include one or more of the same/similar features or dimensions as the wire guide 126. The wire guide 466 is shown to include a substantially oval or elliptical prism-shaped geometry (and accordingly a similarly shaped cross-sectional geometry). The wire guide 466 is further shown to include at least one bore (which, as shown in at least FIGS. 25-30 includes a pair of bores and will be referred to hereinafter as such) 468 extending from a top surface of the wire guide 466 through a body portion thereof to a bottom surface of the wire guide 466. The bores 468 are centered about respective central axes and have a substantially cylindrical geometry. Further, the bores 468 are configured such that the central axes thereof are positioned substantially parallel to one another (although in some aspects, the bores 468 and central axes thereof may be angled so as to achieve converging, diverging, or other non-parallel, oblique trajectories). As shown, the bores 468 are each configured to receive one of the stabilization wires 472 therein and therethrough and accordingly may be sized (e.g., diametrically, length-wise, etc.) to accommodate one or more sizes of the stabilization wires 472.

The cuneiform guide 450 may be implemented in conjunction with other components of the system 400 to couple the cuneiform guide 450 with the medial cuneiform 202 of the patient. The cuneiform guide 450 may first be positioned and placed by a physician such that at least a portion of the lower surface 454 interfaces with the surface of the medial cuneiform 202 of the patient. The cuneiform guide 450 may include one or more features, for example the bottom surface 454 and/or the medial and/or lateral portions 456, 458, that facilitate placement of the cuneiform guide 450 in a desired position (and may provide haptic feedback to a physician confirming proper placement/positioning of the guide). For example, the lower surface 454 and the medial portion 456 may collectively include a curvature configured to interface with a medial portion of the surface of the medial cuneiform 202. Further, the lower surface and/or the lateral portion 458 of the cuneiform guide 450 may include an extension or other feature configured to wrap around a portion of the medial cuneiform 202 so as to interface with and reference an anatomical feature of the medial cuneiform 202. For example, as shown in FIG. 26, the body 452 is shown to extend around a curvature of the distal portion of the medial cuneiform such that at least a portion of the body 452 may be positioned within the joint space 204 when the cuneiform guide 450 is placed in a desired position.

In placing the cuneiform guide 450 in a desired position, a physician may grip the cuneiform guide 450 via the texture 464 provided thereon, and/or may also grip the wire guide 466 by the engagement features 478, 474. Various features of the cuneiform guide 450, for example those of the lower surface 454 in conjunction with the medial and lateral portions 456, 458 as described previously herein may provide haptic feedback to a physician in that the one or more features retain the cuneiform guide 450 in a desired position when the feature(s) contact the corresponding complementary anatomical features to which they are contoured and referenced. Once the cuneiform guide 450 is placed in a desired position, one of the stabilization wires 472 may be placed at least partially within one of the bores 468 such that the pointed tip of the stabilization wire 472 is positioned adjacent the surface of the medial cuneiform 202. A physician may then couple the stabilization wire 472 with the medial cuneiform 202 using one or more instruments common to orthopedic surgical procedures, for example, a drill or other powered driver. In coupling the stabilization wire 472 with the medial cuneiform 202, the cuneiform guide 450 is also releasably coupled with the first metatarsal 206. Once a first stabilization wire 472 has been placed, a second stabilization wire 472 may also be placed according to the same procedure. Following placement of the pair of stabilization wires 472, the cuneiform guide 450 may be decoupled from the medial cuneiform 202 with the stabilization wires 472 remaining coupled with the medial cuneiform 202.

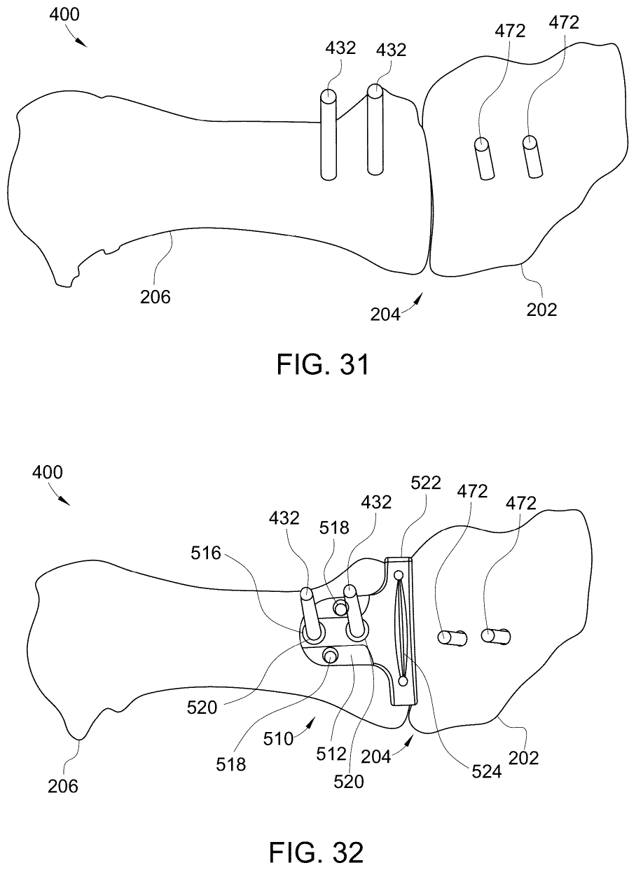

Referring now to FIGS. 31-39, a cut guide 510 is shown in conjunction with various components of the system 400 including, for example the stabilization wires 432, 472. The cut guide 510 may be implemented in conjunction with the system 400, or may also be implemented in conjunction with the systems 100, 300 as shown and described previously herein, or may also be implemented in conjunction with other alternative systems. The cut guide 510 is shown to be couplable with the stabilization wires 432, 472 of the system 400 (and thus indirectly coupled with the first metatarsal 206 and the medial cuneiform 202), but may also similarly couple with the same/similar anatomy when implemented in conjunction with other systems shown and described herein. In some aspects, the cut guide 510 may be reversible (e.g., can be used with both the first metatarsal 206 and the medial cuneiform 202) and/or may be universal (reversible with respect to various bones of the anatomy).

The cut guide 510 includes a top surface 512 opposite disposed opposite a body 516 from a bottom surface 514. As shown the top and bottom surfaces 512, 514 may be substantially parallel to one another over portions of the body 516 and/or may also include one or more radii or curvatures configured to facilitate placement on a similarly curved surface of the first metatarsal 206 and/or the medial cuneiform 202. The cut guide 510 is further shown to include a pair of openings 518 (“openings 518”) positioned opposite one another on obliquely angled portions of the top surface 512. Each of the openings 518 extend through the body 516 from the top surface 512 to the bottom surface 514 and comprise a central axis which is substantially perpendicular to the aforementioned obliquely-angled portion of the top surface 512. Accordingly, additional stabilization wires, the same as/similar to the stabilization wires 432, may be placed therein and therethrough the openings 518 so as to facilitate additional stabilization and coupling with the first metatarsal 206 and the medial cuneiform 202. The cut guide 510 is also shown to include a pair of openings 520 disposed substantially between the openings 518 and extending through the body 516 from the top surface 512 to the bottom surface 514. The openings 520 each include a central axis about which the openings 520 are positioned, with the central axes running substantially parallel to one another. Accordingly, the openings 520 are sized to receive at least a portion of the stabilization wires 432, 472 therein and therethrough, thus facilitating insertion of the cut guide 510 over the stabilization wires 432, 472 which have already been placed in the first metatarsal 206 and the medial cuneiform 202. One or both of the openings 518, 520 may include ramped or tapered features facilitating the insertion of stabilization wires therein.

The cut guide 510 includes an elongated portion 522 integral with and extending from the body 516, with the elongated portion 522 having a greater lateral dimension than that of the body 516. The elongated portion 522 is shown to include a slot 524 extending along a portion thereof, with the terminal ends of the slot 524 positioned adjacent the lateral edges of the elongated portion 522. The slot 524 is shown to extend from the top surface 512 through to the bottom surface 514 of the cut guide 510 and, in some aspects, may include a tapered portion adjacent the slot 514 so as to facilitate placement of a cutting blade therein. In some aspects, the slot 524 may include semi-cylindrical volumes/holes at opposite ends thereof in fluid communication with central portions of the slot 524.

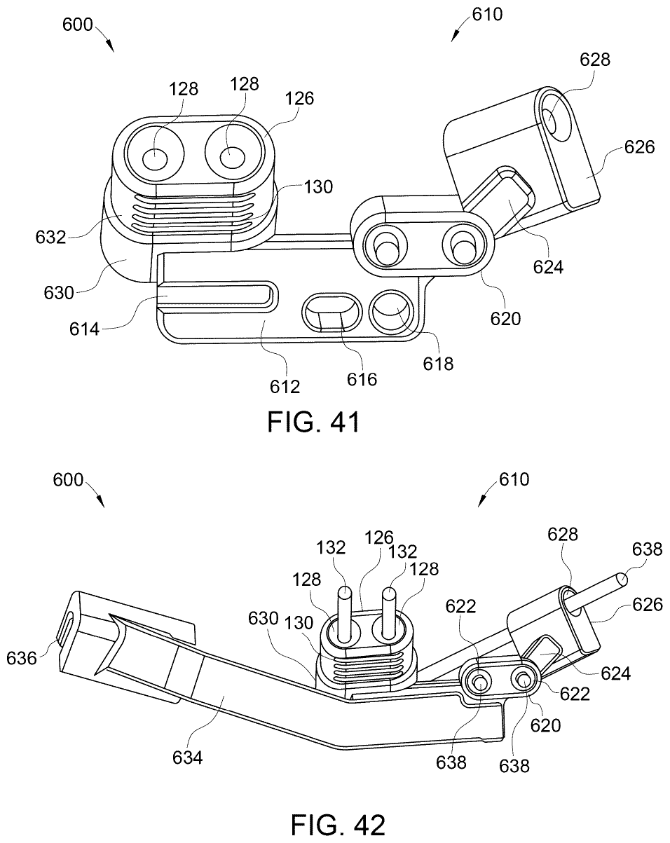

Referring to FIGS. 40-42, a system 600 for implementation in performing a surgical procedure of the Lapidus joint is shown, according to an exemplary embodiment. The system 600 may be implemented in conjunction with one or more of the systems 100, 300, and 400 shown and described herein and/or may be implemented in conjunction with additional systems. In some aspects, the system 600 may incorporate one or more components placed in anatomy in implementing previous systems, for example stabilization wires 132, 172 and/or the wire guide 126. In some aspects, the system 600 may be implemented in order to provide correction to a deformity of the Lapidus joint after one or more cuts have been made to the first metatarsal 206 and/or the medial cuneiform 202. In some aspects, one or more components of the system 600 may be specific to a patient so as to provide quantifiable correction from a measured deformity of the Lapidus joint corrected by one or more cuts to the first metatarsal 206 and/or the medial cuneiform 202 prior to implementation of the system 600.

The system 600 includes a guide 610, with the guide 610 having a body 612 including a slot 614. As shown, the slot 614 extends into the body 612 from a lateral edge thereof and has a substantially rectangular geometry. In some aspects, the slot 614 may include one or more grooves therein (e.g., a dovetail, etc.) to facilitate releasable coupling with other components of the system 600. The guide 610 also includes openings 616 and 618 extending through the body 612 from a top surface to the bottom surface and positioned adjacent the slot 614. The openings 616, 618 are configured to facilitate releasable coupling with various components with which the system 600 may be implemented. The guide 610 is further shown to include a shaft 620 extending laterally therefrom, with at least a portion of the shaft 620 extending beyond that of the body 612. The shaft 620 is shown to include a pair of openings 622 which are configured to receive a pair of stabilization wires, for example the stabilization wires 172, therein and therethrough and are accordingly parallel to one another. The shaft 620 also includes an arm 624 extending laterally therefrom and integral with a shaft 626. The shaft 626 includes an opening 628 which is configured to receive a stabilization wire 638, with the central axis (and corresponding trajectory) of the opening 628 oblique relative to and in another plane from the openings 622 of the shaft 620.

The guide 610 further includes a shaft 630 extending from an opposite end of the body 612 from the shaft 620, with the shaft having a substantially oval/elliptical geometry and defining a cavity 632 therein with a similarly shaped cross-sectional geometry. The wire guide 126 is receivable within the cavity 632 (which may be the same as and/or similar to the cavity 122 of the shaft 120) and is thus couplable with the guide 610 via the shaft 630 (in the same or a similar manner to that relative to the shaft 120). The stabilization wires 132 may then be manipulated within the bores 128 of the wire guide 126 so as to releasably couple the guide 610 with the first metatarsal 206.

Referring now to FIG. 42, the system 600 is further shown to include a guide arm 634 that is releasably couplable with the guide 610 via the slot 614. The guide arm includes an opening 636 configured to guide a trajectory of a stabilization wire or other similar device, with the trajectory being spaced from that of the stabilization wires 132, 172, and 638.

The system 600 and components thereof may be implemented after resectioning cuts have been made to the first metatarsal 206 and the medial cuneiform 202 of a patient and the cut guide 510 has been decoupled from the anatomy. The guide 610 may be manipulated such that the stabilization wires 172 are positioned within the openings 622 and, similarly, the stabilization wires 132 are positioned within the cavity 632 (and further, within the openings 128 of the wire guide 126 if the wire guide is inserted within the cavity 632). This may be performed with the guide 610 offset a distance from the first metatarsal 206 and the medial cuneiform 202 and, once the desired placement of the stabilization wires 132, 172 has been achieved, the guide 610 is advanced down the length the stabilization wires 132, 172 such that the guide 610 contacts or is positioned adjacent the first metatarsal 206 and the medial cuneiform 202. The manipulation of the stabilization wires 132, 172 subsequently biases the first metatarsal 206 and the medial cuneiform 202 from a first, uncorrected position to a second, corrected position. At the preference of the physician, the stabilization wire 638 may then be placed through the opening 628 and into the medial cuneiform 202 and subsequently the first metatarsal 206 in order to retain the correction achieved by the placement of the guide 610. In some aspects, the guide 610 may be removable from the stabilization wires 132, 172 (e.g., via a decoupling feature, removal of the stabilization wires 132, 172, or cut free). The arm 634 may then be coupled with the guide 610, with a stabilization wire or other component then placed along the trajectory guided by the opening 636 and into the first metatarsal 206 and, subsequently, the medial cuneiform 202 so as to retain correction of the Lapidus joint achieved by placement of the guide 610.

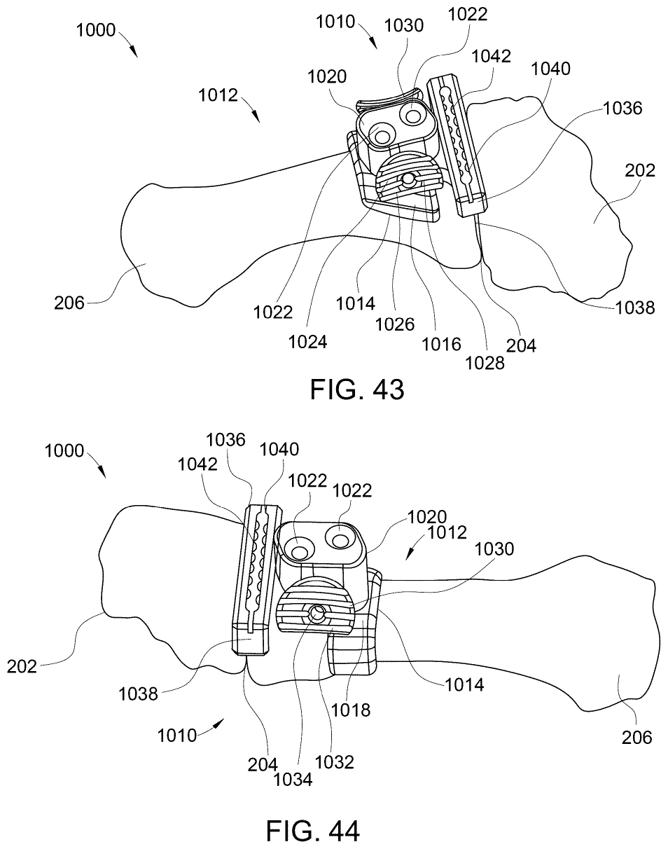

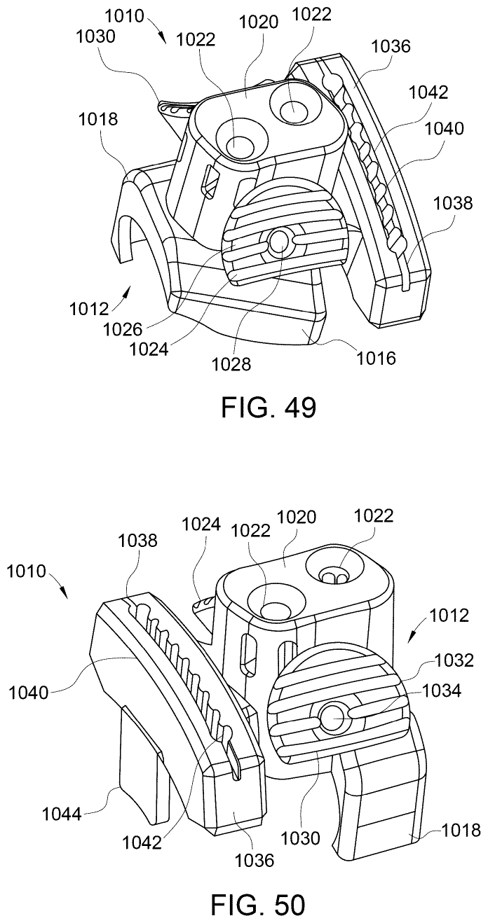

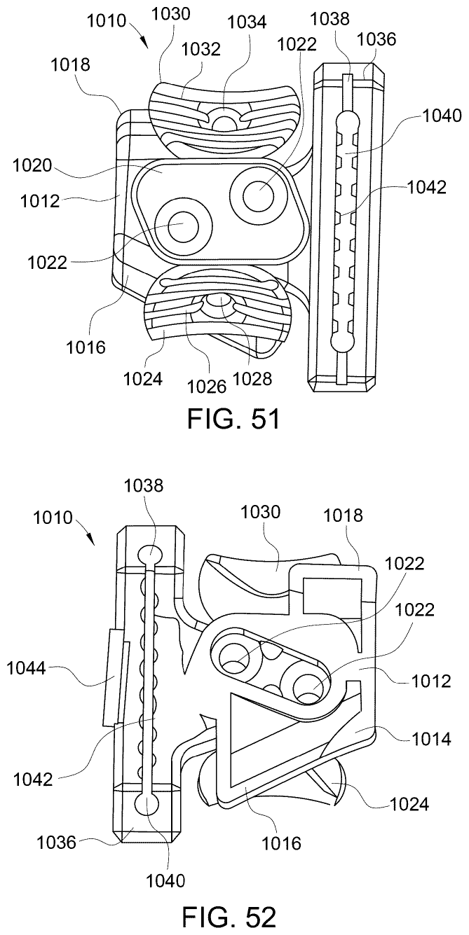

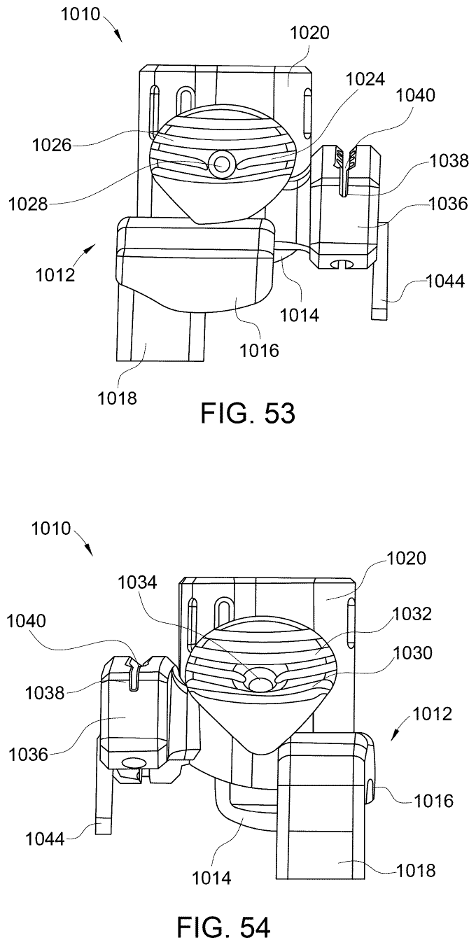

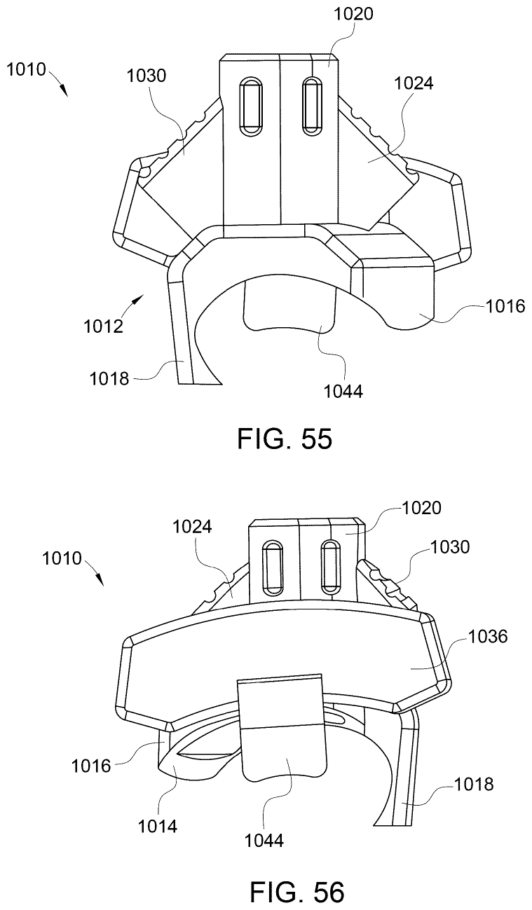

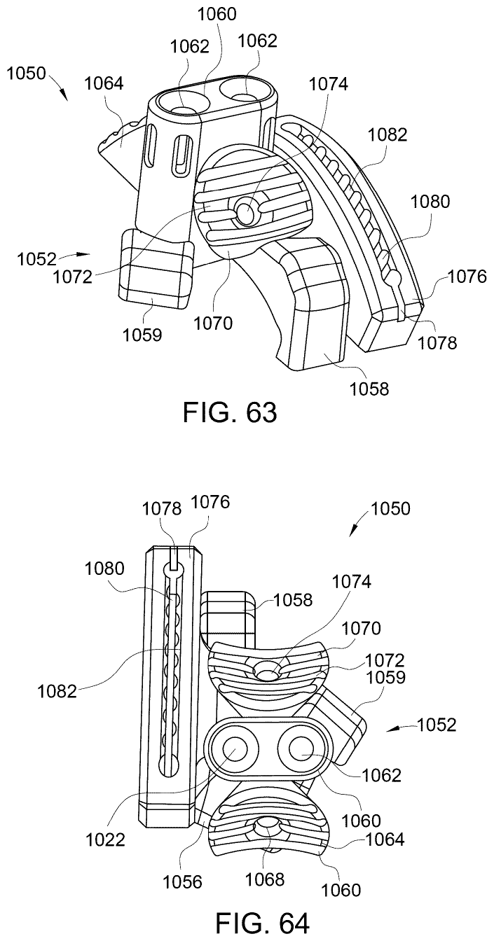

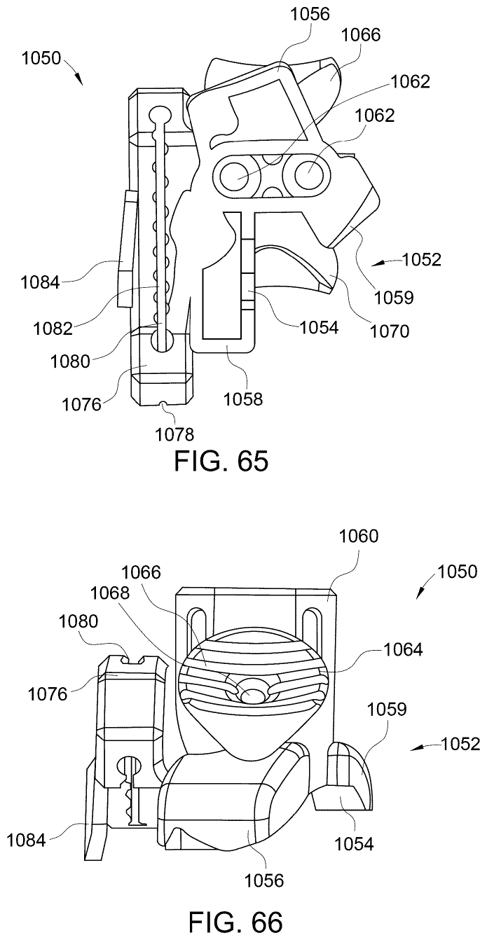

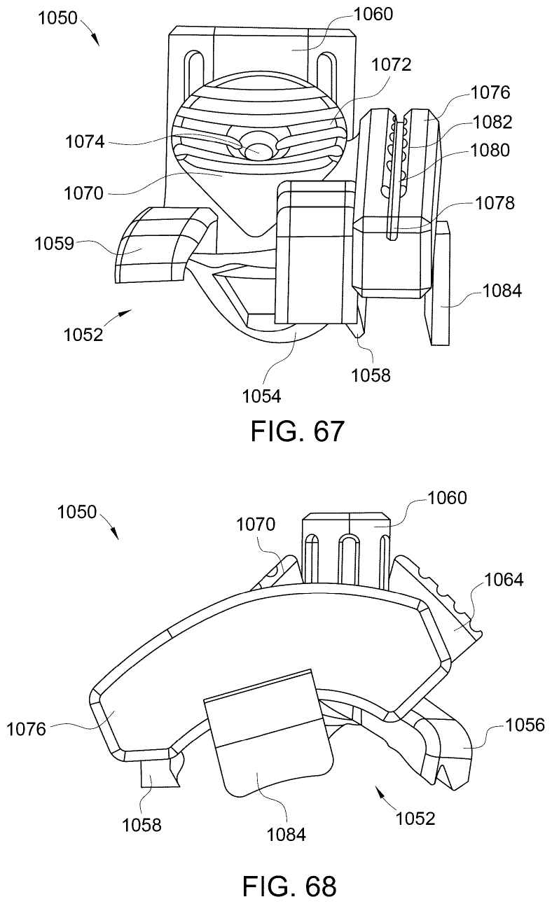

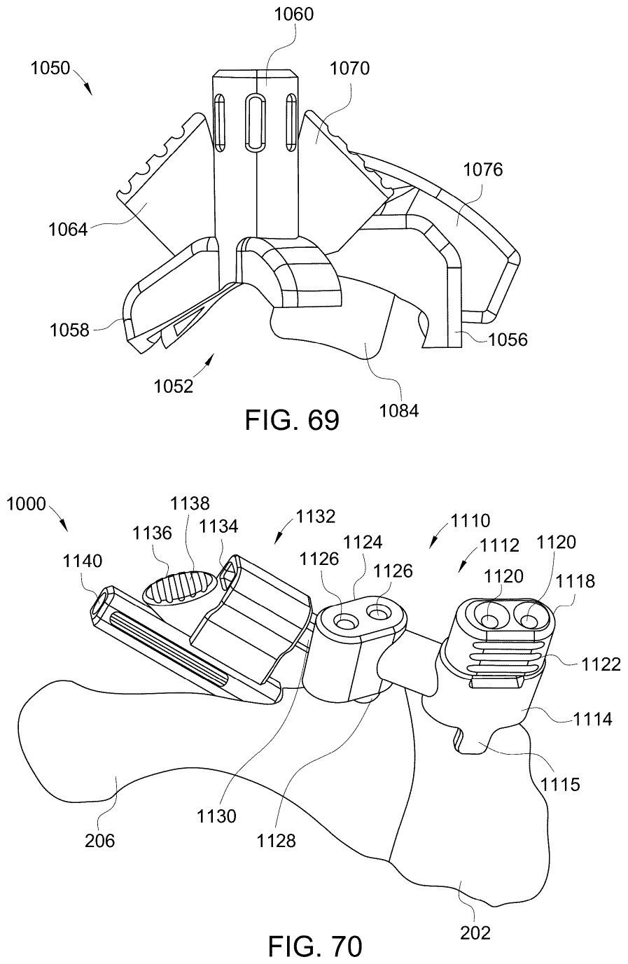

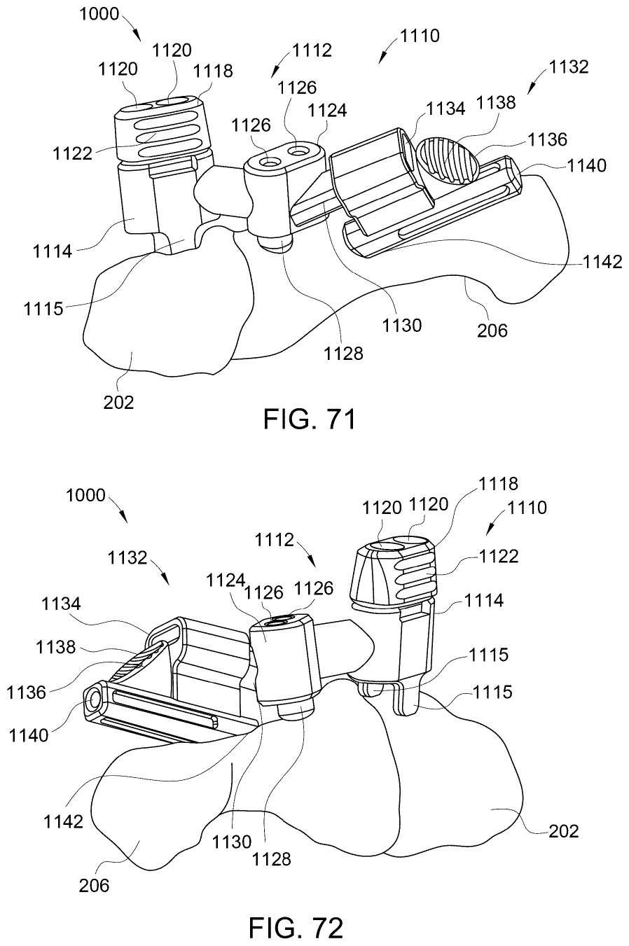

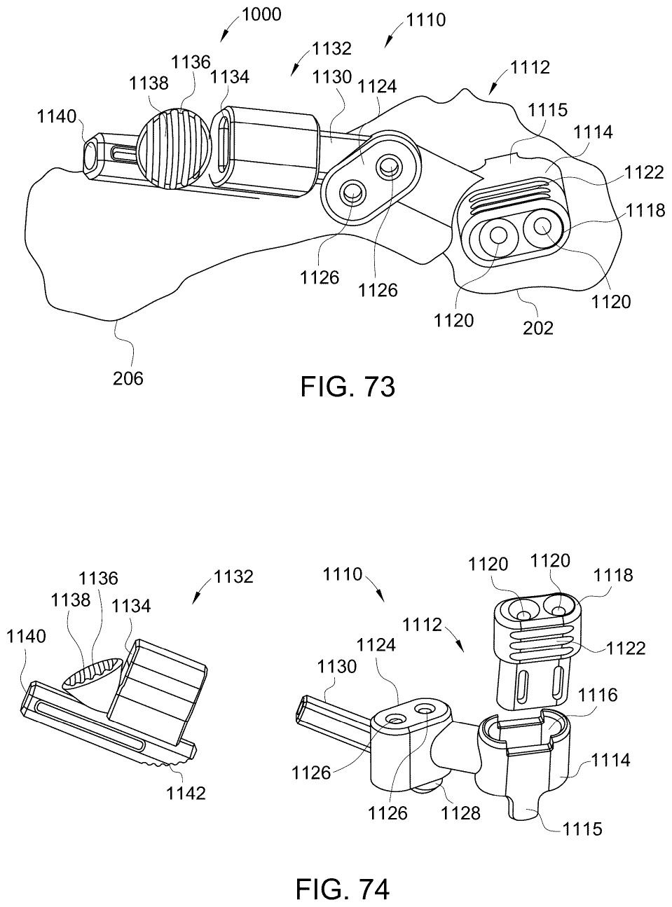

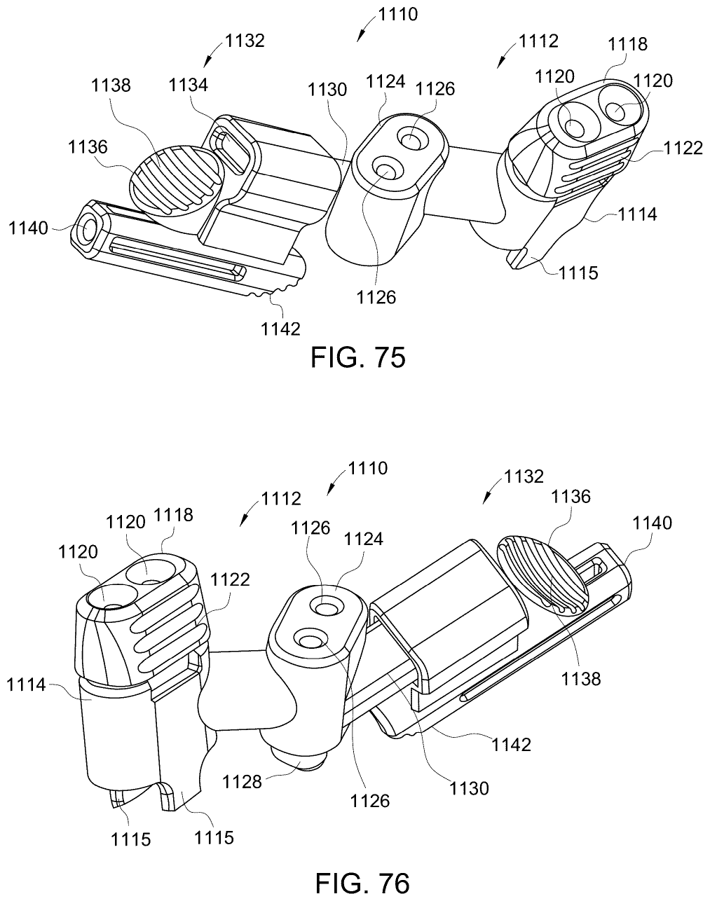

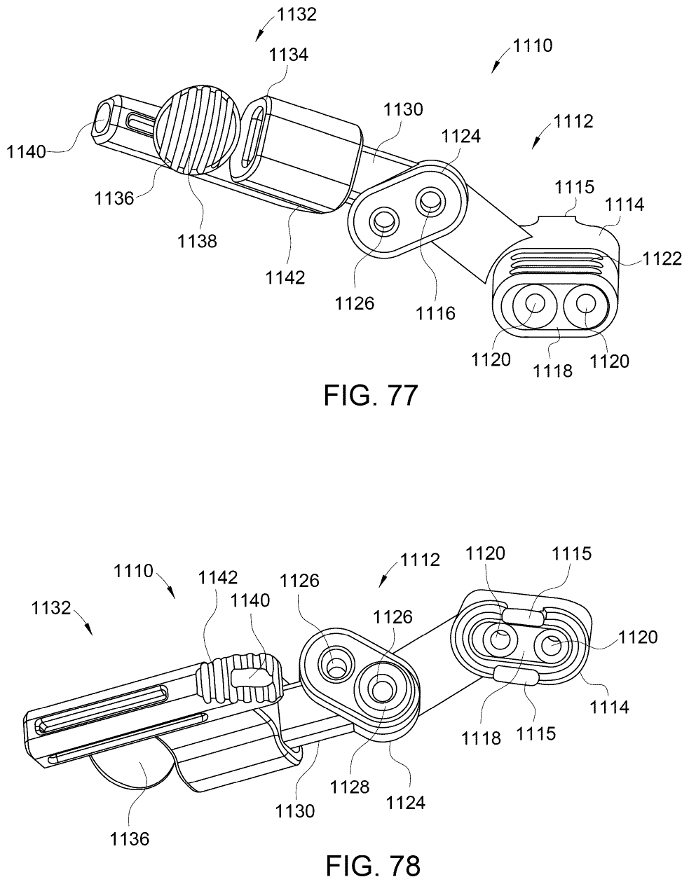

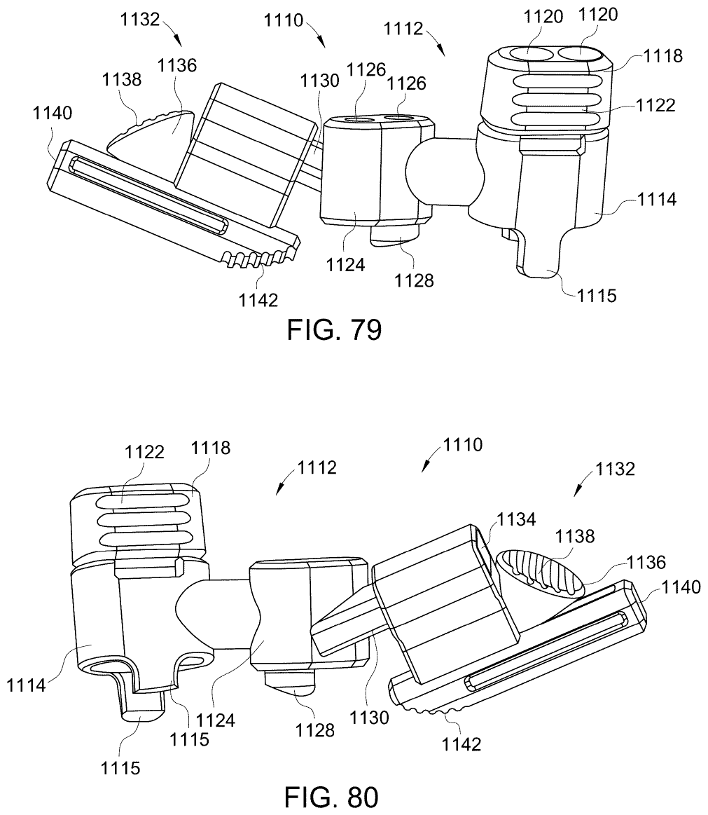

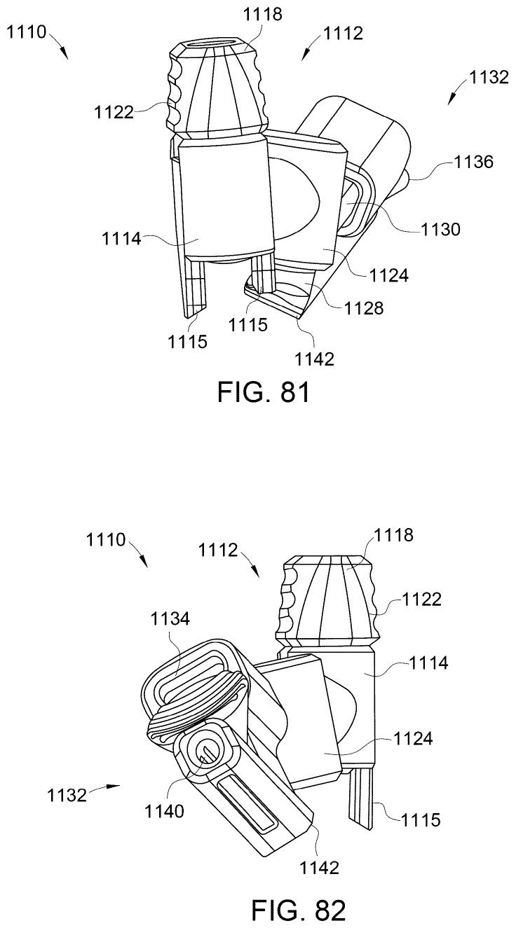

Referring now to FIGS. 43-82, a system 1000 and various components thereof are shown adjacent, coupled, and/or engaged with the medial cuneiform 202, the joint space 204, and/or the first metatarsal 206, according to an exemplary embodiment. In some aspects, one or more components of the system 1000 may be compatible with one or more components and/or systems including those shown and described previously herein. Similarly, the system 1000 and components thereof may also be compatible with one or more implant systems configured to provide fixation and/or facilitate fusion across the joint between the medial cuneiform 202 and the first metatarsal 206 (e.g., once the aforementioned bones have been prepared and positioned for fusion, thus eliminating the joint space 204). In some aspects, the system 1000 may include one or more components in addition to those shown and described herein. For example, one or more components of the system 1000 may include those common to operating rooms and/or orthopedic procedures, for example stabilization wires (e.g., “k-wires”), powered drivers and other powered instruments, sagittal and/or reciprocating saws and corresponding blades, powered burrs, and other common surgical instruments.

With reference to FIGS. 43-56, a patient-specific guide 1010 (referred to hereinafter as “guide 1010”) is shown, according to an exemplary embodiment. The guide 1010 is shown to include a body portion 1012 (referred to hereinafter as “body 1012”) positioned in a central portion thereof. The guide 1010 may include one or more components that are configured specifically based on the anatomy of the patient. For example, at least a portion of a lower surface 1014 of the body 1012 of the guide 1010 is configured to match at least a portion of the outer surface of the first metatarsal 206 (e.g., at least a portion of the lower surface 1014 is surface matching to the first metatarsal 206). Accordingly, the guide 1010 may be placed on the first metatarsal 206 and manipulated such that surface matching portions of the lower surface 1014 contact or are positioned adjacent to the corresponding portions of the first metatarsal 206 to which the portions of the lower surface 1014 are matched. Therefore, the lower surface 1014 may include one or more curvatures, convexities, concavities, or other geometric features to accommodate topology of the anatomy of a given patient.

The guide 1010 is shown to include a medial portion 1016 positioned substantially opposite the body 1012 from a lateral portion 1018, as shown in at least FIGS. 43-48 (where the lateral and medial directions are with respect to the patient anatomy). The medial portion 1016 may be configured to extend laterally (e.g., sideways, in a medial direction) from the body 1012 of the guide 1010. In some aspects, the medial portion 1016 may include one or more radii and/or curvatures (which may be the same as, similar to, and/or in conjunction with the curvature of the lower surface 1014). When positioned on the first metatarsal 206 of the patient, at least a portion of the medial portion 1016 may extend from the top surface of the first metatarsal 206 or a portion adjacent thereto around to a medial side of the first metatarsal 206. In some aspects, the medial portion 1016 may include one or more extensions, cornered and/or angled portions, or other features in order to optimize contact and/or engagement between the medial portion 1016 and the medial aspects of the first metatarsal of the patient. Further, in some aspects, the medial portion 1016 may include one or more concavities and/or convexities on one or more surfaces thereof configured to contact and/or engage with a portion of the surface of the first metatarsal 206 and thus facilitate positional retention of the guide 1010 relative to the first metatarsal 206.

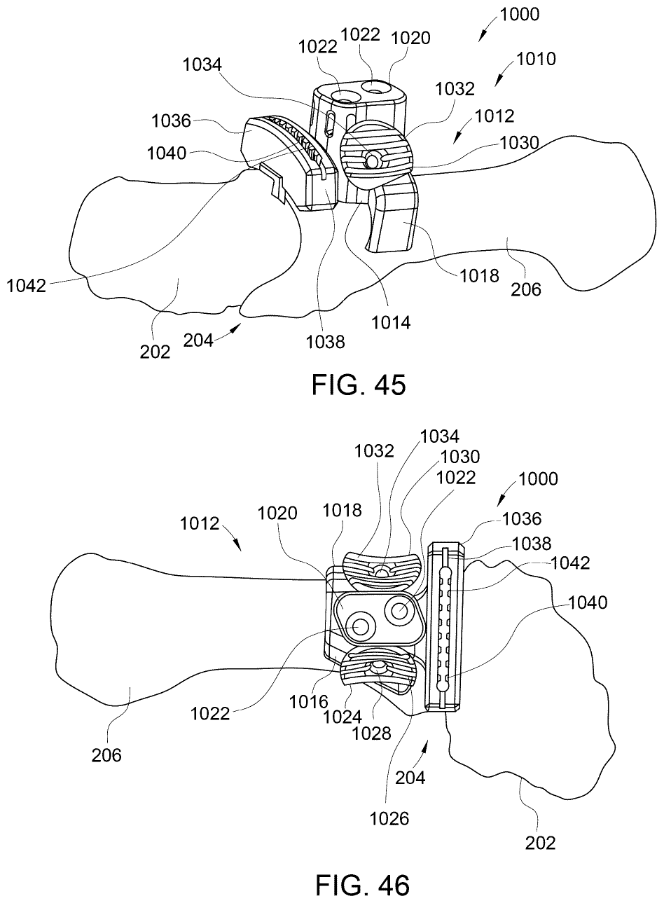

The lateral portion 1018 is positioned substantially opposite the body 1012 from the medial portion 1016 as shown in at least FIG. 45. The lateral portion 1018 is shown to extend laterally (e.g., sideways, and in the lateral direction relative to the anatomy of the patient) from the body 1018 and contact a portion of the first metatarsal 206 more inferior/plantar relative to other elements of the first metatarsal that are contacted by the guide 1010. In some aspects, the lateral portion 1018 may extend from a central portion or one of the end portions of the body 1012, or may extend continuously along the lateral side of the body 1012. Similar to the medial portion 1016, the lateral portion 1018 may include one or more radii, curvatures, or other geometric features configured to facilitate engagement and positional retention of the guide 1010 relative to the first metatarsal 206. For example, the lateral portion 1018 is shown in FIG. 45 to be a substantially rectangularly-shaped projection extending laterally (sideways) from the body 1012, and also extending downward from the body 1012 to contact lateral aspects of the first metatarsal 206. In some aspects, the size of the lateral projection 1018 may vary according to patient anatomy (e.g., position and size of the first metatarsal 206 of the patient). Further, in some aspects, the lateral portion 1018 may include one or more concavities and/or convexities on one or more surfaces thereof configured to contact and/or engage with a portion of the surface of the first metatarsal 206 and thus facilitate positional retention of the guide 1010 relative to the first metatarsal 206.

The body 1012 of the guide 1010 is shown to include a shaft 1020 positioned centrally on the body 1012 and extending substantially upward therefrom. The shaft 1020 is shown to be integral with the body 1012, although is some aspects the shaft 1020 (and/or similar components or components thereof) may be releasably couplable with the body 1012 and/or other components of the guide 1010. As shown in at least FIG. 43, the shaft 1020 extends upward from the body 1012 in a direction substantially perpendicular to a point on the surface of the first metatarsal 206 that is positioned within the footprint of the shaft 1020 when configured as shown in at least FIG. 43. In some aspects the shaft 1020 may also or alternatively extend upward in a direction substantially perpendicular to at least a portion of the lower surface 1014 of the guide 1010. As shown in FIGS. 43-56, the shaft 1020 has a substantially polygonal cross-section (e.g., a trapezoid or diamond shape as shown) and further has a substantially polygonal prism-shaped geometry. As shown, the shaft 1020 has substantially rounded edges/corners thereof. In some aspects, the shaft 1020 may also have other various geometries and cross-sections.

The shaft 1020 is shown to include a pair of openings 1022 extending vertically through the shaft 1020 from a top surface of the shaft 1020 to a bottom surface of the shaft 1020 (where the bottom surface of the shaft 1020 may be the same as, adjacent to, and/or overlapping with at least a portion of the lower surface 1014 of the guide 1010). As shown, the openings 1022 are positioned adjacent opposing corners/edges of the geometry of the shaft 1022, but may be positioned in other orientations in some aspects. Further, the openings 1022 are shown to have a substantially cylindrical geometry (e.g., a bore) and are further configured to be parallel (e.g., central axes thereof are parallel to one another). However, in some aspects the openings 1022 may have axes that are substantially oblique relative to one another. The openings 1022 are also configured to receive a k-wire or other surgical instrument/fixation component at least partially therein and therethrough, for example to facilitate releasable coupling between the guide 1010 and the first metatarsal 206.

The guide 1010 includes a medial engagement feature 1024 and a lateral engagement feature 1030 which are positioned on medial and lateral sides of the guide 1010, respectively (e.g., the medial engagement feature 1024 is positioned adjacent the medial portion 1016 and the lateral engagement feature 1030 is positioned adjacent the lateral portion 1018). The medial and lateral engagement features 1024, 1030 (or at least a portion thereof), are shown to be integral with and extend from the body 1012 in both upward and lateral directions (medial and lateral directions, respectively). In some aspects, at least a portion of the medial and lateral engagement features 1024, 1030 may extend from the body 1012 at approximately a 45-degree angle relative to a plane parallel with the top surface of the shaft 1020 (and/or from a top surface of the first metatarsal 206 when the guide 1010 is engaged with the first metatarsal 206 as shown in FIG. 45, and/or relative to at least a portion of the lower surface 1014 of the guide 1010). In some aspects, at least a portion of the medial and lateral engagement features 1024, 1030 may be tapered. For example, as shown in FIGS. 43-56, the medial and lateral engagement features 1024, 1030 each have a lesser volume and cross-sectional area adjacent the body 1012 than adjacent the terminal surfaces thereof. In some aspects the lower surface 1014 may include one or more depressions/recessions thereon and/or extending therein so as to accommodate soft tissue/periosteum adjacent one or more bones to recess into the features to allow for better grip/positioning of the guide 1010.

The medial and lateral engagement features 1024, 1030 are shown to have substantially circular and/or semi-circular geometries, as shown in the exemplary embodiment of FIGS. 43-56. In some aspects, the medial and lateral engagement features 1024, 1030 may include a semi-circular cross-sectional portion, for example that of a partial circle or oval also include at least one straight edge connecting opposite points of an outer curve/circumference. The medial and lateral engagement features 1024, 1030 are shown to include a depression (e.g., a concavity) positioned on at least a portion of the surface thereof so as to facilitate ergonomic gripping and manipulation of the guide 1010 by a physician with a fingertip in contact with the medial and lateral engagement features 1024, 1030. In some aspects, the concavity of the medial and lateral engagement features 1024, 1030 may include a texture 1032 as shown in at least FIGS. 43-44 to facilitate gripping of the medial and lateral engagement features 1024, 1030 by a user (e.g., a physician). The medial and lateral engagement features 1024, 1030 may be positioned such that application of a force to one or both of the medial and lateral engagement features 1024, 1030 is configured to bias the guide 1010 into a desired position relative to the first metatarsal 206.

The medial and lateral engagement features 1024, 1030 are each shown to include an opening 1028, 1034, extending from a surface thereof (e.g., the surface with the textures 1026, 1032) through the medial and lateral engagement features 1024, 1032 and terminating at or adjacent to the lower surface 1014 of the guide 1010. As shown, the openings 1028, 1034 have substantially cylindrical geometries and include axes positioned at a substantially oblique angle relative to one another. In some aspects, one or both of the openings 1028, 1034 may be configured to receive a k-wire or other similar stabilization element (e.g., an olive wire) at least partially therethrough to as to facilitate coupling of the guide 1010 with the first metatarsal 206. It should be understood that the trajectories of the openings 1028, 1034 (and any wires placed therethrough) do not intersect with either of the trajectories of the openings 1022 of the shaft 1020.

The guide 1010 is further shown to include a cut guide 1036 which, when viewed from a medial perspective, is positioned proximal relative to the shaft 1020 of the guide 1010. As shown, the cut guide 1036 is integral with the body 1012 and extends at least partially laterally (e.g., to the side) therefrom. The cut guide 1036 may be positioned relative to other components of the guide 1010 such that, when the guide 1010 is in a desired position on and engaged with the first metatarsal 206, the cut guide 1036 is positioned over a proximal-most portion of the first metatarsal 206 adjacent to the first tarsometatarsal joint 204.

The cut guide 1036 is shown to include a notch 1038 positioned adjacent slot 1040, as shown in at least FIG. 44. The slot 1040 is configured to receive at least a portion of a cutting instrument (e.g., a saw, which may be coupled with a power source or powered driver) at least partially therein/therethrough so as to facilitate guided resection of the first metatarsal 206 positioned beneath the slot 1040. The slot 1040 is shown to extend from a point adjacent the medial side/edge to a point adjacent the lateral side/edge of the cut guide 1036 and from a top surface through to the bottom surface of the cut guide 1036. Accordingly, the slot 1040 is configured to provide fluid communication between top and bottom surfaces of the cut guide 1036 (as the slot 1040 extends vertically therethrough) but does not provide fluid communication between medial and lateral surfaces of the cut guide 1036. In some aspects, the slot 1040 may be positioned in a plane that is parallel or substantially parallel to one or more of the axes of the openings 1022, or may be in a plane that is oblique relative to one or more of the axes of the openings 1022. As shown, the slot 1040 is a substantially elongated, rectangular geometry. In some aspects, the slot 1040 may include one or more partial geometries along the length thereof. For example, as shown in at least FIG. 44, the slot 1040 includes circular openings overlapping with the slot 1040 and extending through the cut guide 1036 from the top surface of the cut guide through to the bottom surface thereof.

The notch 1038 includes portions at opposing ends of the slot 1040 (e.g., opposite the longest dimension of the slot 1040), with an interruption in the notch 1038 to accommodate the slot 1040 and the opening thereof. The notch 1038 is shown to be substantially linear and has a depth that is a fraction of that of the slot 1040. The notch 1038 is configured such that a saw blade (or other similar thin, linear component) may be placed in a standing, lengthwise orientation at least partially within the notch 1038 such that the notch 1038 retains the saw blade in the standing, lengthwise position. In some aspects, a physician may position a sawblade within the notch 1038 while the guide 1010 is in a desired position on and coupled with the first metatarsal 206. Accordingly, in a procedure, a physician may then use fluoroscopic or other radiographic imaging techniques under which the saw blade within the notch 1038 will approximate a cut plane relative to the first metatarsal 206 along which the slot 1040 of the cut guide 1036 will guide a cut to the first metatarsal 206. Such a step may not be necessary or required in procedures, but may be performed by a physician in order to visualize the aforementioned cut plane in the first metatarsal 206 and/or to confirm placement of the guide 1010 relative to the first metatarsal 206 and the cut plane thereof.

The slot 1040 of the cut guide 1036 is shown to include one or more projections positioned on lateral sides adjacent to the slot 1040 and/or within the slot 1040. In some aspects, the projections may be configured to minimize movement of a saw blade when positioned within/through the slot 1040 when making cuts to bone (e.g., to minimize and/or dampen any vibration of the blade as a result of contact with the first metatarsal 206). Further, the projections may also be configured to minimize heat generation during use of the saw blade within the slot 1040 (e.g., heat generated as a result of friction against the sides of the cut guide 1036 defining the slot 1040.

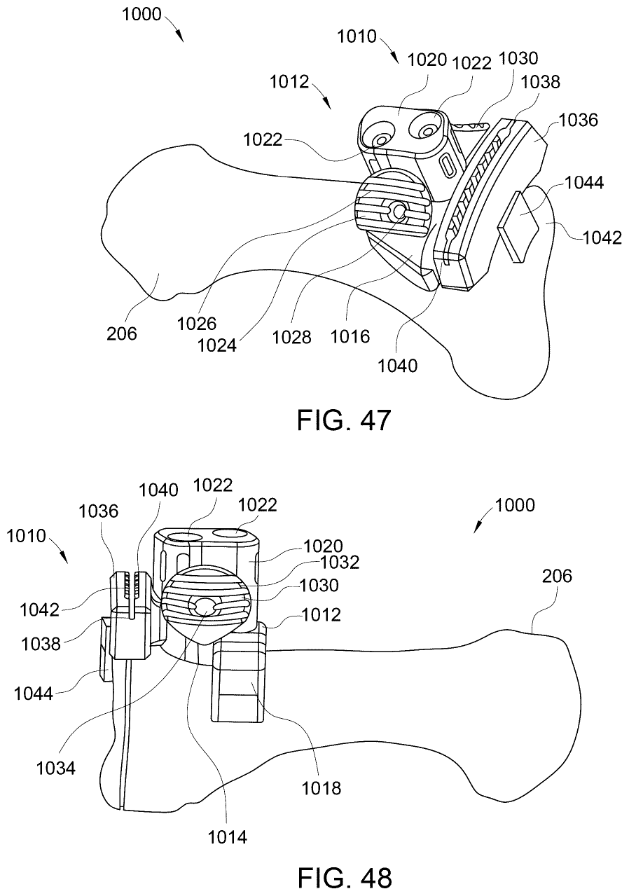

The cut guide 1036 is further shown to include a projection 1044 extending downward from a proximal side of the cut guide 1036 (where the proximal side of the cut guide 1036 is the side positioned most proximally relative to the anatomy of the patient as shown in at least FIG. 46. As shown, the projection 1044 has a lesser lateral dimension (e.g., in the medial-lateral direction) than the cut guide 1036, and extends downward below the bottom surface of the cut guide 1036. The projection 1044, as shown in at least FIGS. 47-48, is configured to extend into the first tarsometatarsal joint 204 adjacent to a proximal-most surface of the first metatarsal 206 when placed in a desired position relative to the first metatarsal 206. In some aspects, the projection 1044 may also be positioned adjacent to or abutting the medial cuneiform 202. Further, it should be understood that the vertical plane defined by the cut slot 1040 is positioned between the projection 1044 and the body 1012 of the guide 1010. In some embodiments, the projection 1044 may be variable relative to anatomy of the patient (e.g., will be lengthened or shortened, angled, etc. relative to anatomy of the first metatarsal 206 of the patient. Additionally, the projection 1044 may be configured as an indicator to the physician as to correct/incorrect positioning of the guide 1010 on the first metatarsal 206 of the patient. For example, the projection 1044 will contact the first metatarsal 206 and raise a portion of the guide 1010 when placed incorrectly and will be properly seated in the joint space allowing the lower surface 1014 of the guide 1010 to contact the first metatarsal 206 when the guide 1010 is positioned correctly.

Referring now to FIGS. 43-82, a system 1000 and various components thereof are shown adjacent, coupled, and/or engaged with the medial cuneiform 202, the joint space 204, and/or the first metatarsal 206, according to an exemplary embodiment. In some aspects, one or more components of the system 1000 may be compatible with one or more components and/or systems including those shown and described previously herein. Similarly, the system 1000 and components thereof may also be compatible with one or more implant systems configured to provide fixation and/or facilitate fusion across the joint between the medial cuneiform 202 and the first metatarsal 206 (e.g., once the aforementioned bones have been prepared and positioned for fusion, thus eliminating the joint space 204). In some aspects, the system 1000 may include one or more components in addition to those shown and described herein. For example, one or more components of the system 1000 may include those common to operating rooms and/or orthopedic procedures, for example stabilization wires (e.g., “k-wires”), powered drivers and other powered instruments, sagittal and/or reciprocating saws and corresponding blades, and other common surgical instruments.

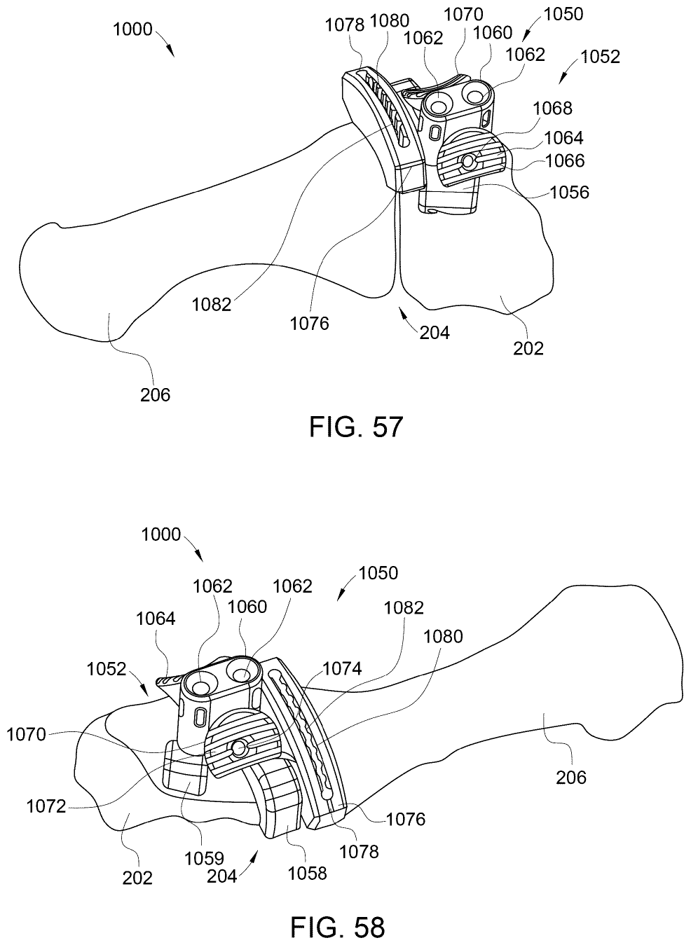

With reference to FIGS. 57-69, a patient-specific guide 1050 (referred to hereinafter as “guide 1050”) is shown, according to an exemplary embodiment. The guide 1050 includes a body portion 1052 (referred to hereinafter as “body 1052”) positioned in a central portion thereof. The guide 1050 may include one or more components that are configured specifically based on the anatomy of the patient. For example, at least a portion of a lower surface 1054 of the body 1052 of the guide 1050 is configured to match at least a portion of the outer surface of the medial cuneiform 202 (e.g., at least a portion of the lower surface 1054 is surface matching to the medial cuneiform 202). Accordingly, the guide 1050 may be placed on the medial cuneiform 202 and manipulated such that surface matching portions of the lower surface 1054 contact or are positioned adjacent to the corresponding portions of the medial cuneiform 202 to which the portions of the lower surface 1054 are matched. Therefore, the lower surface 1054 may include one or more curvatures, convexities, concavities, or other geometric features to accommodate topology of the anatomy of a given patient.

The guide 1050 is shown to include a medial portion 1056 positioned substantially opposite the body 1052 from a lateral portion 1058, as shown in at least FIGS. 58 and 60 (where the lateral and medial directions are with respect to the patient anatomy). The medial portion 1056 may be configured to extend laterally (e.g., sideways, in a medial direction) from the body 1052 of the guide 1050. In some aspects, the medial portion 1056 may include one or more radii and/or curvatures (which may be the same as, similar to, and/or in conjunction with the curvature of the lower surface 1054). When positioned on the medial cuneiform 202 of the patient, at least a portion of the medial portion 1056 may extend from the top surface of the medial cuneiform 202 or a portion adjacent thereto around to a medial side of the medial cuneiform 202. In some aspects, the medial portion 1056 may include one or more extensions, cornered and/or angled portions, or other features in order to optimize contact and/or engagement between the medial portion 1056 and the medial aspects of the medial cuneiform 202 of the patient. Further, in some aspects, the medial portion 1056 may include one or more concavities and/or convexities on one or more surfaces thereof configured to contact and/or engage with a portion of the surface of the medial cuneiform 202 and thus facilitate positional retention of the guide 1050 relative to the medial cuneiform 202.

The lateral portion 1058 is positioned substantially opposite the body 1052 from the medial portion 1056, as shown in at least FIG. 58. The lateral portion 1058 is shown to extend laterally (e.g., sideways, and in the lateral direction relative to the anatomy of the patient) from the body 1052. In some aspects, the lateral portion 1058 may extend from a central portion or one of the end portions of the body 1052, or may extend continuously along the lateral side of the body 1052. Similar to the medial portion 1056, the lateral portion 1058 may include one or more radii, curvatures, or other geometric features configured to facilitate engagement and positional retention of the guide 1050 relative to the medial cuneiform 202. For example, the lateral portion 1058 is shown in FIG. 58 to be a substantially rectangularly-shaped projection extending laterally (sideways) from the body 1052, and also extending downward from the body 1052 to contact lateral aspects of the medial cuneiform 202. In some aspects, the size of the lateral projection 1058 may vary according to patient anatomy (e.g., position and size of the medial cuneiform 202 of the patient). Further, in some aspects, the lateral portion 1058 may include one or more concavities and/or convexities on one or more surfaces thereof configured to contact and/or engage with a portion of the surface of the medial cuneiform 202 and thus facilitate positional retention of the guide 1050 relative to the medial cuneiform 202.

The guide 1050 further includes a proximal portion 1059 which, as shown in in at least FIG. 58, extends in a substantially proximal-lateral direction from the body 1052 (e.g., is positioned proximally between the medial portion 1056 and the lateral portion 1058). As shown the proximal portion 1059 forms an approximately 45-degree angle relative to the lateral portion 1058 (however, this angle may be adjusted to angles other than 45-degrees, for example from 0 to 90-degrees, based on anatomy of a patient). In some aspects, the proximal portion 1059 may be configured to limit rotation of the guide 1050 relative to one or more k-wires coupling the guide 1050 with the medial cuneiform 202. Similar to the lateral portion 1058, the proximal portion 1059 includes a substantially curved, rectangular geometry which may be contoured to be positioned adjacent and/or abut the medial cuneiform 202. In some aspects, the proximal portion 1059 may be positioned more proximally than shown in FIG. 58 (e.g., forming a greater angle between the proximal portion 1059 and the lateral portion 1058), or may be positioned more laterally than shown (e.g., forming a lesser angle between the proximal portion 1059 and the lateral portion 1058).

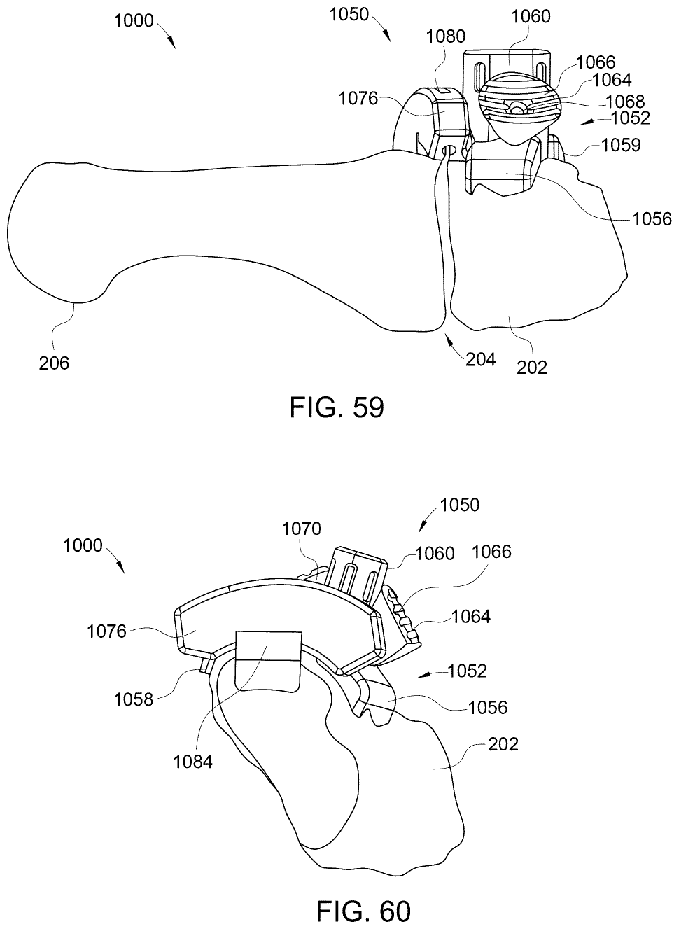

The body 1052 of the guide 1050 is shown to include a shaft 1060 positioned centrally on the body 1052 and extending substantially upward therefrom. The shaft 1060 is shown to be integral with the body 1052, although is some aspects the shaft 1060 (and/or similar components or components thereof) may be releasably couplable with the body 1052 and/or other components of the guide 1050. As shown in at least FIG. 59, the shaft 1060 extends upward from the body 1052 in a direction substantially perpendicular to a point on the surface of the medial cuneiform 202 that is positioned within the footprint of the shaft 1060 when configured as shown in at least FIG. 58. In some aspects the shaft 1060 may also or alternatively extend upward in a direction substantially perpendicular to at least a portion of the lower surface 1054 of the guide 1050. As shown in FIGS. 57-69, the shaft 1060 has a substantially polygonal cross-section (e.g., a trapezoid or diamond shape as shown) and further has a substantially polygonal prism-shaped geometry. As shown, the shaft 1060 has substantially rounded edges/corners thereof. In some aspects, the shaft 1060 may also have other various geometries and cross-sections.

The shaft 1060 is shown to include a pair of openings 1062 extending vertically through the shaft 1060 from a top surface of the shaft 1060 to a bottom surface of the shaft 1060 (where the bottom surface of the shaft 1060 may be the same as, adjacent to, and/or overlapping with at least a portion of the lower surface 1054 of the guide 1050). As shown, the openings 1062 are positioned adjacent opposing corners/edges of the geometry of the shaft 1062, but may be positioned in other orientations in some aspects. Further, the openings 1062 are shown to have a substantially cylindrical geometry (e.g., a bore) and are further configured to be parallel (e.g., central axes thereof are parallel to one another). The openings 1062 are also configured to receive a k-wire or other surgical instrument at least partially therein and therethrough, for example to facilitate releasable coupling between the guide 1050 and the medial cuneiform 202.

The guide 1050 includes a medial engagement feature 1064 and a lateral engagement feature 1070 which are positioned on medial and lateral sides of the guide 1050, respectively (e.g., the medial engagement feature 1064 is positioned adjacent the medial portion 1056 and the lateral engagement feature 1070 is positioned adjacent the lateral portion 1058). The medial and lateral engagement features 1064, 1070 (or at least a portion thereof), are shown to be integral with and extend from the body 1052 in both upward and lateral directions (medial and lateral directions, respectively). In some aspects, at least a portion of the medial and lateral engagement features 1064, 1070 may extend from the body 1052 at approximately a 45-degree angle relative to a plane parallel with the top surface of the shaft 1060 (and/or from a top surface of the medial cuneiform 202 when the guide 1050 is engaged with the medial cuneiform 202 as shown in FIG. 60, and/or relative to at least a portion of the lower surface 1054 of the guide 1050). In some aspects, at least a portion of the medial and lateral engagement features 1064, 1070 may be tapered. For example, as shown in FIGS. 57-69, the medial and lateral engagement features 1064, 1070 each have a lesser volume and cross-sectional area adjacent the body 1052 than adjacent the terminal surfaces thereof.

The medial and lateral engagement features 1064, 1070 are shown to have substantially circular and/or semi-circular geometries, as shown in the exemplary embodiment of FIGS. 57-69. In some aspects, the medial and lateral engagement features 1064, 1070 may include a semi-circular cross-sectional portion, for example that of a partial circle or oval also include at least one straight edge connecting opposite points of an outer curve/circumference. The medial and lateral engagement features 1064, 1070 are shown to include a depression (e.g., a concavity) positioned on at least a portion of the surface thereof so as to facilitate ergonomic gripping and manipulation of the guide 1050 by a physician with a fingertip in contact with the medial and lateral engagement features 1064, 1070. In some aspects, the concavity of the medial and lateral engagement features 1064, 1070 may include a texture 1072 as shown in at least FIGS. 57-58 to facilitate gripping of the medial and lateral engagement features 1064, 1070 by a user (e.g., a physician).

The medial and lateral engagement features 1064, 1070 are each shown to include an opening 1068, 1074, extending from a surface thereof (e.g., the surface with the textures 1066, 1072) through the medial and lateral engagement features 1064, 1070 and terminating at or adjacent to the lower surface 1054 of the guide 1050. As shown, the openings 1068, 1074 have substantially cylindrical geometries and include axes positioned at a substantially oblique angle relative to one another. In some aspects, one or both of the openings 1068, 1074 may be configured to receive a k-wire or other similar stabilization element (e.g., an olive wire) at least partially therethrough to as to facilitate coupling of the guide 1050 with the medial cuneiform 202. It should be understood that the trajectories of the openings 1068, 1074 (and any wires placed therethrough) do not intersect with either of the trajectories of the openings 1062 of the shaft 1060.

The guide 1050 is further shown to include a cut guide 1076 which, when viewed from a medial perspective, is positioned distal relative to the shaft 1060 of the guide 1050. As shown, the cut guide 1076 is integral with the body 1052 and extends at least partially laterally (e.g., to the side) therefrom. The cut guide 1076 may be positioned relative to other components of the guide 1050 such that, when the guide 1050 is in a desired position on and engaged with the medial cuneiform 202, the cut guide 1076 is positioned over a distal-most portion of the medial cuneiform 202 adjacent to the first tarsometatarsal joint 204.

The cut guide 1076 is shown to include a notch 1078 positioned adjacent to a slot 1080, as shown in at least FIG. 58. The slot 1080 is configured to receive at least a portion of a cutting instrument (e.g., a saw, which may be coupled with a power source or powered driver) at least partially therein/therethrough so as to facilitate guided resection of the medial cuneiform 202 positioned beneath the slot 1080. The slot 1080 is shown to extend from a point adjacent the medial side/edge to a point adjacent the lateral side/edge of the cut guide 1076 and from a top surface through to the bottom surface of the cut guide 1076. Accordingly, the slot 1080 is configured to provide fluid communication between top and bottom surfaces of the cut guide 1076 (as the slot 1080 extends vertically therethrough) but does not provide fluid communication between medial and lateral surfaces of the cut guide 1076. As shown, the slot 1080 is substantially an elongated, rectangular geometry. In some aspects, the slot 1080 may include one or more partial geometries along the length thereof. For example, as shown in at least FIG. 58, the slot 1080 includes circular openings overlapping with the slot 1080 and extending through the cut guide 1076 from the top surface of the cut guide through to the bottom surface thereof.

The notch 1078 includes portions at opposing ends of the slot 1080 (e.g., opposite the longest dimension of the slot 1080), with an interruption in the notch 1078 to accommodate the slot 1080 and the opening thereof. The notch 1078 is shown to be substantially linear and has a depth that is a fraction of that of the slot 1080. The notch 1078 is configured such that a saw blade (or other similar thin, linear component) may be placed in a standing, lengthwise orientation at least partially within the notch 1078 such that the notch 1078 retains the saw blade in the standing, lengthwise position. In some aspects, a physician may position a sawblade within the notch 1078 while the guide 1050 is in a desired position on and coupled with the medial cuneiform 202. Accordingly, in a procedure, a physician may then use fluoroscopic or other radiographic imaging techniques under which the saw blade within the notch 1078 will approximate a cut plane relative to the medial cuneiform 202 along which the slot 1080 of the cut guide 1076 will guide a cut to the medial cuneiform 202. Such a step may not be necessary or required in procedures, but may be performed by a physician in order to visualize the aforementioned cut plane in the medial cuneiform 202 and/or to confirm placement of the guide 1050 relative to the medial cuneiform 202 and the cut plane thereof.

The slot 1080 of the cut guide 1076 is shown to include one or more projections positioned on lateral sides adjacent to the slot 1080 and/or within the slot 1080. In some aspects, the projections may be configured to minimize movement of a saw blade when positioned within/through the slot 1080 when making cuts to bone (e.g., to minimize and/or dampen any vibration of the blade as a result of contact with the medial cuneiform 202).

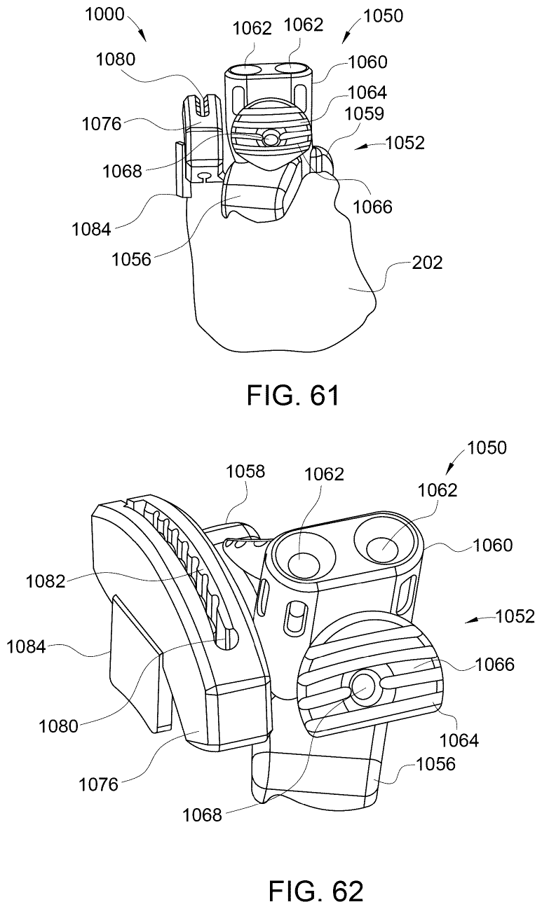

The cut guide 1076 is further shown to include a projection 1084 extending downward from a distal side of the cut guide 1076 (where the distal side of the cut guide 1076 is the side positioned most distally relative to the anatomy of the patient as shown in at least FIG. 59. As shown, the projection 1084 has a lesser lateral dimension (e.g., in the medial-lateral direction) than the cut guide 1076, and extends downward below the bottom surface of the cut guide 1076. The projection 1084, as shown in at least FIGS. 60-61, is configured to extend into the first tarsometatarsal joint 204 adjacent a distal-most surface of the medial cuneiform 202 when placed in a desired position relative to the medial cuneiform 202. In some aspects, the projection 1084 may also be positioned adjacent to or abutting the medial cuneiform 202. Further, it should be understood that the vertical plane defined by the cut slot 1080 is positioned between the projection 1084 and the body 1052 of the guide 1050.

Referring now to FIGS. 70-83, a correction guide 1110 (referred to hereinafter as “guide 1110) (which is a component of the system 1000 along with the guides 1010, 1050) is shown, according to an exemplary embodiment. The guide 1110 may be a single component having various sub-components that are integral with one another, or, as shown in FIGS. 70-83, may include various sub-components that are releasably couplable (e.g., slidably couplable, etc.) with one another.

The guide 1110 is shown to include a body 1112, which is configured to be releasably coupled/is releasably couplable with an insert 1118 and a wire guide 1132, as shown in at least FIG. 74. In some aspects, the guide 1110 may be provided to a physician in a coupled configuration, for example with the insert 1118 received at least partially within a portion of the body 1112 and/or at least a portion of the body received within a portion of the wire guide 1132. In some aspects, the guide 1110 and the aforementioned components thereof may be provided to a physician in a decoupled configuration with such components being coupled with one another while a procedure is performed (e.g., according to a surgical technique or methodology).

The body 1112 is shown to include a first end 1114 positioned opposite the body 1112 from an extension 1130, where at least a portion of the first end 1114 and the extension 1130 define terminal ends of the body 1112. The first end 1114 is shown to have a substantially oval cross-sectional geometry and defines an oval-shaped cavity positioned therein extending from and providing fluid communication between a top surface and a bottom surface of the first end 1114. A pair of legs 1115 are shown to extend downward from a bottom surface of the first end, with each of the legs positioned opposite the oval-shaped cavity 1116 from one another.

The body 1112 is further shown to include a shaft 1124, which may be the same as and/or similar in size and geometry to the shafts described with reference to the guides 1010 and 1050. As shown in at least FIG. 74, the shaft 1124 is integral with the first end 1114 via a connection formed by a rigid linear member extending therebetween. The shaft 1124 is shown to have a substantially oval-shaped cross-sectional geometry, and includes a pair of openings 1126 positioned therein. The openings 1126 are shown to extend from a top surface of the shaft 1124 through to a bottom surface of the shaft 1124. Further, the openings 1126 (which are circular/cylindrical in geometry) are substantially parallel (e.g., central axes thereof are positioned such that the trajectories thereof are substantially parallel). According to some embodiments, the axes of the openings 1126 may also form an oblique axes with one another (e.g., be in a non-parallel configuration). In some aspects, one or both of the openings 1126 may be configured to receive a k-wire or other similar fixation component therethrough, for example to releasably couple the body 1112 (and when in a coupled configuration, the guide 1110) with the medial cuneiform 202, the first metatarsal 206, or both. The shaft 1124 is further shown to include a protrusion 1128 extending downward from the bottom surface of the shaft 1124. As shown, at least a portion of the protrusion 1128 has a substantially semi-spherical or hemi-spherical geometry (as opposed to the legs 1115 which have substantially rectangular geometry). However, in some aspects the protrusion 1128 (and/or the legs 1115) may have alternate geometries based on patient anatomy configured to best promote retention of the guide 1050 in a desired position relative to the medial cuneiform 204 of the patient. The extension 1130 is shown to extend substantially laterally (e.g., outward from the side of) from the shaft 1124 from a point opposite the shaft 1124 from the rigid linear member connecting the shaft 1124 and the first end 1114. Further, the extension 1130 extends from the shaft 1124 in a substantially opposite direction from the aforementioned linear rigid member. As shown, the extension 1130 has a substantially rectangular prism-shaped geometry, which corresponds with releasably coupling feature(s) of the wire guide 1132. However, in some aspects the extension 1130 may have alternate geometries (e.g., cylindrical, triangular prism-shaped, etc.).

The insert 1118 is shown to have a similar oval-shaped geometry to that of the cavity 1116 in which the insert 1118 may be at least partially received, although the insert 1118 is shown to have a lesser lateral dimension to accommodate the reception. The insert 1118 includes a pair of openings 1120, which are shown to extend from a top surface of the insert 1118 through to a bottom surface of the insert 1118. Further, the openings 1120 (which are circular/cylindrical in geometry) are substantially parallel (e.g., central axes thereof are positioned such that the trajectories thereof are substantially parallel). The openings 1120 may be positioned variously about the geometry of the insert 1118. For example, the pair of openings 1120 may be biased toward a top, bottom, left, or right portion of the insert 1118 and thus may be similarly biased relative to the cavity 1116 upon placement therein. Further, the insert 1118 is shown to include a “plus” and “minus” orientation indicated by the corresponding markers on lateral sides thereof. In some aspects, the “plus” orientation may position the openings 1122 such that placing k-wires therethrough provides additional compression across the fusion site. Conversely, the “minus” orientation may position the openings 1122 so as not to provide the same compression across the fusion site. However, in some embodiments the axes of the openings 1120 may also be configured oblique (e.g., non-parallel) relative to one another. The cavity 1116 may include one or more notches positioned adjacent to a top portion thereof so as to accommodate at least a portion of an instrument and facilitate removal of the insert 1118 from the cavity 1116. In some aspects, one or both of the openings 1120 may be configured to receive a k-wire or other similar component therethrough, for example, to releasably couple the insert 1118 (and when in a coupled configuration with other components, the guide 1110) with the medial cuneiform 202, the first metatarsal 206, or both. The insert 1118 is further shown to include a texture 1122, shown in FIGS. 70-83 as one or more ridges disposed on opposing side surfaces of the insert 1118 to facilitate gripping of the insert 1118 by a physician. In some aspects, alternative textures and patterns may be implemented to form the texture 1122.

The wire guide 1132, which is releasably couplable with the extension 1130 of the body 1112, is shown to include a coupling portion 1134 which includes an opening extending therethrough. The opening of the coupling portion 1134 is configured to receive at least a portion of the extension 1130, so as to couple the wire guide with the body 1112, and accordingly the opening extending therethrough as a complimentary geometry to that of the extension 1130. In some aspects, the wire guide 1132 and the body 1112 may be coupled with the first metatarsal and the medial cuneiform 206, 202 such that the fragments of the aforementioned bones are biased toward one another (e.g., so as to apply compression across the fusion site). The coupling portion 1134 is positioned on the wire guide 1132 (e.g., is integral with) adjacent an engagement feature 1136, which may include one or more features/aspects that are the same as and/or similar to those shown and described previously with reference to the engagement features of the guides 1010 and 1050. The engagement feature 1136 is shown to include a substantially tapered geometry, with its greatest lateral dimension adjacent its terminal end. Further, the engagement feature 1136 is configured to include a texture 1138 on a surface thereon to facilitate gripping/retention of the engagement feature 1136 by a physician. Additionally, the engagement feature 1136 is configured to target a downward force in a desired direction so as to prevent skiving of any guide wires placed in order to couple the wire guide 1132 with the first metatarsal 206. In some aspects, the engagement feature 1136 may include a substantially concave outer surface the same as and/or similar to those shown and described previously herein.

As shown, the coupling portion 1134 extends substantially upward from a housing of a bore 1140, where the bore 1140 is configured to guide a trajectory of a of a k-wire or other similar instrument therethrough (e.g., a pin tube or other non-cutting instrumentation), for example to releasably couple the wire guide 1132 to the medial cuneiform 202 and/or the first metatarsal 206. The housing of the bore 1140 is further shown to include an engagement surface 1142 positioned adjacent an end of the bore closest the body 1112 when the wire guide and the body 1112 are in a coupled configuration. The engagement surface 1142 may include a texture or other surface (which may include a substantially rough surface) to prevent movement of the wire guide 1132 while a physician places a k-wire using the trajectory provided by the bore 1140. For example, when such a k-wire is placed along the trajectory of the bore 1140, the engagement surface 1142 may contact and/or engage with a surface of a bone (e.g., the medial cuneiform 202 and/or the first metatarsal 206).

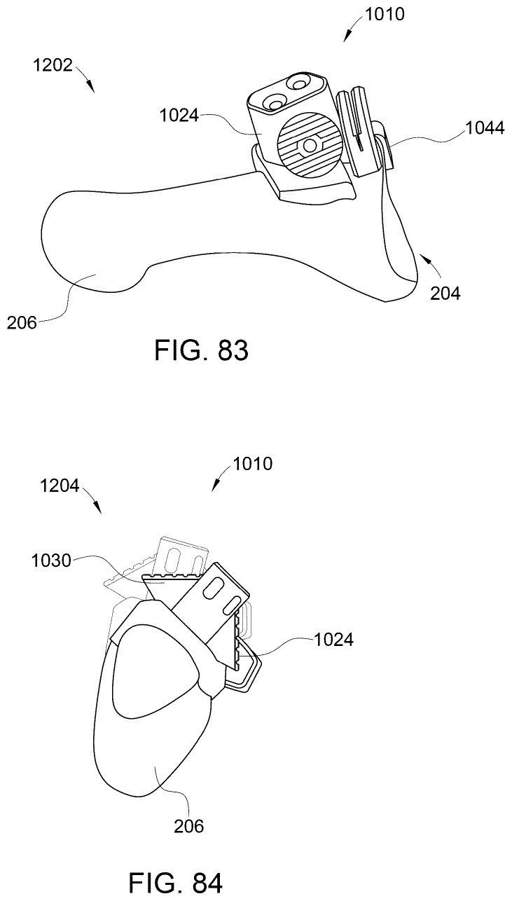

Referring now to FIGS. 83-108, a process or surgical method 1200 is collectively shown, according to an exemplary embodiment. The surgical method 1200 and the various steps thereof may be performed by implementing one or more of the systems and/or components shown and described previously herein, for example the system 1000 including the guides 1010, 1050, and 1110. In some aspects, one or more of the steps in the surgical method 1200 as shown in FIGS. 83-108 may be omitted, repeated, or performed in an alternate order than that shown. Similarly, one or more of the steps in the process 1200 may be performed in an alternate fashion to that shown and described herein. For example, a physician may elect to place a greater or lesser number of k-wires to secure the guide 1010 to the first metatarsal 206 of a patient. Similarly, additional steps (which may also include additional systems/components/instrumentation) may be incorporated into the surgical method 1200. It should also be understood that the surgical method 1200 and steps thereof may include the implementation of common surgical instrumentation including, for example, powered drivers or other powered instruments, medical imaging devices and machinery, traditional surgical instrumentation and other instrumentation common to surgical procedures.

Referring now to FIG. 83, a step 1202 of placing the guide 1010 on the first metatarsal 206 of a patient is shown, according to an exemplary embodiment. In some aspects, the physician may manipulate the guide 1010 using one or more of the engagement features, 1024, 1030 in order to position the guide 1010 on the proximal portion of the first metatarsal 206 as shown in FIG. 83. In some aspects, the projection 1044 may also be positioned within the first tarsometatarsal joint 204 (e.g., between the proximal-most portion of the first metatarsal 206 and the distal-most portion of the medial cuneiform 202. In some aspects, the guide 1010 may be placed on a dorsal surface of the first metatarsal 206 using a superior approach, thus engaging the lower surface 1014 of the guide 1010 with the dorsal surface of the first metatarsal 206.

Referring now to FIG. 84, a step 1204 of positioning the guide 1010 on the first metatarsal 206 of a patient is shown, according to an exemplary embodiment. After the guide 1010 is placed on the first metatarsal 206 in step 1202, the guide 1010 is then manipulated into a desired position in the step 1204. The guide 1010 is a patient-specific guide with one or more features specific to the anatomy of the first metatarsal 206 of the patient including, for example, the lower surface 1014 thereof. Accordingly, in the step 1204 the physician may manipulate the guide 1010 relative to the first metatarsal 206 so as to position the guide 1010 in a desired position relative to the first metatarsal 206. In some aspects, the step 1204 may include a physician manipulating the guide 1010 using the engagement features 1024, 1030 to position patient-specific contours of the guide 1010 (e.g., the lower surface 1014) adjacent the complementary anatomic features of the first metatarsal 206. In some aspects, the guide 1010 (and the patient-specific features thereof) may offer tactile feedback to the physician in order to confirm that the guide 1010 has been positioned appropriately. For example, one or more of the patient-specific features of the guide may be configured to interface with a specific aspect of the first metatarsal 206 of the patient, for example a “flat” portion of the first metatarsal 206 on the lateral side thereof. In the previous example, the guide 1010 may include one or more feature/geometries configured to guide the positioning of the appropriate patient-specific features to the corresponding anatomy of the first metatarsal 206 and provide a “locking in place” tactile feedback to the physician.

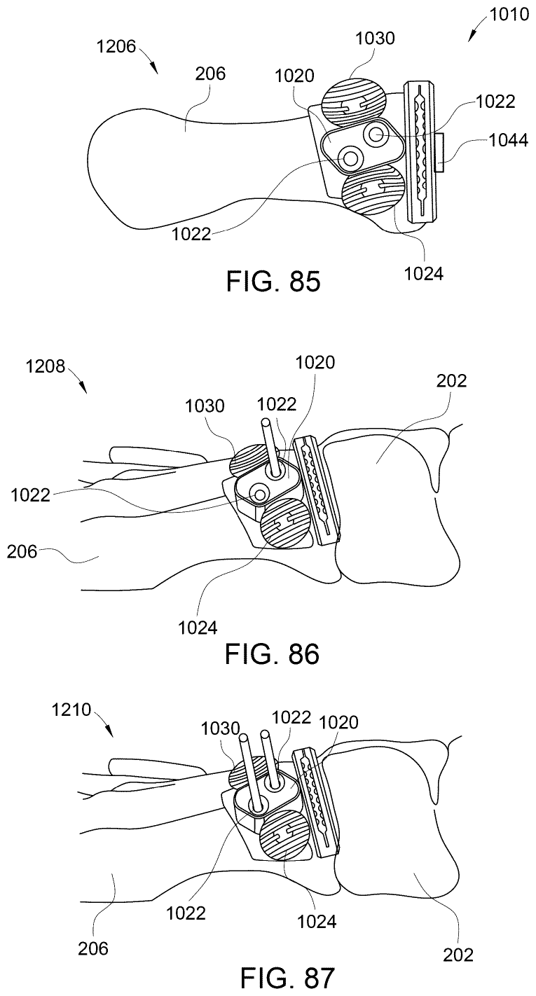

Referring now to FIG. 85, a step 1206 of evaluating the fit and/or position of the guide 1010 on the first metatarsal 206 of a patient is shown, according to an exemplary embodiment. The step 1206 may include the physician evaluating various aspects of the fit of the guide 1010 relative to the first metatarsal 206, for example the position of the cut guide 1036 relative to the proximal-most portion of the first metatarsal 206. Additionally, the physician may manipulate the guide 1010 using the engagement features 1024, 1030 in a side to side or twisting manner to ensure the guide 1010 is properly seated in the desired position (which may be indicated by the “locking” feedback provided by one or more components of the guide 1010 as described with reference to the step 1204).

Referring now to FIG. 86, a step 1208 of coupling the guide 1010 with the first metatarsal 206 of a patient with a first k-wire is shown, according to an exemplary embodiment. The step 1208 is shown to include the physician, using a powered instrument, placing a k-wire into and through one of openings 1022 of the shaft 1020 of the guide 1010 and into the first metatarsal 206 of the patient. In some aspects, a physician may place a k-wire in the proximal-most opening 1022 in the step 1208, or may place a second k-wire in the distal-most opening 1022. In some aspects, the physician may grip the engagement features 1024, 1030 while placing the aforementioned k-wire through the opening 1022.

Referring now to FIG. 87, a step 1210 of coupling the guide 1010 with the first metatarsal 206 of a patient using a second k-wire is shown, according to an exemplary embodiment. Similar to the step 1208, the step 1210 includes the physician, using a powered instrument, placing a k-wire into and through one of openings 1022 of the shaft 1020 of the guide 1010 and into the first metatarsal 206 of the patient. In the step 1210, the k-wire is placed in the opening 1022 of the guide 1010 that is vacant (e.g., the opening 1022 that does not have a k-wire placed therein from the physician performing the step 1208). Similar to the step 1208, the physician may grip the engagement features 1024, 1030 while placing the second k-wire through the vacant opening 1022.

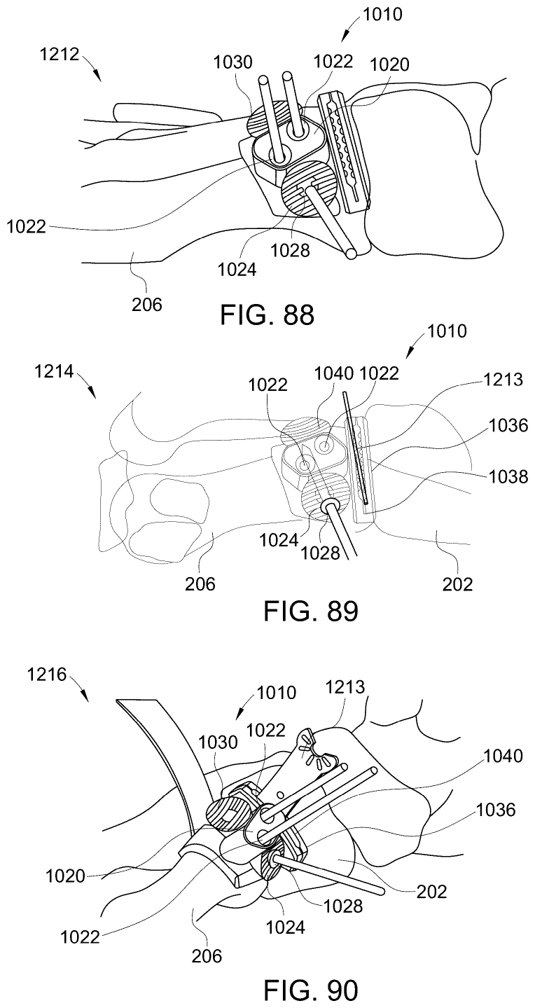

Referring now to FIG. 88, a step 1212 of securing the guide 1010 to the first metatarsal 206 of a patient is shown, according to an exemplary embodiment. Once the guide 1010 has been coupled with the first metatarsal 206 as shown and described with reference to the steps 1208, 1210, the guide 1010 is then secured to the first metatarsal 206 by placing at least one olive wire through at least one of the openings 1028, 1034 of the engagement features 1024, 1030 such that the rounded, spherical portion of the olive wire (e.g., the “olive”) is positioned adjacent to or abutting the engagement feature 1024 and/or 1030. The olive wires of the step 1212 provide supplemental securing/coupling of the guide 1010 with the first metatarsal 206, as the openings 1022 may be substantially parallel to one another and, accordingly, the guide 1010 may be decoupled from the first metatarsal 206 over the k-wires inserted in the openings 1022 without supplement coupling/securing of the guide 1010.

Referring now to FIG. 89, a step 1214 of confirming positioning of the guide 1010 on the first metatarsal 206 of a patient is shown, according to an exemplary embodiment. In some aspects, the step 1214 may include a physician implementing fluoroscopic or other imaging modalities intraoperatively. For example, in the step 1214 the physician may position a saw blade 1213 (shown in FIG. 89) vertically within the notch 1038 of the cut guide 1036 such that the blade 1213 stands upright within the notch 1038. The physician may then examine the placement of the guide 1010 as well as the blade 1213 using one or more imaging modalities. The guide 1010 may be a radiolucent material which is transparent under fluoroscopy, whereas the blade 1213 may be a radiopaque material which is visible under fluoroscopy. Accordingly, the physician may use the blade 1213 on the fluoroscopy image to visualize the cut plane guided by the slot 1040 of the cut guide 1040 and confirm, based on the cut plane and resultant proximal section of the first metatarsal 206 to be resected, that the guide 1010 is positioned as desired. If the physician were to determine that the guide 1010 was not positioned as desired, one or more of the steps 1202, 1204, 1206, 1208, 1210, and/or 1212 may be repeated until the guide 1010 is confirmed in the step 1214 to be in the desired position.

Referring now to FIG. 90, a step 1216 of resecting the first metatarsal 206 using the guide 1010 is shown, according to an exemplary embodiment. The step 1216 may include a physician implementing a powered instrument, for example a reciprocating or sagittal saw (which may be coupled with the blade 1213 used for visualization in the step 1214, or may be coupled with a different blade), to perform a resection cut to the proximal portion of the first metatarsal 206 by inserting the blade of the powered instrument within the slot 1040 of the guide 1010. Once the blade of the powered instrument is inserted within the slot 1040, power may be applied to the device in order to actuate the saw blade and, in conjunction with a force applied by the physician, resect a proximal portion of the first metatarsal 206. In some aspects, the physician may insert a guard instrument (e.g., as shown in FIG. 90) to protect a second metatarsal of the patient and/or any adjacent tissue from being contacted by the blade of the saw while performing the resection cut.

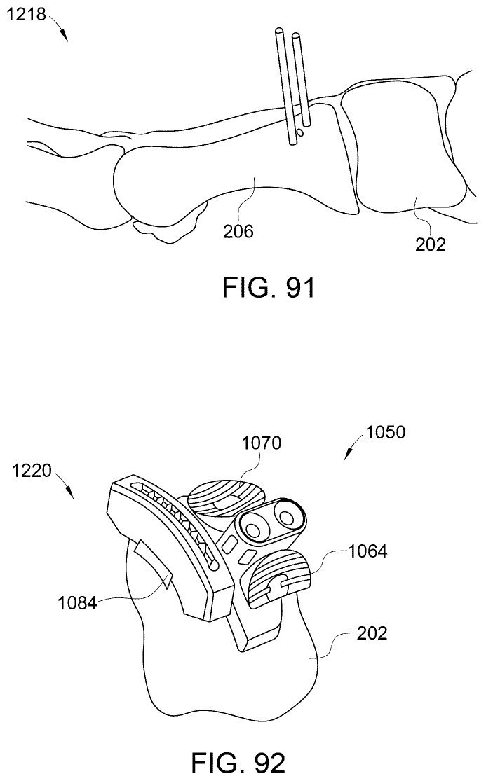

Referring now to FIG. 91, a step 1218 of decoupling the guide 1010 from the first metatarsal 206 of a patient is shown, according to an exemplary embodiment. In performing the step 1218, the physician may first remove olive wires coupling the guide 1010 with the first metatarsal 206 via the openings 1028, 1034 of the engagement features 1024, 1030. Once any olive wires have been removed, the guide 1010 may be decoupled from the first metatarsal 206 by removing the guide 1010 upward and over the k-wires which were previously inserted in the openings 1022. Further, in the step 1218 the physician may also remove the resected portion of the first metatarsal 206 and/or other resected tissue from the joint space (e.g., the first tarsometatarsal joint 204). It should be understood that in performing the step 1218, the k-wires previously inserted in the first metatarsal 206 through the openings 206 are not removed/decoupled from the first metatarsal.

Referring now to FIG. 92, a step 1220 of placing the guide 1050 on the medial cuneiform 202 of a patient is shown, according to an exemplary embodiment. In some aspects, the physician may manipulate the guide 1050 using one or more of the engagement features, 1064, 1070 in order to position the guide 1050 on the distal portion of the medial cuneiform 202 as shown in FIG. 83. In some aspects, the projection 1084 may also be positioned within the first tarsometatarsal joint 204 (e.g., between the proximal-most portion of the first metatarsal 206 and the distal-most portion of the medial cuneiform 202. In some aspects, the guide 1050 may be placed on a dorsal surface of the medial cuneiform 202 using a superior approach, thus engaging the lower surface 1054 of the guide 1050 with the dorsal surface of the medial cuneiform 202.

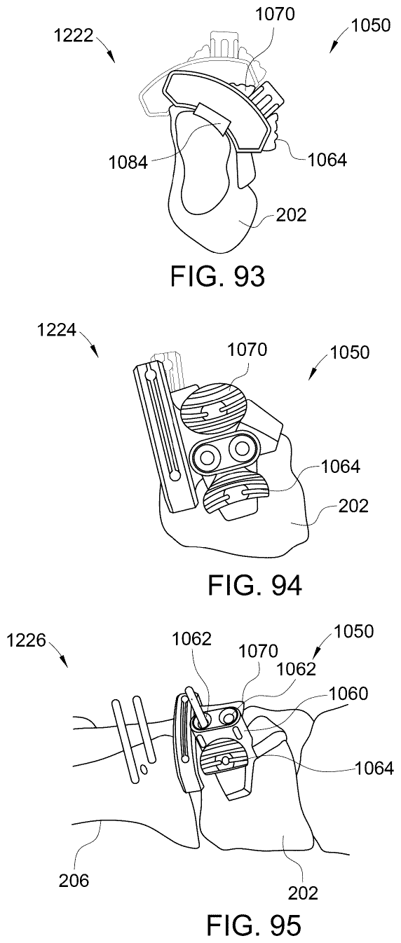

Referring now to FIG. 93, a step 1222 of positioning the guide 1050 on the medial cuneiform 202 of a patient is shown, according to an exemplary embodiment. After the guide 1050 is placed on the medial cuneiform 202 in step 1220, the guide 1050 is then manipulated into a desired position in the step 1222. The guide 1050 is a patient-specific guide with one or more features specific to the anatomy of the medial cuneiform 202 of the patient including, for example, the lower surface 1054 thereof. Accordingly, in the step 1222 the physician may manipulate the guide 1050 relative to the medial cuneiform 202 so as to position the guide 1050 in a desired position relative to the medial cuneiform 202. In some aspects, the step 1204 may include a physician manipulating the guide 1050 using the engagement features 1064, 1070 to position patient-specific contours of the guide 1050 (e.g., the lower surface 1054) adjacent the complementary anatomic features of the medial cuneiform 202. In some aspects, the guide 1050 (and the patient-specific features thereof) may offer tactile feedback to the physician in order to confirm that the guide 1050 has been positioned appropriately. For example, one or more of the patient-specific features of the guide may be configured to interface with a specific aspect of the medial cuneiform 202 of the patient, for example a “flat” portion of the medial cuneiform 202 on the lateral side thereof. In the previous example, the guide 1050 may include one or more feature/geometries configured to guide the positioning of the appropriate patient-specific features to the corresponding anatomy of the medial cuneiform 202 and provide a “locking in place” tactile feedback to the physician.

Referring now to FIG. 94, a step 1224 of evaluating the fit and/or position of the guide 1050 on the medial cuneiform 202 of a patient is shown, according to an exemplary embodiment. The step 1224 may include the physician evaluating various aspects of the fit of the guide 1050 relative to the medial cuneiform 202, for example the position of the cut guide 1076 relative to the distal-most portion of the medial cuneiform 202. Additionally, the physician may manipulate the guide 1050 using the engagement features 1064, 1070 in a side to side or twisting manner to ensure the guide 1050 is properly seated in the desired position (which may be indicated by the “locking” feedback provided by one or more components of the guide 1050 as described with reference to the step 1222).

Referring now to FIG. 95, a step 1226 of coupling the guide 1050 with the medial cuneiform 202 of a patient with a first k-wire is shown, according to an exemplary embodiment. The step 1226 is shown to include the physician, using a powered instrument, placing a k-wire into and through one of openings 1062 of the shaft 1060 of the guide 1050 and into the medial cuneiform 202 of the patient. In some aspects, the physician may place a k-wire in the proximal-most opening 1062 in the step 1226, or may place a second k-wire in the distal-most opening 1062. In some aspects, the physician may grip the engagement features 1064, 1070 while placing the aforementioned k-wire through the opening 1062.

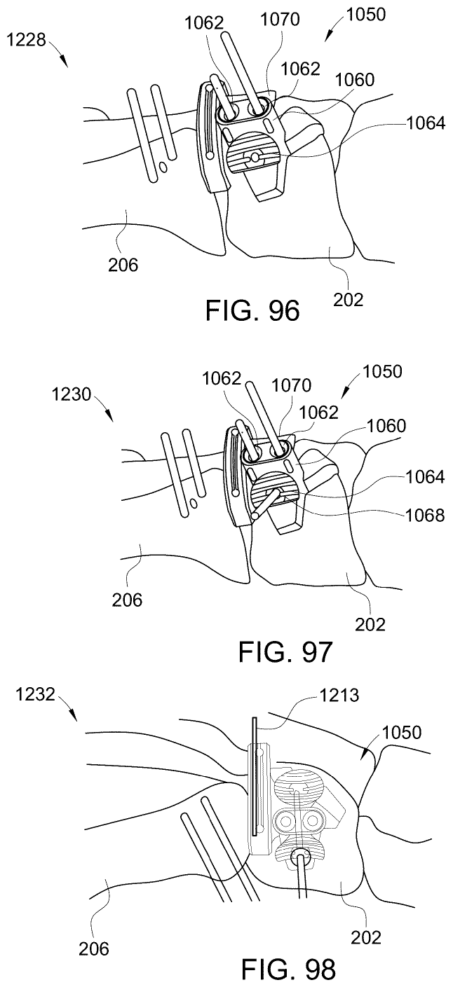

Referring now to FIG. 96, a step 1228 of coupling the guide 1050 with the medial cuneiform 202 of a patient using a second k-wire is shown, according to an exemplary embodiment. Similar to the step 1226, the step 1228 includes the physician, using a powered instrument, placing a k-wire into and through one of openings 1062 of the shaft 1060 of the guide 1050 and into the medial cuneiform 202 of the patient. In the step 1228, the k-wire is placed in the opening 1062 of the guide 1050 that is vacant (e.g., the opening 1062 that does not have a k-wire placed therein from the physician performing the step 1208). Similar to the step 1226, the physician may grip the engagement features 1064, 1070 while placing the second k-wire through the vacant opening 1062. It should be understood that, in some aspects, the k-wires inserted in the medial cuneiform 202 may be shorter in length (and thus not extend as high from the surface of the medial cuneiform 202) as those inserted in the first metatarsal 206. Further, the k-wires inserted in the first metatarsal 206 and the medial cuneiform 202 may be staggered in length relative to the opposite k-wire inserted in the same bone.

Referring now to FIG. 97, a step 1230 of securing the guide 1050 to the medial cuneiform 202 of the patient is shown, according to an exemplary embodiment. Once the guide 1050 has been coupled with the medial cuneiform 202 as shown and described with reference to the steps 1226, 1228, the guide 1050 is then secured to the medial cuneiform 202 by placing at least one olive wire through at least one of the openings 1068, 1074 of the engagement features 1064, 1070 such that the rounded, spherical portion of the olive wire (e.g., the “olive”) is positioned adjacent to or abutting the engagement feature 1064 and/or 1070. The olive wires of the step 1230 provide supplemental securing/coupling of the guide 1050 with the medial cuneiform 202, as the openings 1062 may be substantially parallel to one another and, accordingly, the guide 1050 may be decoupled from the medial cuneiform 202 over the k-wires inserted in the openings 1062 without supplement coupling/securing of the guide 1050.

Referring now to FIG. 98, a step 1232 of confirming positioning of the guide 1050 on the medial cuneiform 202 of the patient is shown, according to an exemplary embodiment. In some aspects, the step 1232 may include the physician implementing fluoroscopic or other imaging modalities intraoperatively. For example, in the step 1232 the physician may position a saw blade 1213 (shown in FIG. 98) vertically within the notch 1078 of the cut guide 1076 such that the blade 1213 stands upright within the notch 1078. The physician may then examine the placement of the guide 1050 as well as the blade 1213 using one or more imaging modalities. The guide 1050 may be a radiolucent material which is transparent under fluoroscopy, whereas the blade 1213 may be a radiopaque material which is visible under fluoroscopy. Accordingly, the physician may use the blade 1213 on the fluoroscopy image to visualize the cut plane guided by the slot 1080 of the cut guide 1080 and confirm, based on the cut plane and resultant distal section of the medial cuneiform 202 to be resected, that the guide 1050 is positioned as desired. If the physician were to determine that the guide 1050 was not positioned as desired, one or more of the steps 1220, 1222, 1224, 1226, 1228, and/or 1230 may be repeated until the guide 1050 is confirmed in the step 1232 to be in the desired position.

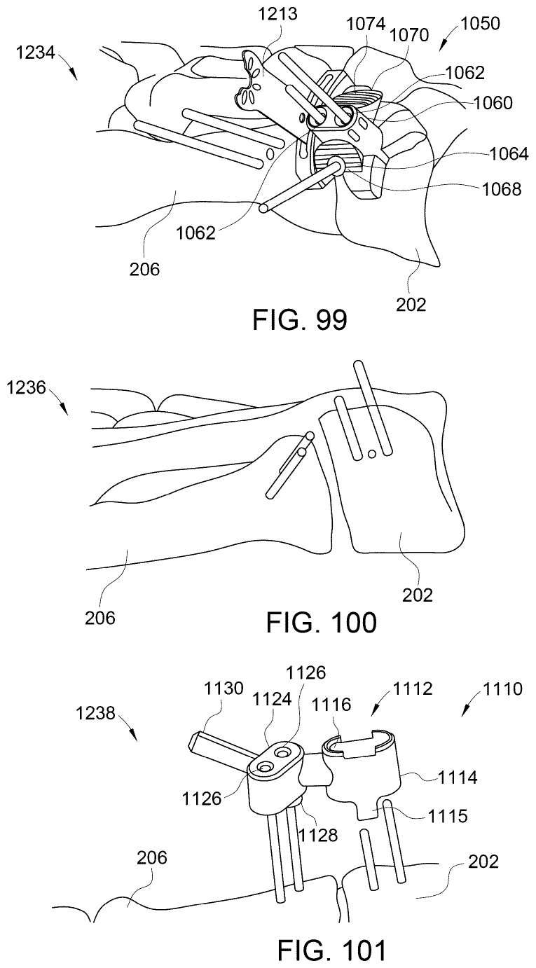

Referring now to FIG. 99, a step 1234 of resecting the medial cuneiform 202 using the guide 1050 is shown, according to an exemplary embodiment. The step 1234 may include the physician implementing a powered instrument, for example a reciprocating or sagittal saw (which may be coupled with the blade 1213 used for visualization in the step 1232, or may be coupled with a different blade), to perform a resection cut to the distal portion of the medial cuneiform 202 by inserting the blade of the powered instrument within the slot 1080 of the guide 1050. Once the blade of the powered instrument is inserted within the slot 1080, power may be applied to the device in order to actuate the saw blade and, in conjunction with a force applied by the physician, resect a distal portion of the medial cuneiform 202. In some aspects, the physician may insert a guard instrument (e.g., the same as or similar to that shown in FIG. 90) to protect anatomy medial of the medial cuneiform 202 of the patient and/or any adjacent tissue from being contacted by the blade of the saw while performing the resection cut.

Referring now to FIG. 100, a step 1236 of decoupling the guide 1050 from the medial cuneiform 202 of the patient is shown, according to an exemplary embodiment. In performing the step 1236, the physician may first remove olive wires coupling the guide 1050 with the medial cuneiform 202 via the openings 1068, 1074 of the engagement features 1064, 1070. Once any olive wires have been removed, the guide 1050 may be decoupled from the medial cuneiform 202 by removing the guide 1050 upward and over the k-wires which were previously inserted in the openings 1062. Further, in the step 1236 the physician may also remove the resected portion of the medial cuneiform 202 and/or other resected tissue from the joint space (e.g., the first tarsometatarsal joint 204). It should be understood that in performing the step 1236, the k-wires previously inserted in the medial cuneiform 202 through the openings 206 are not removed/decoupled from the first metatarsal (but rather provide a reference location for the subsequent step).

Referring now to FIG. 101, a step 1238 of coupling the guide 1110 with the k-wires inserted in the first metatarsal 206 is shown, according to an exemplary embodiment. It should be understood that in performing the step 1238 and the following steps involving the guide 1110, the guide is implemented in a decoupled/disassembled configuration. As shown in FIG. 101, the k-wires inserted in the first metatarsal 206 extend further (e.g., higher) from the surface of the bone than those inserted in the medial cuneiform 202. Accordingly, the k-wires of the first metatarsal 206 may be aligned with and inserted into and through the openings 1126 of the shaft 1124 of the guide 1110 without any interference from the k-wires of the medial cuneiform 202. For example, in aligning the k-wires of the first metatarsal 206 with the openings 1126, the body 1112, first portion 1114, and extension 1130 of the guide 1110 may be positioned substantially above the k-wires of the medial cuneiform 202. Once the k-wires of the first metatarsal 206 are positioned at least partially within the openings 1126, the guide 1110 is slid along a portion of the length of the aforementioned k-wires such that the first portion 1114 is positioned above and/or adjacent to the medial cuneiform 202.

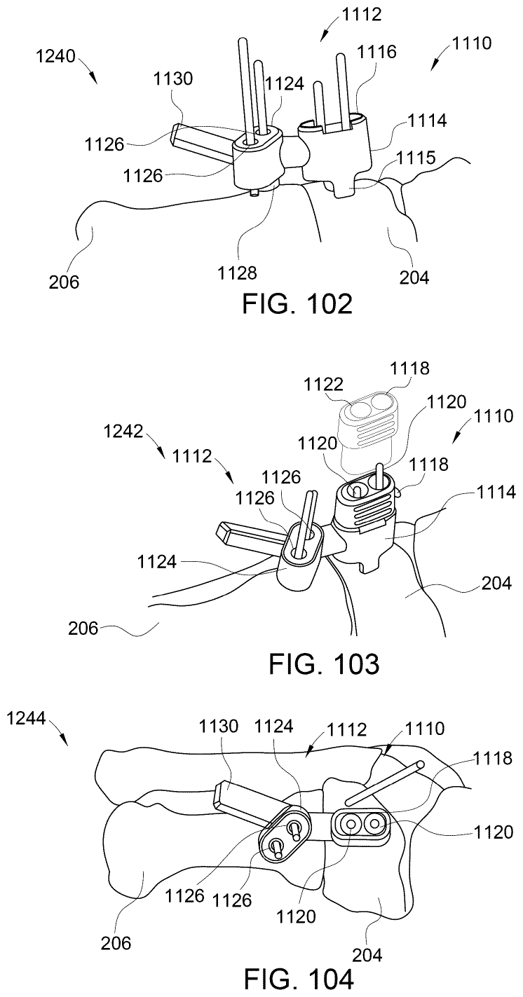

Referring now to FIG. 102, a step 1240 of coupling the guide 1110 with the k-wires of the medial cuneiform 202 is shown, according to an exemplary embodiment. The guide 1110 is positioned above and/or adjacent to the k-wires of the first metatarsal 206 at the completion of the step 1238, and in the step 1240 the first portion 1114 and the cavity 1116 are aligned with the k-wires of the medial cuneiform 202. In some aspects, the k-wires of the first metatarsal 206 and the medial cuneiform 202 may not be co-planar or aligned with the opposing pair (e.g., relative to the first ray of the foot of the patient, the k-wires of the first metatarsal 206 may be biased medially relative to the k-wires of the medial cuneiform 202). Accordingly, in aligning the cavity 1116 above the k-wires of the medial cuneiform 202 the physician may manipulate the first metatarsal 206 in one or more directions (e.g., rotate in a clockwise direction when viewed from a posterior-to-anterior perspective, bias the first metatarsal 206 laterally to close the intermetatarsal angle between the first metatarsal 206 and the second metatarsal, and/or reposition the first metatarsal 206 in the sagittal plane so as to raise or lower a distal portion of the first metatarsal 206 relative to the proximal portion thereof). In performing one or more of the aforementioned manipulations, the cavity 1116 may be positioned above the k-wires of the medial cuneiform 202, and the guide 1110 may be slid further down the k-wires of the first metatarsal 206 such that the k-wires of the medial cuneiform 202 are received within the cavity 1116 and extend therethrough. The step 1240 is complete with the guide 1110 abuts or is positioned just above the surfaces of the first metatarsal 206 and the medial cuneiform 202 such that the pair of legs 1115 and the protrusion 1128 contact the appropriate bony surfaces (medial cuneiform 202 and first metatarsal 206, respectively), with the k-wires of the first metatarsal 206 extending through the openings 1126 and the k-wires of the medial cuneiform 202 extending through the cavity 1116 of the guide 1110.

Referring now to FIG. 103, a step 1242 of coupling the insert 1118 with the guide 1110 and the k-wires of the medial cuneiform 202 is shown, according to an exemplary embodiment. Once the guide 1110 is placed in the step 1240, the k-wires of the medial cuneiform 202 are retained within the cavity 1116. Accordingly, the insert 1118 may be positioned above the upper ends of the k-wires of the medial cuneiform 202 such that the openings 1120 thereof are aligned with the k-wires. The insert 1118 may then be moved downward such that the k-wires of the medial cuneiform 202 are received into and through the openings 1120, and the insert 1118 is translated along the length of the k-wires of the medial cuneiform 202 until the insert 1118 is received at least partially therein the cavity 1116 of the guide 1110. In performing the step 1242, the physician may grasp the texture 1122 of the insert 1118 in order to facilitate gripping thereof. In some aspects, the insert 1118 may include one or more geometric features positioned on an outer surface thereof that are configured to facilitate releasable coupling and/or engagement with the first portion 1114 that defines the cavity 1116. Once the insert 1118 has been appropriately seated in/adjacent to the cavity 1116, the first metatarsal 206 (and in some aspects, the medial cuneiform 204) have been manipulated to a corrected position and the resected surfaces of the first metatarsal 206 and the medial cuneiform 202 abut one another. As mentioned previously, the openings 1120 of the insert 1118 may not be centrally positioned relative to the insert 1118. Accordingly, the step 1242 includes the determination, by the physician, of the proper orientation of the insert 1118 relative to the cavity 1116 prior to insertion therein. For example, in some aspects a physician may elect to use the “plus” orientation of the insert 1118, thus providing additional compression across the fusion site, or may elect to use the “minus” orientation of the insert 1118 (which does not provide the same compression across the joint site).

Referring now to FIG. 104, a step 1244 of placing primary temporary fixation across the first tarsometatarsal joint 204 is shown, according to an exemplary embodiment. As shown in FIG. 104, the step 1244 includes placing a k-wire or other similar component across the first tarsometatarsal joint 204 from the proximal side of the first tarsometatarsal joint 204 (in the medial cuneiform 202) to the distal side of the joint (in the first metatarsal 206). As shown, the k-wire of the step 1244 is placed in the medial cuneiform 202 medially relative to the guide 1110, although in some aspects the k-wire may be positioned alternatively. In some aspects, the placement of the temporary fixation may be guided, for example by one or more openings of the wire guide 1132 and/or the body 1112.

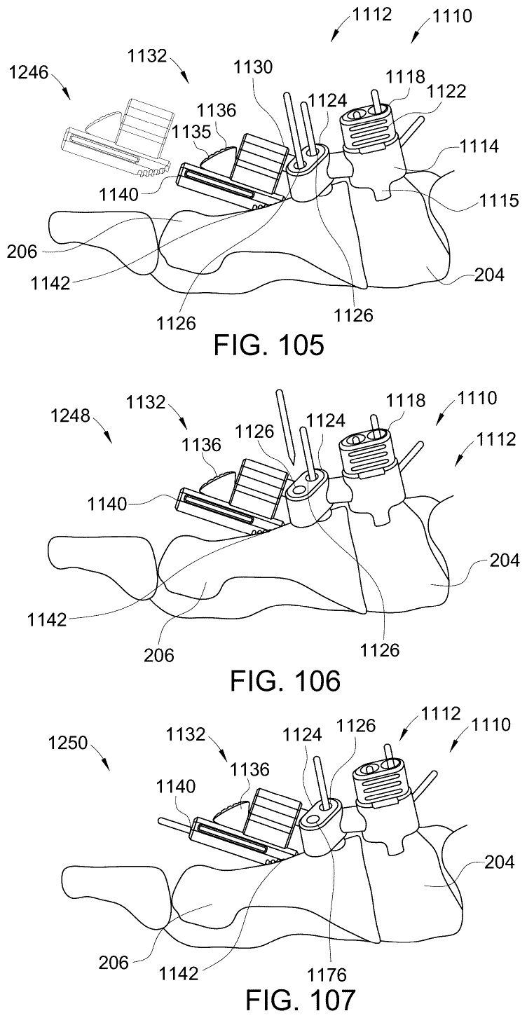

Referring now to FIG. 105, a step 1246 of coupling the second portion 1132 of the guide 1110 with the extension 1130 of the body 1112 of the guide 1110 is shown, according to an exemplary embodiment. As shown in FIG. 105, the second portion 1132 is positioned such that the opening 1134 thereof is positioned adjacent to the terminal end of the extension 1130. Accordingly, the second portion 1132 is then translated toward the extension 1130 such that the extension 1130 is at least partially received within the opening 1134.

Referring now to FIG. 106, a step 1248 of removing the distal k-wire placed in the first metatarsal 206 is shown, according to an exemplary embodiment. The distal-most k-wire placed within the distal-most of the openings 1126 is removed, using a powered instrument (e.g., similar to that with which it was placed), so as to prevent interference with any subsequently placed k-wires.

Referring now to FIG. 107, a step 1250 of coupling the wire guide 1132 with the first metatarsal 206 via a k-wire is shown, according to an exemplary embodiment. In the step 1250, a k-wire is placed within the opening 1240 so as to provide temporary fixation of the first metatarsal 206 and the medial cuneiform 202 across the fusion site. In some aspects, the trajectory/path of the k-wire may intersect with that of the previously removed distal-most k-wire of the openings 126. In some aspects, a secondary temporary fixation k-wire may be placed, either freehand or guided by an additional feature of the system 1100, so as to provide additional stability to the temporary fixation of the fusion site.

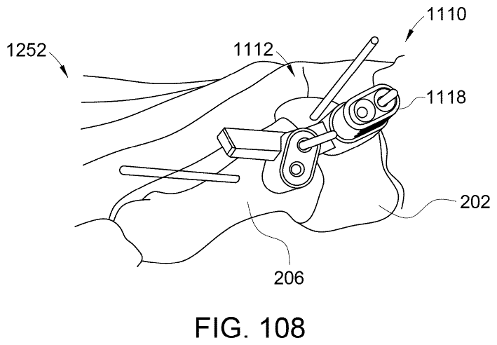

Referring now to FIG. 108, a step 1252 of removing the wire guide 1132 from the first metatarsal 206 is shown, according to an exemplary embodiment. The trajectory of the k-wire placed within the opening 1140 in the step 1250 is parallel to the axis of the opening 1040 and, thus, the wire guide 1132 can be slid along the k-wire and decoupled from both the first metatarsal 206 and the body 1112. In some aspects, the step 1252 may also include removal of the body 1112 from the medial cuneiform 202. Following the step 1252, preparation may be performed for one or more hardware fixation modalities (e.g., screw constructs, plating, intramedullary nails, etc.) with the one or more modalities then applied across the fusion site.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A surgical system, comprising:
a first guide component configured to releasably couple with a first bone of a patient;
a second guide component configured to releasably couple with a second bone of the patient;
a cut guide; and
a correction guide.

2. The surgical system of claim 1, wherein the first guide component comprises one or more features configured to the anatomy of the patient.

3. The surgical system of claim 1, wherein the first guide component comprises at least one bone-interfacing surface.

4. The surgical system of claim 3, wherein the at least one bone-interfacing surface comprises at least one curvature that is complementary to at least one curvature of the first bone of the patient.

5. The surgical system of claim 1, wherein the first guide component further comprises a wire guide, wherein the wire guide comprises a pair of openings extending therethrough from a top surface to a bottom surface and establishing fluid communication therebetween.

6. The surgical system of claim 5, wherein the wire guide is integral with the first guide component.

7. The surgical system of claim 1, wherein the first guide component comprises a top surface positioned opposite a bottom surface, and a medial portion opposite from a lateral portion, wherein the top and bottom surfaces extend at least partially from the medial side to the lateral side of the body.

8. The surgical system of claim 7, wherein at least one of the medial portion and the lateral portion comprises an engagement feature extending upward from the top surface.

9. The surgical system of claim 1, wherein the first guide component comprises one or more features configured to the anatomy of the patient.

10. The surgical system of claim 1, wherein the second guide component comprises at least one bone-interfacing surface.

11. The surgical system of claim 10, wherein the at least one bone-interfacing surface comprises at least one curvature that is complementary to at least one curvature of the second bone of the patient.

12. The surgical system of claim 1, wherein the second guide component further comprises a wire guide, wherein the wire guide comprises a pair of openings extending therethrough from a top surface to a bottom surface and establishing fluid communication therebetween.

13. The surgical system of claim 12, wherein the wire guide is integral with the first guide component.

14. The surgical system of claim 1, wherein the second guide component comprises a top surface positioned opposite a body from a bottom surface, and a medial portion opposite the body from a lateral portion, wherein the top and bottom surfaces extend at least partially from the medial side to the lateral side of the body.

15. The surgical system of claim 14, wherein at least one of the medial portion and the lateral portion comprises an engagement feature extending upward from the top surface.

16. The surgical system of claim 1, further comprising at least one first stabilization wire configured to couple the second guide component with the first bone of the patient and at least one second stabilization wire configured to releasably couple the second guide component with the second bone of the patient.

17. The surgical system of claim 16, wherein the cut guide is configured to engage with the first bone of the patient via the at least one first stabilization wire.

18. The surgical system of claim 17, wherein the cut guide is configured to engage with the second bone of the patient via the at least one second stabilization wire.

19. A surgical method, comprising:
coupling a first guide with a first bone of a patient using at least one first stabilization wire;
removing the first guide from the first bone;
coupling a second guide with a second bone of the patient using at least one second stabilization wire;
removing the second guide from the second bone;
engaging a cut guide with the first bone of the patient via the at least one first stabilization wire;
disengaging the cut guide from the first bone of the patient;
engaging the cut guide with the second bone of the patient via the at least one second stabilization wire; and
disengaging the cut guide from the second bone of the patient.

20. The surgical method of claim 19, further comprising:
performing at least one resectioning cut to the first bone, wherein the at least one resectioning cut is guided by a slot disposed on the cut guide; and
performing at least one resectioning cut to the second bone, wherein the at least one resectioning cut is guided by the slot disposed on the cut guide.

21. A surgical system, comprising:
a first guide;
a second guide; and
a correction guide.

22. The surgical system of claim 21, wherein the first guide is configured to guide a blade to cut a proximal end of a first metatarsal of a patient.

23. The surgical system of claim 22, wherein the first guide is a patient-specific guide.

24. The surgical system of claim 23, wherein the second guide is configured to guide a blade to cut a distal end of a medial cuneiform of the patient.

25. The surgical system of claim 24, wherein the second guide is a patient-specific guide.

26. The surgical system of claim 25, wherein the correction guide is configured to releasably couple with the first metatarsal and the medial cuneiform of the patient.

27. The surgical system of claim 26, wherein the correction guide is modular and comprises a proximal portion and a distal portion that are releasably couplable with one another.

28. The surgical system of claim 22, wherein the first guide comprises:
a cut slot positioned at a proximal portion thereof; and
at least one opening configured to receive a k-wire at least partially therethrough so as to facilitate coupling of the first guide with the first metatarsal.

29. The surgical system of claim 28, wherein the first guide further comprises at least one engagement feature.

30. The surgical system of claim 29, wherein the at least one opening extends at least partially through the at least one engagement feature.

31. The surgical system of claim 30, wherein the at least one engagement feature comprises a medial engagement feature and a lateral engagement feature, and wherein the at least one opening comprises a first opening extending at least partially through the medial engagement feature and a second opening extending at least partially through the lateral engagement feature.

32. The surgical system of claim 29, wherein the second guide comprises:
a cut slot positioned at a distal portion thereof; and
at least one opening configured to receive a k-wire at least partially therethrough so as to facilitate coupling of the first guide with the medial cuneiform.

33. The surgical system of claim 32, wherein the second guide further comprises at least one engagement feature.

34. The surgical system of claim 33, wherein the at least one opening extends at least partially through the at least one engagement feature.

35. The surgical system of claim 34, wherein the at least one engagement feature comprises a medial engagement feature and a lateral engagement feature, and wherein the at least one opening comprises a first opening extending at least partially through the medial engagement feature and a second opening extending at least partially through the lateral engagement feature.

36. A surgical system, comprising:
a first guide, comprising:
a cut slot positioned at a proximal portion thereof;
a first engagement feature;
a second engagement feature;
a first opening extending at least partially through the first engagement feature; and
a second opening extending at least partially through the second engagement feature;
a second guide, comprising:
a cut slot positioned at a distal portion thereof;
a first engagement feature;
a second engagement feature;
a first opening extending at least partially through the first engagement feature; and
a second opening extending at least partially through the second engagement feature; and
a correction guide comprising:
a proximal portion; and
a distal portion, wherein the proximal and distal portions are releasably couplable with one another.

37. The surgical system of claim 36, wherein the first and second guides are patient-specific guides.

38. The surgical system of claim 37, wherein the correction guide is a patient-specific guide.

39. The surgical system of claim 38, wherein the first guide is configured to be releasably couplable with a first metatarsal of a patient, and wherein the second guide is configured to be releasably couplable with a medial cuneiform of the patient.

40. A method of correcting a bunion, comprising:
engaging a first guide with a first metatarsal of a patient;
releasably coupling the first guide with the first metatarsal of the patient using one or more stabilization wires;
performing a resection cut to the proximal portion of the first metatarsal through a cut slot positioned at a proximal end of the first guide;
engaging a second guide with a medial cuneiform of the patient;
releasably coupling the second guide with the medial cuneiform of the patient using one or more stabilization wires;
performing a resection cut to the distal portion of the medial cuneiform through a cut slot positioned at a distal end of the second guide; and
engaging a correction guide with the first metatarsal and medial cuneiform to bias the first metatarsal and medial cuneiform into a corrected position.

41. The surgical system of claim 1, wherein the first bone is a first metatarsal and the second bone is a medial cuneiform.
